(19) United States
(12) Patent Application Publication
(10) Pub. No.: US 2026/0117192 A1
(43) Pub. Date: Apr. 30, 2026

(54) METHODS OF PRODUCING A TUMOR AVATAR

(71) Applicant: ARIEL SCIENTIFIC INNOVATIONS LTD., Ariel (IL)

(72) Inventors: Gabriela ROZIC, Ariel (IL); Igor KOMAN, Ariel (IL)

(21) Appl. No.: 19/433,537

(22) Filed: Dec. 26, 2025

Related U.S. Application Data
(63) Continuation of application No. PCT/IL2024/050631, filed on Jun. 27, 2024.

(30) Foreign Application Priority Data
Jun. 27, 2023 (IL) 304102

(51) Int. Cl. C12N 5/09; C12N 5/00; C12N 5/071; C12N 5/077

(52) U.S. Cl. CPC C12N 5/0693; C12N 5/0062; C12N 5/0656; C12N 5/069; C12N 2501/02; C12N 2501/117; C12N 2501/119; C12N 2501/12; C12N 2501/195; C12N 2501/345; C12N 2501/415; C12N 2503/02; C12N 2503/04; C12N 2513/00; C12N 2533/52

(57) ABSTRACT

Three-dimensional tumor avatars comprising an artificial scaffold, tumor cells, tumor associated endothelial cells and tumor associated fibroblast cells are provided. Kits comprising the 3D tumor avatars, methods of producing the 3D tumor avatars and methods of using the 3D tumor avatars are also provided.

### CROSS REFERENCE TO RELATED APPLICATIONS

This application is a Bypass Continuation of PCT Patent Application No. PCT/IL2024/050631 having International filing date of Jun. 27, 2024, which claims the benefit of priority of Israeli Patent Application No. 304102, filed Jun. 27, 2023, the contents of which are all incorporated herein by reference in their entirety.

### FIELD OF INVENTION

The present invention is in the field of cancer diagnostics.

### BACKGROUND OF THE INVENTION

The tumor microenvironment (TME) constitutes a permissive milieu for the induction, selection, and expansion of cancer cells and has been shown to play diverse and critical roles in tumor proliferation, angiogenesis, metastasis, medication resistance and immune evasion. The TME consists of different types of cells which have altered gene expression in cancer, including tumor associated stromal cells (TASCs), tumor endothelial cells (TECs), and immune cells, as well as extracellular matrix components. TASCs include cancer-associated fibroblasts (CAFs), tumor associated mesenchymal stem cells (TA-MSCs), tumor associated adipocytes (TAAs), and myofibroblasts. CAFs differ from normal fibroblasts in biological characteristics and gene expression profile. TECs can acquire cytogenetic abnormalities compared with normal endothelial cells (NECs) from adjacent normal tissue and may be able to acquire resistance to drugs. TAAs exhibit tumor-associated metabolic and transcriptomic phenotype. Tumor associated immune cell population correlate with better chances for survival. TA-MSCs can change their functional and phenotype following recruitment and interaction with cancer cells. The high level of TME inter- and intra-tumor heterogeneity make it an important variable in precision oncology decisions, such as cancer drug screening.

Organoids, composed of tumor epithelial cells, are 3D in vitro culture systems that have long term genetic stability in culture, simulate the epithelial architecture of the tumor of origin and represent epithelial tumor cells heterogeneity. However, a culture system that mainly includes the cancer epithelial cells, cannot completely recapitulate the TME due to the absence of CAFs, TECs, immune cells, and non-cellular components.

A tumor “avatar”, or “mini-tumor” refers to an in-vitro model composed of tumor cells, TME cells and extracellular matrix components. The incorporation of fibroblasts and endothelial cells in a two-dimensional (2D) or a 3D mini-tumor models, have been previously described (e.g., Teixeira F C et al. “Engineering a Vascularized 3D Hybrid System to Model Tumor-Stroma Interactions in Breast Cancer.” Front Bioeng Biotechnol. 2021; 9:647031, hereby incorporated by reference in its entity). Nonetheless, these cells were not derived from a TME niche, thus did not exhibit the phenotypic and the molecular features that specifically characterize the TME cells, and therefore could not accurately mimic the cross-talk between TME cells and tumor cells.

There is a great need for new methods of developing three-dimensional (3D) in vitro models of cancer, that would include TME cells, in addition to cancer cells, for mimicking the TME-tumor interaction. An in vitro model that recapitulates the intra- and inter-phenotypic and/or molecular differences between patients, not only of the tumor cells but also of the tumor niche, would allow to augment the sensitivity of cancer drugs screening, to serve as a research model for better understanding of cancer disease mechanisms, as well as to facilitate highly precise personalized onco-medicine.

### SUMMARY OF THE INVENTION

The present invention provides three-dimensional mini-tumors comprising an artificial scaffold, tumor cells, tumor associated endothelial cells and tumor associated stromal cells. Kits comprising the 3D mini-tumors, methods of producing the 3D mini-tumors and methods of using the 3D mini-tumors are also provided.

According to a first aspect, there is provided a three-dimensional (3D) tumor avatar comprising: an artificial scaffold, tumor cells, and tumor microenvironmental (TME) cells comprising tumor associated endothelial cells (TECs) and cancer associated fibroblasts (CAFs).

According to some embodiments, the TME cells are derived from a tumor tissue or a surrounding tissue thereof.

According to some embodiments, the TME cells and the tumor cells belong to a single histologic type of the tumor.

According to some embodiments, the histologic type is carcinoma or sarcoma.

According to some embodiments, the tumor cells are tumor epithelial or tumor cells in epithelial-mesenchymal transition state or tumor mesenchymal cells.

According to some embodiments, the TME cells and the tumor cells belong to a single primary or metastatic site of the tumor.

According to some embodiments, the primary site of the tumor is selected from the group consisting of: gastrointestinal cancer, kidney cancer, liver cancer, breast cancer, bladder cancer, prostate cancer, ovary cancer, uterine cancer, head and neck cancer, thyroid cancer, glioma, neuroblastoma, melanoma, and lung cancer.

According to some embodiments, the 3D tumor avatar is a patient-personalized tumor avatar, and wherein the TECs and the CAFs are present in the patient-personalized tumor avatar at a ratio found in a sample obtained or derived from the patient.

According to some embodiments, the TECs and the CAFs are present in the 3D tumor avatar culture at a ratio ranging from 1:10 to 1:1.

According to some embodiments, the ratio between tumor cells and CAFs in the 3D tumor avatar is between 1:10 and 1:1, the ratio between tumor cells and TECs in the 3D tumor avatar is between 1:10 and 1:1, or both.

According to some embodiments, the artificial scaffold comprises Matrigel, vitronectin, fibronectin, or any combination thereof.

According to some embodiments, the artificial scaffold is a Matrigel dome.

According to some embodiments, the Matrigel dome comprises a Matrigel concentration of at least 20%.

According to some embodiments, the artificial scaffold comprises a concentration gradient of Matrigel, fibronectin, vitronectin or any combination thereof, and wherein the gradient of Matrigel is from 1-100% concentration, the gradient of fibronectin is from 0-50 ng/ul and the gradient of vitronectin is from 0-50 ng/ul.

According to some embodiments, the CAFs comprise inflammatory CAFs (iCAFs) and extracellular matrix-remodeling CAFs (eCAFs).

According to some embodiments, the 3D tumor avatar further comprises tumor associated mesenchymal cells (TAMCs).

According to some embodiments, the TAMCs are tumor associated mesenchymal stem cells (TA-MSCs).

According to some embodiments, the 3D tumor avatar further comprises peripheral blood lymphocytes (PBLs).

According to some embodiments, the 3D tumor avatar further comprises tumor associated adipocytes (TAAs), tumor associated immune cells, or both.

According to some embodiments, the tumor cells are primary cells from a subject and the TME cells are also primary cells from the subject.

According to some embodiments, the tumor cells are primary cells from a subject and the TME cells are from TME cell lines.

According to some embodiments, the tumor cells are primary cells from a subject, the CAFs are primary cells from the subject and the TECs are from a TEC cell line.

According to another aspect, there is provided a kit comprising a 3D tumor avatar of the invention and a liquid media suitable for in vitro organization and maintenance of the 3D tumor avatar.

According to some embodiments, the media comprises at least one component selected from the group consisting of: hepes, abam, glutamax, non-essential amino acids, sodium pyruvate, R-Spondin, noggin, insulin growth factor 1 (IGF1), fibroblast growth factor 2 (FGF2), FGF10, epidermal growth factor (EGF), vascular endothelial ascorbic acid, growth factor (VEGF), Platelet-derived growth factor (PDGF), interleukin 6 (IL-6), IL-8, BMP4, BMP7, heparin, hydrocortisone, trombospondin, indomethacine, 3-Isobutyl-1-methylxanthine (IBMX), intralipids, bone morphogenetic protein 7 (BMP7), B27, A 83-01, N-Acetyl-L-cysteine, nicotinamide, and Y-27632.

According to some embodiments, the media comprises a plurality of components selected from the group consisting of: hepes, abam, glutamax, non-essential amino acids, sodium pyruvate, R-Spondin, noggin, IGF1, FGF2, FGF10, EGF, VEGF, PDGF, IL-6, IL-8, heparin, indomethacine, IBMX, intralipids, BMP4, BMP7, B27, N-Acetyl-L-cysteine, A 83-01, nicotinamide, and Y-27632.

According to some embodiments, the liquid media comprises DMEM/F12, DMEM/F12:ECM:MSCM:ECM (1-10:1:1:1) or ECM:MSCM:ECM (1:1:1) media.

According to some embodiments, at least one of:
a. the tumor cells are derived from a gastrointestinal cancer, and the media further comprises at least one component selected from the group consisting of: Wnt3a, gastrin, HGF and prostaglandin E2;
b. the tumor cells are derived from a kidney cancer, and the media further comprises at least one component selected from the group consisting of: HGF, epinephrine, hydrocortisone and FGF8;
c. the tumor cells are derived from a lung cancer, and the media further comprises FGF7;
d. the tumor cells are derived from a bladder cancer, and the media further comprises FGF7, heregulin or both; and
e. the tumor cells are derived from a breast, ovary or uterine cancer, and the media further comprises at least one component selected from the group consisting of: β-Estradiol, hydrocortisone, and heregulin.

According to another aspect, there is provided a method for producing a 3D tumor avatar, the method comprising:
a) providing tumor cells and tumor microenvironmental (TME) cells comprising tumor associated endothelial cells (TECs) and cancer associated fibroblasts (CAFs);
a) culturing the tumor cells and the TME cells with an artificial scaffold in a liquid media suitable for in vitro organization and maintenance of the 3D tumor avatar; thereby producing a 3D tumor avatar.

According to some embodiments, the method further comprises before step (a): isolating the tumor cells, the TME cells, or both, from a tumor tissue or a surrounding tissue thereof.

According to some embodiments, the providing TME cells comprises receiving isolated stromal cells from a tumor or a surrounding tissue thereof and culturing a first portion of the received isolated stromal cells in endothelial cell media (ECM) and culturing a second portion of the received isolated stromal cells in fibroblast medium (FM).

According to some embodiments, the culturing in ECM is for a sufficient time to enrich for iCAFs and wherein the culturing in FM is for a sufficient time to enrich for eCAFs, optionally wherein the culturing the first portion and culturing the second portion is for 1-3 weeks.

According to some embodiments, the providing TME cells comprises culturing a third portion of the received isolated stromal cells in mesenchymal stem cell medium (MSCM) and/or culturing a fourth portion of the received isolated stromal cells in MSCM:ECM:FM (1:1:1).

According to some embodiments, the providing CAFs cells comprises receiving isolated stromal cells from a tumor or a surrounding tissue thereof and culturing the received isolated stromal cells in FM and the providing TECs comprises providing cells from a TEC cell line, optionally wherein culturing in FM is for 1-3 weeks

According to some embodiments, the received isolated stromal cells are first cultured in mesenchymal stem cell medium (MSCM) before culturing the first portion, the second portion the third portion or the fourth portion, optionally wherein the culturing in MSCM is for 2-3 weeks.

According to some embodiments, the providing tumor cells comprises receiving tumor cells and culturing the tumor cells into organoids and wherein in step (b) the organoid cells are cultured with the TME cells.

According to some embodiments, the culturing of step (b) is in a media comprising at least one component selected from the group consisting of: hepes, abam, glutamax, non-essential amino acids, sodium pyruvate, R-Spondin, noggin, IGF1, FGF2, FGF10, EGF, VEGF, PDGF, IL-6, IL-8, heparin, indomethacine, IBMX, intralipids, hydrocortisone, BMP7, B27, A 83-01, N-Acetyl-L-cysteine, nicotinamide, Y-27632, Matrigel, fibronectin and vitronectin.

According to some embodiments, the media in step (b) comprises a plurality of components selected from the group consisting of: hepes, abam, glutamax, non-essential amino acids, sodium pyruvate, R-Spondin, noggin, IGF1, FGF2, FGF10, EGF, VEGF, PDGF, IL-6, IL-8, heparin, indomethacine, hydrocortisone, IBMX, intralipids, BMP7, B27, A 83-01, N-Acetyl-L-cysteine, nicotinamide, Y-27632, Matrigel and fibronectin.

According to some embodiments, the culturing of step (b) is in liquid media comprising
a. DMEM/F12, ECM:FM-2:MSCM (1:1:1) or DMEM/F12:ECM:MSCM:FM-2 (1-10:1:1:1); and
b. Hepes, ABAM, 2 GlutaMax supplement, R-spondin 1, Noggin, Wnt3a, epidermal growth factor (EGF), Gastrin, fibroblast growth factor 2 (FGF2), FGF10, gastrin, prostaglandin E2, A83-01, nicotinamide, and Vascular endothelial growth factor (VEGF), interleukin-6 (IL-6), IL-8, Bone Morphogenetic Protein 4 (BMP4) and BMP7 to produce the organoids.

According to some embodiments, the liquid media further comprises Y-27632, SB202190, trombospondin and hydrocortisone.

According to some embodiments, the method further comprises providing immune cells and culturing the immune cells with the tumor cells and the TME cells in step (b).

According to some embodiments, the liquid media further comprises IL-2, beta-mercaptoethanol, IL-4, IL-13 and Macrophage colony-stimulating factor (M-CSF).

According to some embodiments, the tumor is selected from the group consisting of: gastrointestinal cancer, kidney cancer, liver cancer, breast cancer, bladder cancer, ovary cancer, uterine cancer, lung cancer, and any combination thereof.

According to some embodiments, the tumor is a gastrointestinal cancer and the culturing is in a media comprising at least one component selected from the group consisting of: Wnt3a, gastrin, and prostaglandin E2.

According to some embodiments, at least one of:
a. the tumor is a kidney cancer, and the culturing is in a media comprising at least one component selected from the group consisting of: hydrocortisone, epinephrine, FGF8 and HGF;
b. the tumor is a lung cancer, and the culturing is in a media comprising FGF7;
c. the tumor is a bladder cancer, and the culturing is in a media comprising heregulin, FGF7 or both; and
d. the tumor is a breast, ovary or uterine cancer, and the culturing is in a media comprising at least one component selected from the group consisting of: β-Estradiol, hydrocortisone and heregulin.

According to some embodiments, the culturing in step (b) is between 1 day and two months.

According to some embodiments, the artificial scaffold comprises Matrigel, vitronectin, fibronectin, or any combination thereof.

According to some embodiments, the artificial scaffold is a Matrigel dome.

According to some embodiments, the Matrigel dome comprises a Matrigel concentration of at least 20%.

According to some embodiments, the artificial scaffold comprises a concentration gradient of Matrigel, fibronectin, vitronectin or any combination thereof, and wherein the gradient of Matrigel is from 1-100% concentration, the gradient of fibronectin is from 0-50 ng/ul and the gradient of vitronectin is from 0-50 ng/ul.

According to some embodiments, the method further comprises before step (a):
i) receiving a tumor sample from a subject afflicted with the cancer, wherein the sample comprises tumor cells, TECs and CAFs; and,
ii) determining the ratio between the tumor cells and the TME cells, and between the TECs and the CAFs in the sample;
wherein the culturing the tumor cells and the TME cells is according to the determined ratio in the sample, and wherein the culturing ratio is up to +20% of the determined ratio.

According to another aspect, there is provided a 3D tumor avatar produced by a method of the invention.

According to another aspect, there is provided a method for screening a drug for being suitable as an anticancer drug, the method comprising:
a) culturing a 3D tumor avatar of the invention in a culture media,
b) contacting the 3D tumor avatar with the drug; and,
c) measuring an anticancer effect in the 3D tumor avatar, wherein presence of the effect above a predetermined threshold indicates the drug is suitable for being an anticancer drug;
thereby screening a drug or combination of drugs for being suitable as an anticancer drug.

According to some embodiments, the anticancer effect is reduced cell viability, reduced proliferation rate, or both.

According to some embodiments, the predetermined threshold is the presence of the anticancer effect in the 3D tumor avatar culture without a drug or contacted with a control drug.

According to some embodiments, the method is a method of personalized anticancer drug selection for a subject suffering from cancer and the 3D tumor avatar comprises cancer cells derived from the subject.

According to some embodiments, the TECs and CAFs are derived from the cancer of the subject.

According to some embodiments, before step (a) the drug is selected by a method comprising:
a. receiving transcriptomic data from the tumor cells and the TME cells;
b. identifying at least one gene upregulated in the tumor cells as compared to the TME cells; and
c. selecting a drug that inhibits the at least one upregulated gene or its protein product.

According to some embodiments, the method further comprises producing a Strengths, Weaknesses, Opportunities, and Threats (SWOT) analysis of the drug as an anticancer therapeutic for a subject that provided the tumor cells.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1J: Characterization of Patient-Derived Gastric Tumor Cells in Culture. (1A) Overview of the experimental plan illustrating the culture and analysis of gastric tumor-derived cells. (1B) Representative bright field images comparing organoids cultured in distinct mediums (M1-4) over 6 days, (1C) and organoids cultured in medium M3 at days 0, 1, 2, and 6. (1D-1E) Boxplots depicting variations in organoid size and number in their culture (1D) across mediums MI to M4 and (1E) during 6 days in M3. Bar=300 μM. (1F) Representative brightfield and immunofluorescence images of organoid slices co-stained with ALDH1, CD133, E-CDH, CD73, CD105 and counterstain with Hoechst (blue). Bar=50 μM (1G-1I) Representative brightfield and immunofluorescence images of tumor-derived cells cultured in (1G) MSCM-FM2, (1H) MSCM-ECM or (1I) MSCM co-stained with Vimentin (red), αSMA (green), MCAM (purple); CD44 (red), CD73 (green) and CD105 (purple) and counterstain with Hoechst (blue). Bar=100 μM (1J) Quantification of different markers in cultured cells through flow cytometry analysis. Results show mean±SE of three biological replicates, ** P<0.05, **** p<0.005.

FIGS. 2A-2H: Comparative Genomic Landscape of Gastric Tumor Paired Epithelial and Stroma Cultured Cells. (2A) Venn Diagrams illustrating the number of SNPs, insertions, and deletions observed in the parental tumor compared to the corresponding tumor-derived organoids and cells cultured in ECM, FM-2, and MSCM mediums. The data is presented before (left) and after filtering for mutated genes present in the normal sample (right), with allele frequencies >0.05, or with low mutation impact. (2B) Oncoplot displaying the distribution of gastric adenocarcinoma associated somatic mutations of the top mutated genes in tumor tissue compared with tumor-derived cultured cells. The bar chart above the oncoplot indicates the proportion of different mutation types in each sample. The color of the square denotes the type of mutation detected: missense (dark green), frameshift deletion (blue), frameshift insertion (violet), in frame deletion (dark yellow), in frame insertion (red), nonsense (light blue), nonstop (pink), splice site (orange), translation start site (brown), multi hot (black). (2C) Reactome and KEEG enrichment analysis of canonical pathways affected by germline mutations or by somatic mutations in: (2D) Tumor, (2E) Organoids, (2F) MSCM-FM2, (2G) MSCM-ECM and (2H) MSCM mediums tumor-derived cultured cells.

FIGS. 3A-3K. Gene Expression Analysis of Gastric Tumor Paired Organoids and Stroma Cultured Cells. (3A) Hierarchical Clustering Heatmap illustrating differentially expressed genes with statistical significance and fold change among organoids and tumor derived stroma cells cultured in FM2, ECM and MSCM mediums. (3B-3C) Lolliplot charts depicting (3B) Reactome and KEEG enriched signaling in organoids compared to cultured stroma cells and (3C) significantly upregulated signaling pathways in stroma cells compared to organoids. (3D) Hierarchical Clustering Heatmaps displaying differentially expressed oncogenes with statistical significance and fold change between organoids and cultured stroma cells. (3E) Hierarchical Clustering Heatmaps demonstrating differentially expressed epithelial and mesenchymal cell type markers. (3F) Hierarchical Clustering Heatmaps showing differentially expressed genes with statistical significance and fold change between FM2, ECM and MSCM stroma cells cultures. (3G) Volcano plot depicting differentially expressed genes with statistical significance and fold change among cells cultured in MSCM and FM2 mediums, FM2 and ECM mediums, and MSCM and ECM mediums. Each dot represents a gene, colored by adjusted p-value as per the legend. (3H-3I) Lolliplot charts presenting Reactome and KEEG enriched signaling in cells cultured in FM2 medium compared to cells cultured in (3H) MSCM and (3I) ECM mediums. (3J-3K) Lolliplot charts showing Reactome and KEEG enriched signaling in cells cultured in (3J) MSCM medium or (3K) ECM as compared to cells cultured in FM2 medium.

FIGS. 4A-4K. Development of a Gastric Adenocarcinoma Co-Culture Model: Integrating Organoids and Cancer-Associated Stromal Cells. (4A-4F) Cells cultured in ECM and HUVEC cells were stained with CellTrace Violet Dye (blue cells), tumor derived stroma cells cultured in FM-2 medium were stained with CellTrace CFSE Dye (green cells) and ORG were stained with CellVue Claret far red fluorochrome (purple cells) before co-culturing. The total cell count remained consistent across co-cultures and organoids. The cells were mixed and co-cultured in 384 well low attachment flat plates. Pictures were taken at day 5 of culture. Representative brightfield images and whole Z-stacks immunofluorescence staining of co-cultures composed of: (4A) Tumor associated stromal cells cultured in ECM or FM2 mediums, and gastric cancer organoids mixed at ratios of 1:1:1, 1:4:1, 4:1:1 or 1:1:4, respectively; (4B) HUVEC endothelial cells, tumor associated stromal cells cultured in FM2 medium and organoids mixed at the same ratios; (4C) Tumor associated stromal cells cultured in FM2 medium and organoids mixed at ratios of 1:1, 1:4 or 4:1, respectively; (4D) Tumor associated stromal cells cultured in ECM medium and organoids mixed at ratios of 1:1, 1:4 or 4:1, respectively; (4E) HUVEC and ORG mix at ratios of 1:1, 1:4 or 4:1, respectively; (4F) Organoids monoculture. (4G-4K) Cell growth kinetics of tumor-derived cell co-cultures are depicted as line graphs showing cell viability compared to organoids. The viability of the co-cultures was tested using CellTiter-Glo assay to measure ATP, a biomarker of cell health. The assay was performed on days 3, 5, and 10 of the culture. (4G) Cell viability of ECM:FM2:ORG co-cultures at ratios of 1:1:1, 1:4:1, 4:1:1 or 1:1:4 respectively, compared to organoids; (4H) HUVEC:FM2:ORG co-cultures at ratios of 1:1:1, 1:4:1, 4:1:1 or 1:1:4 respectively, compared to organoids; (4I) FM2:ORG co-cultures at ratios of 1:1, 1:4 or 4:1 respectively, compared to organoids; (4J) ECM:ORG co-cultures at ratios of 1:1, 1:4 or 4:1 respectively, compared to organoids; and (4K) HUVEC:ORG co-cultures at ratios of 1:1, 1:4 or 4:1 respectively, compared to organoids. Results show mean±SE. Scale bar: 200 um.

FIGS. 5A-5E. Impact of Various Scaffolds on Spatial Distribution in Co-Culture Systems. Cells cultured in ECM and Huvec cells were stained with CellTrace Violet Dye (blue cells), ORG cells were stained with CellTrace CFSE Dye (green cells), and tumor derived stroma cells cultured in FM-2 medium were stained with CellVue Claret far red fluorochrome (purple cells) before co-culturing. The total cell count remained consistent across co-cultures and organoids. Co-cultures were plated in 384-well low-attachment flat plates without scaffold (no Matrigel, no fibronectin), with a 5% Matrigel scaffold, or with both a 5% Matrigel and 10 ng/ul fibronectin scaffold over 7 days. Representative brightfield images and whole Z-stack immunofluorescence illustrate co-cultures: (5A) Tumor associated stromal cells cultured in ECM or FM2 mediums, and gastric cancer organoids mixed at ratios of 1:1:1, 1:4:1, 4:1:1 or 1:1:4, respectively; (5B) HUVEC endothelial cells, tumor associated stromal cells cultured in FM2 medium and organoids mixed at the same ratios; (5C) Tumor associated stromal cells cultured in FM2 medium and organoids mixed at ratios of 1:1, 1:4 or 4:1, respectively; (5D) Tumor associated stromal cells cultured in ECM medium and organoids mixed at ratios of 1:1, 1:4 or 4:1, respectively; (5E) HUVEC and ORG mix at ratios of 1:4 or 4:1, respectively. Scale bar 40 um.

FIGS. 6A-6D. Exploring Epithelial and Stromal Cell Marker Expression and Localization in Co-Culture Systems. (6A) Representative Hematoxylin and Eosin (H&E) images of ECM:FM-2:ORG and Huvec:FM-2:ORG co-culture paraffin sections at ratios of 1:1:1, 1:4:1, 4:1:1, and 1:1:4 respectively compared with organoids and primary tumor tissue from the same sample. (6B-6D) Images were captured using a Leica Microscope. Representative whole Z-stack immunofluorescence images of: (6B) primary tumor tissue and organoids, (6C) ECM:FM-2:ORG and (6D) Huvec:FM-2:ORG co-culture paraffin sections from the same sample, illustrating the expression and localization of Vimentin (Red), EPCAM (Gray), and MCAM (Green), with Hoechst counterstain (blue) at ratios of 1:1:1, 1:4:1, 4:1:1, and 1:1:4 respectively. Scale bar=100 μM. Images were captured using a Zeiss Confocal Microscope at 20× and 40× magnification.

FIGS. 7A-7J. Gene Expression Analysis of Gastric Adenocarcinoma Organoids and Cancer-Associated Stromal Cells Co-Culture Models. (7A) Volcano plot displaying genes with significant differential expression in ECM:FM-2:ORG and Huvec:FM-2:ORG co-culture at equivalent cell ratios, Huvec:FM-2:ORG and ORG, and ECM:FM-2:ORG and ORG. Each dot represents a gene, colored by adjusted p-value as per the legend. (7B) Hierarchical Clustering Heatmap illustrating differentially expressed genes with statistical significance and fold change among organoids and co-cultures:HUVEC:FM-2:ORG at ratios of 1:1:1, 1:4:1, 4:1:1, and 1:1:4 respectively depicted as CO-CULT1-4; ECM:FM-2:ORG at ratios of 1:1:1, 1:4:1, 4:1:1, and 1:1:4 respectively depicted as CO-CULT5-8. (7C) Lolliplot chart depicting enriched signaling pathways in co-cultures compared to organoids. (7D) Significantly upregulated signaling pathways in ECM:FM-2:ORG co-cultures compared to organoids and cultured stroma cells. (7E) Significantly upregulated signaling pathways in HUVEC:FM-2:ORG co-cultures compared to organoids and stroma cells. (7F) Hierarchical Clustering Heatmaps displaying differentially expressed genes with statistical significance and fold change between ECM:FM-2:ORG and HUVEC:FM-2:ORG co-cultures. (7G) Lolliplot charts presenting enriched signaling pathways in ECM:FM-2:ORG co-cultures compared to HUVEC:FM-2:ORG co-cultures. (7H) Lolliplot charts showing enriched signaling pathways in HUVEC:FM-2:ORG co-cultures compared to ECM:FM-2:ORG co-cultures. (7I) Hierarchical Clustering Heatmaps demonstrating differentially expressed epithelial and mesenchymal cell type markers in the co-cultures compared to organoids. (7J) Hierarchical Clustering Heatmap Illustrating Differential Gene Expression in Co-Cultures Compared to Organoids and Cells Cultured in MSCM, ECM, and FM2, highlighting genes with elevated expression levels or induction specifically in co-culture conditions.

FIGS. 8A-8I. Influence of Stroma Cell Presence in Co-Culture on Drug Responsiveness. (8A) Co-cultures of ECM:FM-2:ORG and HUVEC:FM-2:ORG at ratios of 1:1:1, 1:4:1, 4:1:1, or 1:1:4, along with organoids, were treated for 72 hours with paclitaxel. Representative bright field images are provided. Images were acquired with Operetta CLS Imager Microscope at 20× magnification. (8B) Cell viability was assessed using the CellTiter-Glo assay. A bar graph presents a summary of cell viability results. Results show mean±SE (n=6). Statistical significance indicated as *P<0.05, ** P<0.01, **** P<0.001. (8C) Co-cultures of ECM:FM-2:ORG and HUVEC:FM-2:ORG at ratios of 1:1:1, 1:4:1, 4:1:1, or 1:1:4, along with organoids, were treated for 72 hours with fluorouracil, Leucovorin, Oxaliplatin, and Docetaxel (FLOT). Representative bright field images are provided. Images were acquired with Operetta CLS Imager Microscope at 20× magnification. (8D) Heatmap illustrating PanDrugs-selected targeted drugs with a drug score exceeding 0.7, derived from differentially expressed genes between ECM:FM-2:ORG co-cultures and organoids, along with stroma cell monocultures. DScore: PanDrugs drug score; GScore: PanDrugs gene score. (8E) Heatmap depicting the gene count associated with each PanDrugs-selected targeted drug across a specific range of constant inhibition values, as determined by DrugTargetCommons and PubChem databases. (8F) Heatmap showing ORG Score, representing the normalized expression level of genes related to each selected drug in organoids, Fold Change (FC) score resulting from the normalized fold change expression of these genes in the organoids relative to stroma cells, and Evidence Score ranking the genes based on published data linking them with gastric cancer oncogenesis and drug response. (8G) Cell viability assay was conducted to assess the efficacy of selected target drugs in ECM:FM2:ORG co-cultures compared to organoids and stroma cell monocultures. Results show mean±SE (n=6). Statistical significance indicated as *P<0.05, ** P<0.01, *** P<0.005, **** P<0.001. (8H) Representative bright field images comparing ECM:FM2:ORG co-cultures, organoids, and stroma cell monocultures after treatment for 72 hours with tested targeted drugs. (8I) Prioritization of tested drugs using Strengths, Weaknesses, Opportunities, Threats (SWOT) analysis.

FIGS. 9A-9B: (9A) Representative brightfield images of human kidney tumor avatar composed of Huvec endothelial cells, Org and TASCs mix at a 1:4:4, 1:1:1 or 4:1:4 ratio, respectively cultivated in a Matrigel (5%) and fibronectin scaffold (10 ng/ml), at day 0 and 6 of culture. (9B) IF staining is presented for a TASCs marker: a-SMA; a stem cell marker: CD44; and a tumor epithelial cell marker: cadherin E (CDHE). Images were acquired with Zeiss Confocal Microscope at 10 and 60× magnifications. Scale bar: 40 um

FIGS. 10A-10B: (10A) Representative immunofluorescence and brightfield images of human pancreas tumor avatar composed of Huvec endothelial cells, Org and TASCs mix at a 1:1:1, 1:4:1 or 4:1:1 ratio, respectively cultivated in a Matrigel (5%) and fibronectin scaffold (10 ng/ml), during 6 days. Org were stained with Cell Vue Claret far red fluorochrome (purple cells). Cell viability was assessed by Calcein (green)/PI (red) staining and images were acquired with Operetta Imager at 5× magnification. (10B) Line graphs showing relative light unit percentage (RLU %) of everolimus or FOLFOX treated pancreas tumor avatars and organoids as compared with their respective vehicle controls, after the same day of drug or vehicle exposure, (RLU %=RLUtreatment*100)/RLUveh). Tumor avatars were cultured for 3 days, treated with the specified drugs for other 72 hours and viability determined by Celltiter-Glo assay.

FIG. 11: Representative brightfield images of human lung tumor avatar composed of Org and TASCs mix at a 10:1 and 20:1 ratio, respectively cultivated in a Matrigel (5%), at day 2, 4 and 7 of culture.

FIGS. 12A-12F: (12A) Representative brightfield images of human gastric tumor avatars from a patient with gastrointestinal sarcoma (patient 1) and a second patient (patient 2) with gastric adenocarcinoma, composed of organoids and stroma cells. Cells were dissociated from gastric tissue and cultured in a Matrigel dome in an adherent plate in tumor avatar medium from day 0. After 7 days of culture, organoids remain in the dome and stroma cells and metastatic tumor cells invaded the tissue culture plate outside the dome. Tumor cells from patients 2 did not survive in organoids medium. (12B) Representative brightfield images of defrosted human gastric tumor avatars from defrosted tumor avatars. The tumor avatars were sub-cultured in a Matrigel dome using an adherent plate in the presence of tumor avatar medium from day 3 to day 13. Subculture tumor avatars showed similar cells distribution to the tumor avatar cultured in similar conditions after tissue dissociation. (12C-12D). Representative brightfield images of human gastric tumor avatars from a patient with gastrointestinal sarcoma (patient 1) and a second patient (patient 2) with gastric adenocarcinoma, composed of organoids and stroma cells. Cells were dissociated from gastric tissue and cultured in a low repellent plate in tumor avatar medium from day 0 to day 35. A magnification course is shown in 12C. A time course is shown in 12D. Calcein-PI-DAPI images are shown in 12E. (12F) Cells were dissociated from gastric tissue and cultured in a low repellent plate in tumor avatar medium. At the day of treatment cultured lymphocytes from the peripheral blood of the same patient (n=1000) were added to the tumor avatar. Then, the tumor avatars in the presence of lymphocytes were treated with different concentrations of Pembrolizumab immunotherapy. Cell viability was detected after 72 hours of treatment using Cell-Titer Glo assay.

FIGS. 13A-13B: Representative brightfield and immunofluorescence images of human gastric tumor avatars from (13A) a patient with gastrointestinal sarcoma (patient 1) and (13B) a second patient (patient 2) with gastric adenocarcinoma, composed of organoids and stroma cells. Cells were dissociated from gastric tissue and cultured in a matrigel dome in a non-repellent plate in tumor avatar medium from day 0. After 7 days of culture, organoids remains in the dome and stroma cells and metastatic tumor cells invaded the tissue culture plate outside the dome. Tumor cells from patients 2 did not survive in organoids medium. Cells were co-stained with Vimentin (red), MCAM (green or purple) and EPCAM (purple or green), CD90 (red), CD73 (green), and CD105 (purple), and counterstained with Hoechst (blue). The staining of each row is listed below, and annotation is as follows: row 1/row 2/row 3. The presence of stroma cells was denoted by Vimentin and MCAM, whereas tumor epithelial cells showed EPCAM expression. Furthermore, a subpopulation of stem cells stained with CD105 and CD73 was also observed. EMT were positive for both Vimentin and EPCAM stains.

FIGS. 14A-14C: (14A-14B) Representative (14A) brightfield and (14B) immunofluorescence images of gastric cancer organoids cultured on domes prepared with different Matrigel concentrations (80%, 60%, 40% and 20%). Organoids were seeded on previously formed domes, incubated for 30 or 60 minutes and cultured in tumor avatar medium. At higher concentrations organoids remained outside the dome. Domes with Matrigel concentration lower of 40% or lower allowed the organoids to grow on the domes. Organoids were stained with CellTrace™ CFSE Dye (green cells). (14C) Representative brightfield images of gastric cancer organoids resuspended in different Matrigel concentrations (60%, 40% and 20%) and seeded to form a dome. After 30 min of incubation, growth medium was added to each well. Cells were cultured during 11 days in organoid growth medium. Scale bar 2550 um.

FIGS. 15A-15B: (15A) Representative brightfield images of gastric tumor stromal cells added to a previously formed dome with various Matrigel concentrations (80%, 60%, 40% and 20%). After a 30 minute incubation, stromal cells were cultured for 11 days in stroma cells medium (ECM:MSCM:FM 1:1:1). Stromal cells consisted of a mix of patient derived tumor cells cultured in ECM, MSCM and FM in a 1:1:1 ratio. Matrigel concentrations higher than 20% inhibited stroma cell growth inside or on the domes. (15B) Representative brightfield images of gastric tumor stroma cells and organoids resuspended in various concentrations of Matrigel (60%, 40% and 20%) and seeded to form a dome. After 30 min of incubation, growth medium was added to each well. Cells were cultured for 9 days in DMEM/F12+growth factors (GFs) or ECM:MSCM:FM2+GFs or tumor avatar medium. Stromal cells consisted of a mix of patient derived tumor cells cultured in ECM, MSCM and FM in a 1:1:1 ratio. It was observed that organoids remained in the dome and stromal cells moved outside the dome, toward the bottom of the well, at higher Matrigel concentrations. At Matrigel concentrations of 20%, stromal cells were observed in the dome and throughout the rest of the well.

### DETAILED DESCRIPTION OF THE INVENTION

The present invention, in some embodiments, provides three-dimensional tumor avatars comprising an artificial scaffold, tumor cells, tumor associated endothelial cells and tumor associated stromal cells. Kits comprising the 3D tumor avatars and a liquid media suitable for the 3D tumor avatar are also provided. Methods of producing the 3D tumor avatar comprising culturing tumor cells, tumor associated endothelial cells and tumor associated stromal cells are also provided. Methods of screening a drug for being an anticancer drug, comprising culturing a 3D tumor avatar in the presence of the drug and measuring an anticancer effect in the 3D tumor avatar are also provided.

By a first aspect, there is provided a tumor avatar comprising: tumor cells and tumor microenvironment (TME) cells.

In some embodiments, the tumor avatar is in vitro. In some embodiments, the tumor avatar is ex vivo. In some embodiments, the tumor avatar is not a naturally occurring tumor. In some embodiments, the tumor avatar is a not a tumor in a subject's body. In some embodiments, the tumor avatar is in culture. In some embodiments, the tumor avatar is in a dish. In some embodiments, the tumor avatar is in tissue culture.

In some embodiments, the tumor avatar is a mini-tumor. In some embodiments, the tumor avatar is representative of a tumor. In some embodiments, a tumor avatar is a tumor model. In some embodiments, the tumor avatar is a model of a tumor from a subject. In some embodiments, the tumor avatar is a patient-personalized tumor avatar. In some embodiments, the model is a patient-specific or patient-personalized model. In some embodiments, the tumor avatar is three-dimensional (3D). In some embodiments, the tumor avatar is a 3D model. In some embodiments, the tumor avatar is not a layer of cells. In some embodiments, the tumor avatar is not a spheroid of tumor cells.

In some embodiments, the tumor cells are derived from a tumor. In some embodiments, the tumor cells are primary cells. In some embodiments, the tumor cells are from a subject. In some embodiments, the tumor cells are from a cell line. In some embodiments, the tumor cells are not from a cell line. In some embodiments, the tumor cells are derived from a tumor and have been in culture for not more than 1, 2, 3, 4, 5, 6, 7, 8, 9, 10, 11, 12, 13, 14, 15, 16, 17, 18, 19 or 20 passages. Each possibility represents a separate embodiment of the invention.

In some embodiments, the TME cells comprise endothelial cells. In some embodiments, the endothelial cells are tumor associated endothelial cells (TECs). As used herein, the term “tumor associated” refers to cells that have existed in a subject in the TME of a cancer, i.e. were growing in proximity to a tumor and thus are tumor associated. In some embodiments, tumor associated cells are part of the tumor. In some embodiments, tumor associated cells are from tissue surrounding the tumor. In some embodiments, tumor associated cells are from the tumor and/or tissue surrounding the tumor. In some embodiments, the TME cells are stromal cells. In some embodiments, endothelial cells are stromal cells. In some embodiments, the stromal cells are tumor associated stromal cells (TASCs). In some embodiments, the stromal cells are fibroblasts. In some embodiments, the TASCs comprises cancer-associated fibroblasts (CAFs). In some embodiments, the TASCs comprises tumor associated fibroblasts (TAFs). In some embodiments, the TME cells comprises TECs and CAFs.

As used herein, the terms “CAF” and “TAF” are synonymous and used interchangeably. In some embodiments, the TASCs comprises a plurality of subpopulations of CAFs. In some embodiments, the CAFs comprises a plurality of types of CAFs. In some embodiments, the CAFs comprise inflammatory CAFs (iCAFs). In some embodiments, the CAFs comprise extracellular matrix-remodeling CAFs (eCAFs). In some embodiments, the CAFs are enriched for iCAFs. In some embodiments, the CAFs are enriched for eCAFs. In some embodiments, the CAFs have been cultured in ECM. In some embodiments, the CAFs have been cultured in FM. iCAFs and eCAFs are well known in the art and the markers that identify these cells are also known. A skilled artisan would be able to identify these cells by their proteomic and transcriptomic repertoires. iCAFs are known to have increased expression of interleukins (e.g., IL4R, IL7R, IL6, IL17, IL18 and IL27RA), and genes of the MAPK, PI3K-Akt, p53, TNF, Hippo, TGF-beta, and NOTCHI signaling pathways. In some embodiments, an iCAF expresses increased levels of at least one of IL4R, IL7R, IL6, IL17, IL18 and IL27RA. In some embodiments, increased is as compared to CAFs. In some embodiments, increased is as compared to a mixed population of CAFs from a tumor. In some embodiments, increased is as compared to eCAFs. eCAFs are known to have increased expression of cholesterol, lipids, steroids, and genes involved in sphingolipids metabolism and triglyceride metabolism. In some embodiments, an iCAF comprises increased expression of cholesterol, lipids, or steroids. In some embodiments, increased is as compared to CAFs. In some embodiments, increased is as compared to a mixed population of CAFs from a tumor. In some embodiments, increased is as compared to iCAFs.

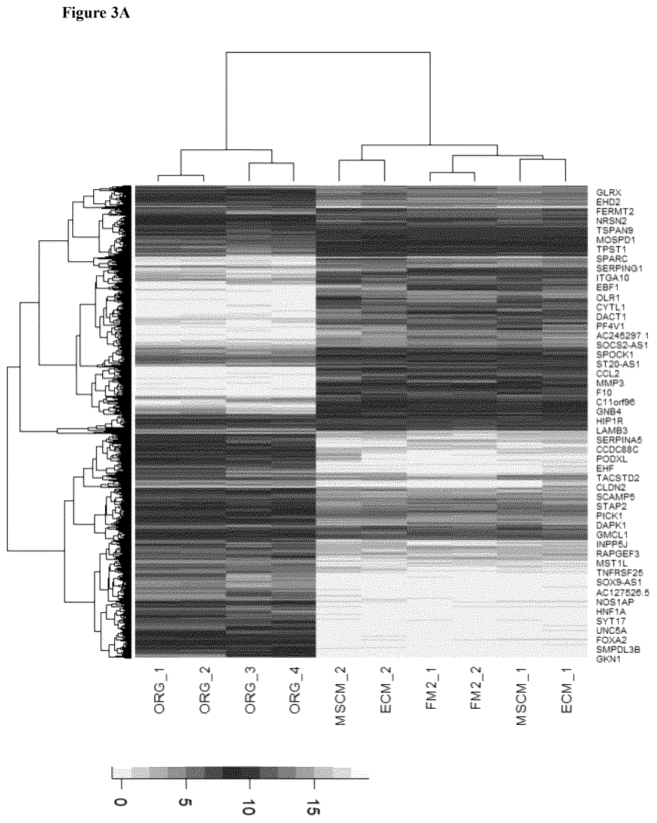

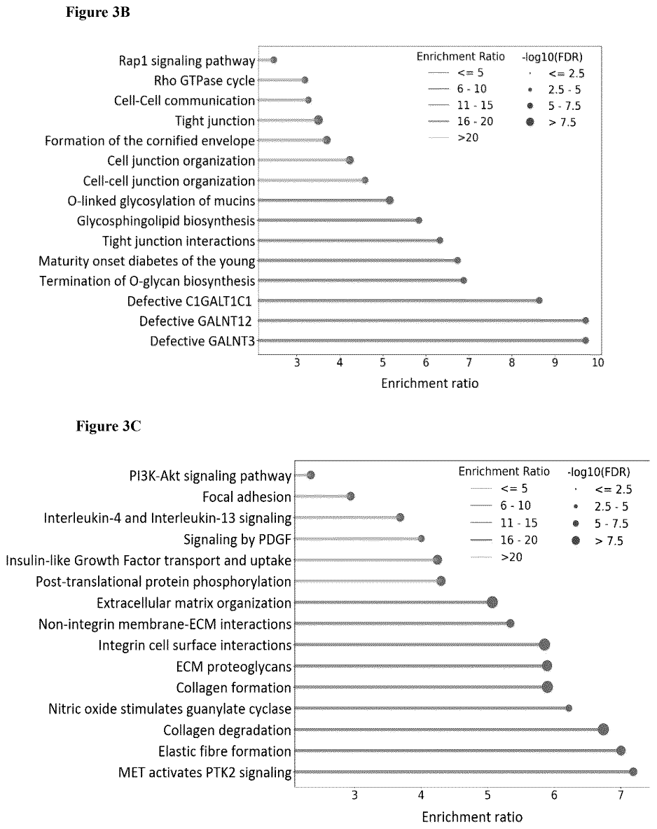

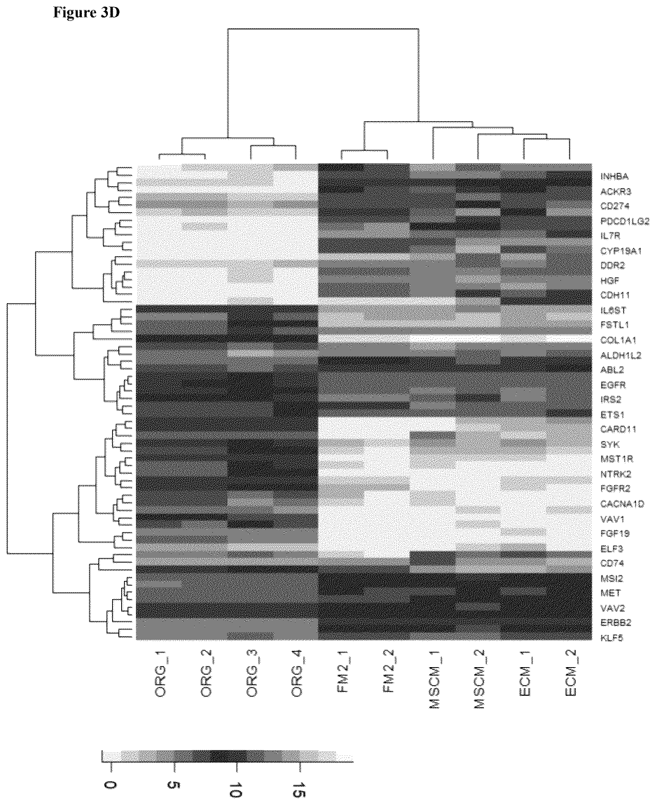

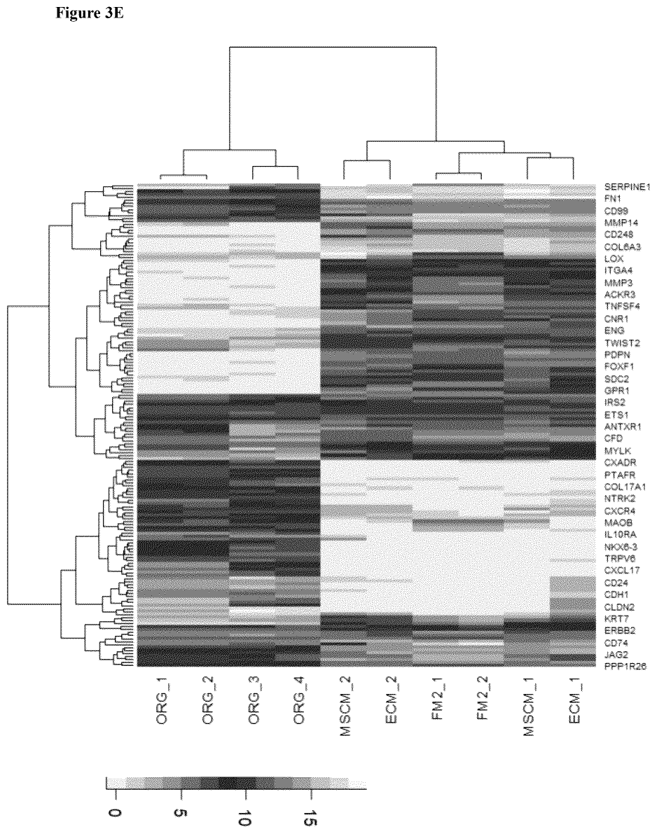

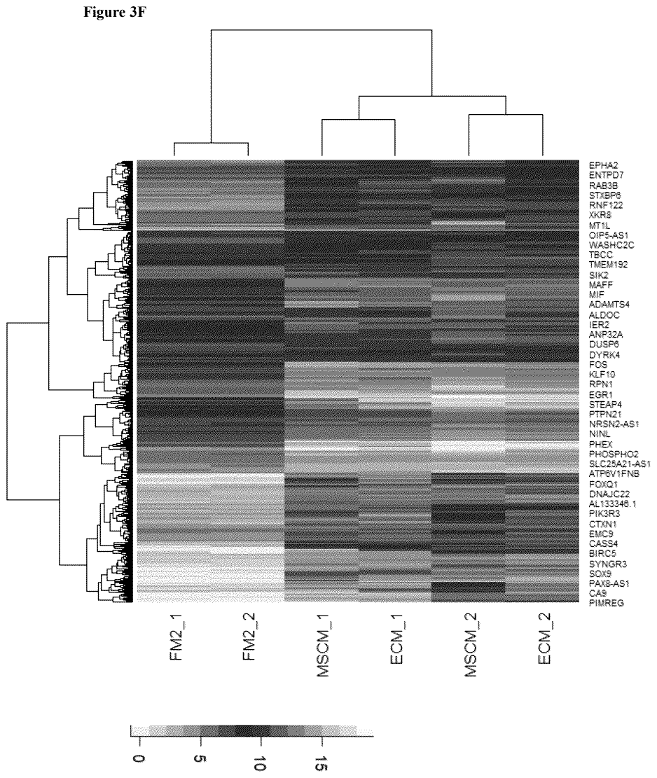

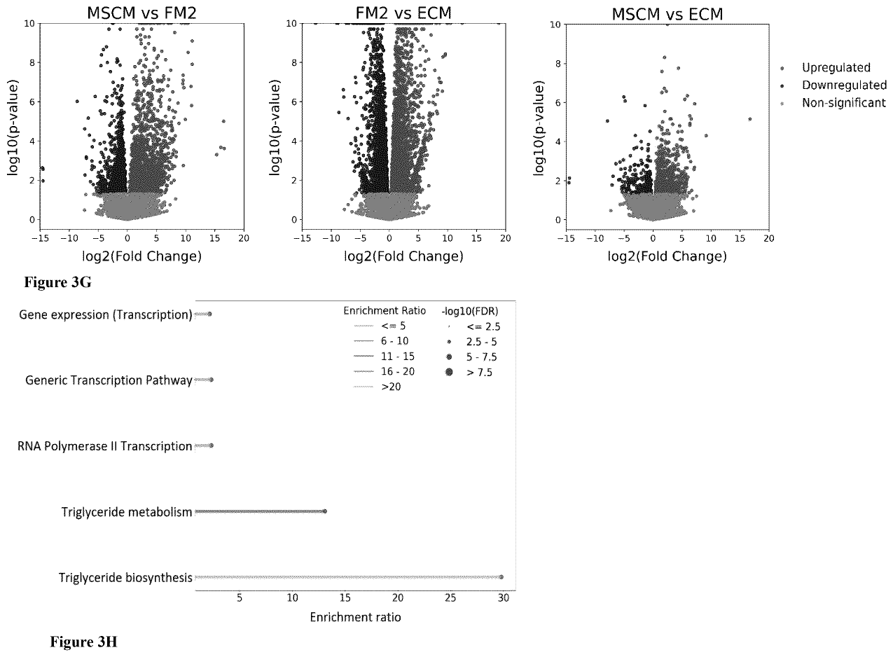

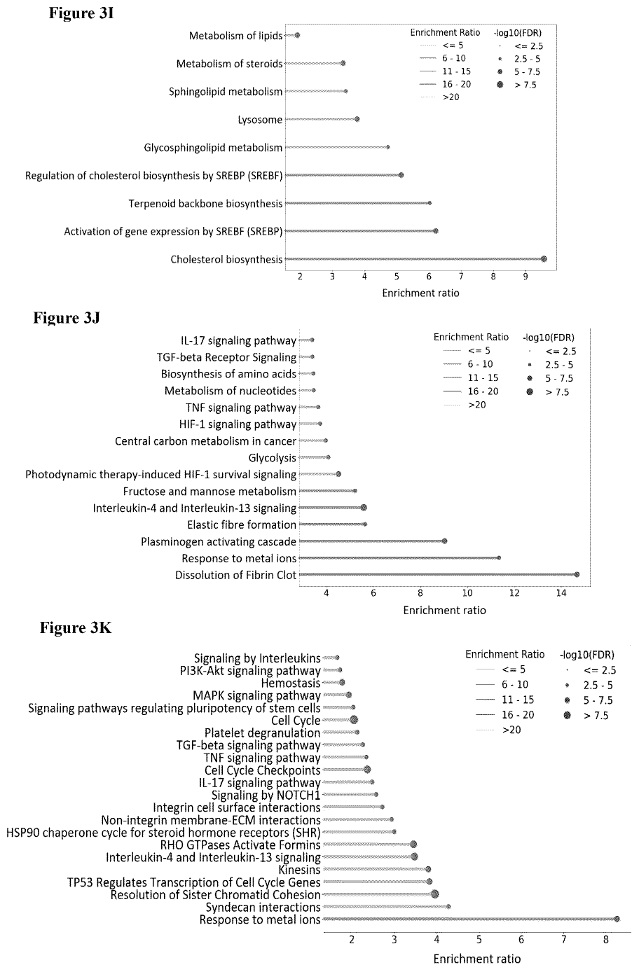

In some embodiments, markers of the different CAF subtypes are presented in FIG. 3F. In some embodiments, a first subtype comprises increased expression of at least one of: MT1L, SIK2, MAFF, MIF, ADAMTS4, ALDOC, IER2, ANP32A, DUSP6, DYRK4 and FOS. In some embodiments, a first subtype comprises increased expression of at least one of: MT1L, SIK2, MAFF, MIF, ADAMTS4, ALDOC, IER2, ANP32A, DUSP6, DYRK4, FOS, EGR1, STEAP4, PTPN21, NRSN2-AS1, NINL, PHEX, PHOSPHO2, and SLC25A21-AS1. In some embodiments, the first type is CAFs cultured in FM-2 media. In some embodiments, a second subtype comprises increased expression of at least one of: EPHA2, ENTPD7, RAB3B, STXBP6, RNF122, and XKR8. In some embodiments, the second type is CAFs cultured in ECM media. In some embodiments, a third subtype comprises increased expression of at least one of: ATP6V1FNB, FOXQ1, DNAJC22, AL133346.1, PIK3R3, CTXN1, EMC9, CASS4, BIRC5, SYNGR3, SOX9, PAX8-AS1, CA9 and PIMREG. In some embodiments, the third type is CAFs cultured in MSCM media.

In some embodiments, the TME cells are immune cells. In some embodiments, the immune cells are tumor infiltrating immune cells. In some embodiments, the immune cells are TME infiltrating immune cells. In some embodiments, the immune cells are tumor infiltrating lymphocytes (TILs). In some embodiments, the avatar further comprises immune cells. In some embodiments, the immune cells are lymphocytes. In some embodiments, the lymphocytes are peripheral blood lymphocytes (PBLs). In some embodiments, the lymphocytes are from the subject. In some embodiments, the lymphocytes are from the same subject as provided the tumor cells.

In some embodiments, the TME cells are adipose cells. In some embodiments, the adipose cells are adipocytes. In some embodiments, the adipose cells are tumor associated adipocytes (TAAs). In some embodiments, the TME cells are mesenchymal cells. In some embodiments, the stromal cells are mesenchymal cells. In some embodiments, the mesenchymal cells are tumor associated mesenchymal cells (TAMCs). In some embodiments, the mesenchymal cells are mesenchymal stem cells (MSCs). In some embodiments, the TAMCs are tumor associated mesenchymal stem cells (TA-MSCs).

In some embodiments, the TME cells comprise endothelial cells and stromal cells. In some embodiments, the TME cells comprise endothelial cells and fibroblasts. In some embodiments, the TME cells comprise endothelial cells and mesenchymal cells. In some embodiments, the TME cells comprises endothelial cells and MSCs. In some embodiments, the TME cells comprise endothelial cells, fibroblasts and mesenchymal cells. In some embodiments, the TME cells comprise endothelial cells, fibroblasts and MSCs. In some embodiments, all of the TME cells are tumor associated cells.

In some embodiments, the TME cells are derived from a tumor. In some embodiments, the TME cells are derived from tumor tissue. In some embodiments, the TME cells are derived from tissue surrounding a tumor. In some embodiments, the TME cells are derived from surrounding tissue of the tumor. In some embodiments, the TME cells are primary cells. In some embodiments, the TME cells are from a subject. In some embodiments, the subject is the same subject that the tumor cells are from. In some embodiments, the subject is the same subject as provided the tumor cells. In some embodiments, the TME cells are from a cell line. In some embodiments, the TME cells are not from a cell line. In some embodiments, the TME cells are derived from a tumor or surrounding tissue thereof and have been in culture for not more than 1, 2, 3, 4, 5, 6, 7, 8, 9, 10, 11, 12, 13, 14, 15, 16, 17, 18, 19 or 20 passages. Each possibility represents a separate embodiment of the invention.

In some embodiments, the tumor cells and TME cells belong to a single histological type. In some embodiments, histological type is a tumor type. In some embodiments, the histological type is selected from carcinoma, sarcoma, myeloma, leukemia and lymphoma. In some embodiments, the histological type is selected from carcinoma, sarcoma, myeloma, brain and spinal cord cancers, leukemia and lymphoma. In some embodiments, the histological type is selected from carcinoma, sarcoma, and myeloma. In some embodiments, the histological type is selected from carcinoma, sarcoma, myeloma and brain and spinal cord cancers. In some embodiments, the histological type is selected from carcinoma and sarcoma. In some embodiments, the histological type is selected from carcinoma, sarcoma and brain and spinal cord cancers. In some embodiments, carcinomas are selected from squamous cell carcinoma, adenocarcinoma, transitional cell carcinoma, and basal cell carcinoma. In some embodiments, sarcomas are selected from bone sarcomas, and soft tissue sarcomas. In some embodiments, the tumor cells are tumor epithelial cells. In some embodiments, the tumor cells are tumor cells in epithelial-mesenchymal transition. In some embodiments, epithelial-mesenchymal transition is epithelial-mesenchymal transition state. In some embodiments, the tumor cells are tumor mesenchymal cells. In some embodiments, the tumor is an epithelial tumor. In some embodiments, the tumor is a mesenchymal tumor. In some embodiments, the tumor is an epithelial-mesenchymal tumor. As used herein, an epithelial-mesenchymal tumor is a tumor that has both epithelial and mesenchymal characteristics.

In some embodiments, the tumor cells and TME cells belong to a single primary or metastatic site. In some embodiments, a single primary or metastatic site is the same primary or metastatic site. In some embodiments, the primary or metastatic site is a site of the tumor. In some embodiments, the cancer is a solid cancer. In some embodiments, the cancer is a hematopoietic cancer. In some embodiments, the cancer is not a hematopoietic cancer. In some embodiments, the primary stie is selected from the group consisting of: gastrointestinal cancer, kidney cancer, liver cancer, breast cancer, bladder cancer, prostate cancer, ovary cancer, uterine cancer, head and neck cancer, thyroid cancer, glioma, neuroblastoma, melanoma, and lung cancer. In some embodiments, the cancer is selected from the group consisting of: gastrointestinal cancer, kidney cancer, liver cancer, breast cancer, bladder cancer, ovary cancer, uterine cancer, lung cancer, and any combination thereof. In some embodiments, the primary stie is selected from the group consisting of: gastrointestinal tract, kidney, liver, breast, bladder, prostate, ovary, uterus, head, neck, thyroid, brain, skin, and lung.

As used herein “cancer” or “pre-malignancy” are diseases associated with cell proliferation. Non-limiting types of cancer include carcinoma, sarcoma, lymphoma, leukemia, blastoma and germ cells tumors. In some embodiments, the cancer is solid cancer. In some embodiments, the cancer is a tumor. In some embodiments, the cancer is selected from hepato-biliary cancer, cervical cancer, urogenital cancer (e.g., urothelial cancer), testicular cancer, prostate cancer, thyroid cancer, ovarian cancer, nervous system cancer, ocular cancer, lung cancer, soft tissue cancer, bone cancer, pancreatic cancer, bladder cancer, skin cancer, intestinal cancer, hepatic cancer, rectal cancer, colorectal cancer, esophageal cancer, gastric cancer, gastroesophageal cancer, breast cancer (e.g., triple negative breast cancer), renal cancer (e.g., renal carcinoma), skin cancer, head and neck cancer, leukemia and lymphoma.

In one embodiment, carcinoma refers to tumors derived from epithelial cells including but not limited to breast cancer, prostate cancer, lung cancer, pancreas cancer, and colon cancer. In one embodiment, sarcoma refers to tumors derived from mesenchymal cells including but not limited to sarcoma botryoides, chondrosarcoma, ewings sarcoma, malignant hemangioendothelioma, malignant schwannoma, osteosarcoma and soft tissue sarcomas. In one embodiment, lymphoma refers to tumors derived from hematopoietic cells that leave the bone marrow and tend to mature in the lymph nodes including but not limited to hodgkin lymphoma, non-hodgkin lymphoma, multiple myeloma and immunoproliferative diseases. In one embodiment, leukemia refers to tumors derived from hematopoietic cells that leave the bone marrow and tend to mature in the blood including but not limited to acute lymphoblastic leukemia, chronic lymphocytic leukemia, acute myelogenous leukemia, chronic myelogenous leukemia, hairy cell leukemia, T-cell prolymphocytic leukemia, large granular lymphocytic leukemia and adult T-cell leukemia. In one embodiment, blastoma refers to tumors derived from immature precursor cells or embryonic tissue including but not limited to hepatoblastoma, medulloblastoma, nephroblastoma, neuroblastoma, pancreatoblastoma, pleuropulmonary blastoma, retinoblastoma and glioblastoma-multiforme. In one embodiment, germ cell tumors refers to tumors derived from germ cells including but not limited to germinomatous or seminomatous germ cell tumors (GGCT, SGCT) and nongerminomatous or nonseminomatous germ cell tumors (NGGCT, NSGCT). In one embodiment, germinomatous or seminomatous tumors include but not limited to germinoma, dysgerminoma and seminoma. In one embodiment, nongerminomatous or nonseminomatous tumors refers to pure and mixed germ cells tumors including but not limited to embryonal carcinoma, endodermal sinus tumor, choriocarcinoma, tearoom, polyembryoma, gonadoblastoma and teratocarcinoma.

In some embodiments, the tumor cells are primary cells from a subject and the stromal cells are primary cells from the same subject. In some embodiments, the tumor cells are primary cells from a subject and the endothelial cells are primary cells from the same subject. In some embodiments, the tumor cells are primary cells from a subject and the stromal cells and endothelial cells are primary cells from the same subject. In some embodiments, the tumor cells are primary cells from a subject and the stromal cells are primary cells from the same subject and the endothelial cells are from a cell line. In some embodiments, the cell line is an endothelial cell line. In some embodiments, the cell line is a tumor associated endothelial cell line. In some embodiments, the tumor cells are primary cells from a subject and the stromal cells are primary cells from the same subject and the endothelial cells are from a healthy subject. In some embodiments, the endothelial cells are human umbilical vein endothelial cells (HUVEC).

In some embodiments, the tumor avatar is a patient-personalized tumor avatar. In some embodiments, the patient is the subject. In some embodiments, the subject is the patient. In some embodiments, the tumor cells are from a subject and the TME cells are from the same subject. In some embodiments, the tumor cells are primary cells from a subject and the TME cells are from TMR cell lines. In some embodiments, the TME cells are present at a ratio found in a sample. In some embodiments, the sample is obtained from the patient. In some embodiments, the sample is derived from the patient. In some embodiments, the TECs and TASCs are present at a ratio found in the sample. In some embodiments, the tumor cells and TASCs are present at a ratio found in the sample. In some embodiments, the tumor cells and TECs are present at a ratio found in the sample. In some embodiments, the tumor cells, TECs and TASCs are present at a ratio found in the sample.

In some embodiments, the ratio is between 1:1 and 1:10. In some embodiments, the ratio is between 1:10 and 1:1. In some embodiments, the ratio is between 1:1 and 10:1. In some embodiments, the ratio is between 10:1 and 1:1. In some embodiments, the ratio is between 1:1 and 1:5. In some embodiments, the ratio is between 1:5 and 1:1. In some embodiments, the ratio is between 1:1 and 5:1. In some embodiments, the ratio is between 5:1 and 1:1. In some embodiments, the ratio is between 1:1 and 1:4. In some embodiments, the ratio is between 1:4 and 1:1. In some embodiments, the ratio is between 1:1 and 4:1. In some embodiments, the ratio is between 4:1 and 1:1. In some embodiments, between is ranging from.

In some embodiments, the ratio is between 1:1:1 and 1:1:10. In some embodiments, the ratio is between 1:1:10 and 1:1:1. In some embodiments, the ratio is between 1:1:1 and 10:1:1. In some embodiments, the ratio is between 10:1:1 and 1:1:1. In some embodiments, the ratio is between 1:1:1 and 1:10:1. In some embodiments, the ratio is between 1:10:1 and 1:1:1. In some embodiments, the ratio is between 1:1:1 and 1:1:5. In some embodiments, the ratio is between 1:1:5 and 1:1:1. In some embodiments, the ratio is between 1:1:1 and 5:1:1. In some embodiments, the ratio is between 5:1:1 and 1:1:1. In some embodiments, the ratio is between 1:1:1 and 1:5:1. In some embodiments, the ratio is between 1:5:1 and 1:1:1. In some embodiments, the ratio is between 1:1:1 and 1:1:4. In some embodiments, the ratio is between 1:1:4 and 1:1:1. In some embodiments, the ratio is between 1:1:1 and 4:1:1. In some embodiments, the ratio is between 4:1:1 and 1:1:1. In some embodiments, the ratio is between 1:1:1 and 1:4:1. In some embodiments, the ratio is between 1:4:1 and 1:1:1.

In some embodiments, the ratio is about 1:1. In some embodiments, the ratio is about 1:1:1. In some embodiments, the ratio is about 1:10. In some embodiments, the ratio is about 10:1. In some embodiments, the ratio is about 1:1:10. In some embodiments, the ratio is about 1:10:1. In some embodiments, the ratio is about 10:1:1. In some embodiments, the ratio is about 1:5. In some embodiments, the ratio is about 5:1. In some embodiments, the ratio is about 1:1:5. In some embodiments, the ratio is about 1:5:1. In some embodiments, the ratio is about 5:1:1. In some embodiments, the ratio is about 1:4. In some embodiments, the ratio is about 4:1. In some embodiments, the ratio is about 1:1:4. In some embodiments, the ratio is about 1:4:1. In some embodiments, the ratio is about 4:1:1.

In some embodiments, the tumor avatar further comprises a scaffold. In some embodiments, the scaffold is an artificial scaffold. In some embodiments, the scaffold is a manmade scaffold. In some embodiments, the scaffold is not found in a subject. In some embodiments, the scaffold is not found in vivo. In some embodiments, the scaffold comprises Matrigel. In some embodiments, the scaffold consists of Matrigel. In some embodiments, the scaffold comprises an extracellular matrix protein. In some embodiments, the scaffold comprises a glycoprotein. In some embodiments, the extracellular matrix protein is a glycoprotein. In some embodiments, the extracellular matrix protein is selected from vitronectin and fibronectin. In some embodiments, the extracellular matrix protein is fibronectin. In some embodiments, the scaffold comprises fibronectin. In some embodiments, the extracellular matrix protein is vitronectin. In some embodiments, the scaffold comprises vitronectin. In some embodiments, the scaffold is a 3D scaffold. In some embodiments, the scaffold is a gel. In some embodiments, the scaffold is not a coating. In some embodiments, the extracellular matrix protein is dissolved or embedded in the gel. In some embodiments, the gel is Matrigel. In some embodiments, the scaffold comprises any combination of Matrigel, fibronectin and vitronectin.

In some embodiments, the scaffold is a Matrigel dome. In some embodiments, the scaffold comprises a Matrigel dome. In some embodiments, the avatar is on low adherent plates. In some embodiments, the avatar is on adherent plates. In some embodiments, the avatar is on non-repellent plates. In some embodiments, the avatar is on low-repellent plates. In some embodiments, the culturing is on low adherent plates. In some embodiments, the culturing is on adherent plates. In some embodiments, the culturing is on non-repellent plates. In some embodiments, the culturing is on low-repellent plates. The various types of plates available for tissue culture are well known in the art and can be purchased from numerous vendors. In some embodiments, the Matrigel dome is on an adherent plate. In some embodiments, the Matrigel dome is on a non-repellent plate. The use of Matrigel domes for culturing organoids is well known in the art.

In some embodiments, the Matrigel is about 2% Matrigel. In some embodiments, the Matrigel is about 5% Matrigel. In some embodiments, the Matrigel is from 2%-5%. In some embodiments, the Matrigel dome comprises at least 20% Matrigel. In some embodiments, the Matrigel dome comprises more than 20% Matrigel. In some embodiments, the Matrigel dome comprises a Matrigel concentration of at least 20, 25, 30, 35, 40, 45, 50, 55, 60, 65, 70, 75, 80, 85, or 90% Matrigel. Each possibility represents a separate embodiment of the invention. In some embodiments, the Matrigel dome comprises a Matrigel concentration of at least 20%. In some embodiments, the Matrigel dome comprises a Matrigel concentration of at least 30%. In some embodiments, the Matrigel dome comprises a Matrigel concentration of at least 40%. In some embodiments, the Matrigel dome comprises a Matrigel concentration of at least 50%. In some embodiments, the Matrigel dome comprises a Matrigel concentration of 40-60%. In some embodiments, the Matrigel dome comprises a Matrigel concentration of 40-80%. In some embodiments, the Matrigel dome comprises a Matrigel concentration of about 40%. In some embodiments, the Matrigel dome comprises a Matrigel concentration of 30-50%. In some embodiments, the Matrigel dome comprises a Matrigel concentration of 35-45%. In some embodiments, the Matrigel dome comprises a Matrigel concentration of less than 60%.

In some embodiments, the extracellular matrix protein is present at about 5 ng/ul in the scaffold. In some embodiments, the extracellular matrix protein is present at about 10 ng/ul in the scaffold. In some embodiments, the extracellular matrix protein is present at a concentration from 5-10 ng/ul in the scaffold. In some embodiments, the extracellular matrix protein is present at a concentration from 1-10, 5-10, 1-20, 5-20, 10-20, 1-25, 5-25, 10-25, 1-30, 5-30, 10-30, 1-40, 5-40, 10-40, 1-50, 5-50 or 10-50 ng/ul in the scaffold. Each possibility represents a separate embodiment of the invention. In some embodiments, the extracellular matrix protein is present at a concentration from 1-50 ng/ul in the scaffold. In some embodiments, the scaffold comprises about 2% Matrigel and about 5 ng/ul extracellular matrix protein. In some embodiments, the scaffold comprises about 5% Matrigel and about 10 ng/ul extracellular matrix protein. In some embodiments, the extracellular matrix protein is selected from vitronectin and fibronectin.

In some embodiments, the scaffold comprises a concentration gradient. In some embodiments, the concentration gradient is a Matrigel concentration gradient. In some embodiments, the concentration gradient is an extracellular matrix protein concentration gradient. In some embodiments, the concentration gradient is a fibronectin concentration gradient. In some embodiments, the concentration gradient is a vitronectin concentration gradient. In some embodiments, the Matrigel gradient is from 1-100%. In some embodiments, the fibronectin gradient is from 0-50 ng/ul. In some embodiments, the fibronectin gradient is from 0.1-50 ng/ul. In some embodiments, the fibronectin gradient is from 1-50 ng/ul. In some embodiments, the vitronectin gradient is from 0-50 ng/ul. In some embodiments, the vitronectin gradient is from 0.1-50 ng/ul. In some embodiments, the vitronectin gradient is from 1-50 ng/ul.

By another aspect, there is provided a kit comprising a tumor avatar of the invention.

By another aspect, there is provided a kit comprising tumor cells and tumor microenvironment (TME) cells.

In some embodiments, the kit further comprises a scaffold. In some embodiments, the kit further comprises media. In some embodiments, the kit further comprises at least one media. In some embodiments, at least one is a plurality of media. In some embodiments, the media is liquid media. In some embodiments, the media is tissue culture media. In some embodiments, the media is suitable for culturing the tumor avatar. In some embodiments, the media is suitable for organization of the tumor avatar. In some embodiments, the media is suitable for maintenance of the tumor avatar. In some embodiments, the media is suitable for in vitro organization and maintenance of the tumor avatar. In some embodiments, the media is configured for culturing the tumor avatar.

In some embodiments, the media is suitable for culturing the tumor cells. In some embodiments, the media is suitable for culturing the tumor cells. In some embodiments, the media is suitable for culturing the TME cells. In some embodiments, the media is suitable for culturing the TECs. In some embodiments, the media is suitable for culturing the TASCs. In some embodiments, the media is suitable for culturing the adipose cells. In some embodiments, the media is suitable for culturing the immune cells. In some embodiments, the media is suitable for culturing the mesenchymal cells. In some embodiments, suitable for is configured for.

In some embodiments, the media comprises at least one component selected from the group consisting of: hepes, Antibiotic-Antimycotic, glutamax, non-essential amino acids, sodium pyruvate, R-Spondin, noggin, insulin growth factor 1 (IGF1), fibroblast growth factor 2 (FGF2), FGF10, epidermal growth factor (EGF), vascular endothelial growth factor (VEGF), Trombospondin (thrombospondin), ascorbic acid, Platelet-derived growth factor (PDGF), interleukin 6 (IL-6), IL-8, BMP4, BMP7, heparin, hydrocortisone, indomethacine, 3-Isobutyl-1-methylxanthine (IBMX), intralipids, bone morphogenetic protein 7 (BMP7), B27, A 83-01, N-Acetyl-L-cysteine, nicotinamide, and Y-27632. In some embodiments, the media comprises at least one component selected from the group consisting of: Hepes, ABAM, 2 GlutaMax supplement, R-spondin 1, Noggin, Wnt3a, epidermal growth factor (EGF), Gastrin, fibroblast growth factor 2 (FGF2), FGF10, gastrin, prostaglandin E2, A83-01, nicotinamide, and Vascular endothelial growth factor (VEGF), interleukin-6 (IL-6), IL-8, Bone Morphogenetic Protein 4 (BMP4) and BMP7. In some embodiments, at least one component is a plurality of components. In some embodiments, the media comprises at least one component selected from the group consisting of: hepes, abam, glutamax, non-essential amino acids, sodium pyruvate, R-Spondin, noggin, IGF1, FGF2, FGF10, EGF, VEGF, PDGF, IL-6, IL-8, heparin, indomethacine, IBMX, intralipids, BMP4, BMP7, B27, A 83-01, N-Acetyl-L-cysteine, A 83-01, nicotinamide, trombospondin, hydrocortisone, and Y-27632. In some embodiments, at least one is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. Each possibility represents a separate embodiment of the invention. In some embodiments, at least one is all of the components. In some embodiments, the proteins used as a component are human proteins. In some embodiments, the proteins used as a component are recombinant proteins.

In some embodiments, the media is Dulbecco's Modified Eagle Medium (DMEM). In some embodiments, the media is DMEM/F12. In some embodiments, the media is adherent cell media. In some embodiments, the media is tumor avatar media described herein below.

In some embodiments, the media comprises DMEM/F12. In some embodiments, the media comprises DMEM. In some embodiments, DMEM is DMEM/F12. In some embodiments, the media comprises Hepes. In some embodiments, the media comprises an antibiotic. In some embodiments, the media comprises Antibiotic-antimycotic (ABAM). In some embodiments, the media comprises GlutaMax. In some embodiments, the media comprises GlutaMax supplement. In some embodiments, the media comprises R-spondin. In some embodiments, R-spondin is R-spondin 1. In some embodiments, the media comprises Noggin. In some embodiments, the media comprises Wnt3a. In some embodiments, the media comprises epidermal growth factor (EGF). In some embodiments, the media comprises a fibroblast growth factor (FGF). In some embodiments, the FGF is FGF2. In some embodiments, the FGF is FGF10. In some embodiments, the media comprises FGF2. In some embodiments, the media comprises FGF10. In some embodiments, the media comprises both FGF2 and FGF10. In some embodiments, the media comprises gastrin. In some embodiments, the media comprises a prostaglandin. In some embodiments, the prostaglandin is prostaglandin E2. In some embodiments, the media comprises prostaglandin E2. In some embodiments, the media comprises A83-01. In some embodiments, the media comprises nicotinamide. In some embodiments, the media comprises SB202190. In some embodiments, the media comprises Y-27632. In some embodiments, the media comprises caspofungin. In some embodiments, the media comprises trombospondin (thrombospondin). In some embodiments, the media comprises hydrocortisone. In some embodiments, the media comprises VEGF. In some embodiments, the media comprises interleukin-6 (IL-6). In some embodiments, the media comprises IL-8. In some embodiments, the media comprises bone morphogenetic protein 4 (BMP4). In some embodiments, the media comprises BMP7. In some embodiments, the media comprises IGF-1. In some embodiments, the media does not comprise Insulin-like growth factor-1 (IGF-1). In some embodiments, the media is devoid of IGF-1. In some embodiments, the media does not comprise N-Acetyl-L-cysteine. In some embodiments, the media is devoid of N-Acetyl-L-cysteine. In some embodiments, the media comprises FGF-2, FGF-10, prostaglandin E2 and SB202190.

In some embodiments, the media comprises DMEM/F12, Hepes, ABAM, GlutaMax supplement, R-spondin 1, Noggin, Wnt3a, epidermal growth factor (EGF), fibroblast growth factor 2 (FGF2), FGF10, gastrin, prostaglandin E2, A83-01, nicotinamide, SB202190, Y-27632 and caspofungin. In some embodiments, the media is Medium 3. In some embodiments, the media comprises DMEM/F12, 10 mM Hepes, 1×ABAM, 2 mM GlutaMax supplement, 500 ng/ml R-spondin 1, 100 ng/ml Noggin, 100 ng/ml Wnt3a, 50 ng/ml EGF, 10 ng/ml FGF2, 10 ng/ml FGF10, 10 nM gastrin, 1 um prostaglandin E2, 0.5 μM A83-01, 4 mM nicotinamide, 5 μM SB202190, 10 μM Y-27632 and 0.5 ug/ml caspofungin. In some embodiments, Medium 3 comprises DMEM/F12, 10 mM Hepes, 1×ABAM, 2 mM GlutaMax supplement, 500 ng/ml R-spondin 1, 100 ng/ml Noggin, 100 ng/ml Wnt3a, 50 ng/ml EGF, 10 ng/ml FGF2, 10 ng/ml FGF10, 10 nM gastrin, 1 um prostaglandin E2, 0.5 μM A83-01, 4 mM nicotinamide, 5 μM SB202190, 10 μM Y-27632 and 0.5 ug/ml caspofungin.

In some embodiments, the media is mesenchymal stem cell growth medium. In some embodiments, the media is mesenchymal stem cell media (MSCM). Mesenchymal stem cell media is well known in the art and any such media may be used. Examples include, but are not limited to, commercially available media such as MSCM ScienCell, PluriSTEM Human ES/iPS Medium, MSC Growth Medium 2, and MSC NutriSTEM XF Medium. In some embodiments, the media is MSCM. In some embodiments, the media is fibroblast medium (FM). Fibroblast media is well known in the art and any such media may be used. Examples include, but are not limited to, commercially available media such as Fibroblast Medium-2 (FM-2) ScienCell, Fiboblast Basal Medium, FibroLife S2 Fibroblast Medium, and FGM-2. In some embodiments, the media is FM-2. In some embodiments, the media is endothelial cell growth medium. In some embodiments, endothelial cell growth media is endothelial cell media (ECM). Endothelial cell media is well known in the art and any such media may be used. Examples include, but are not limited to, commercially available media such as ECM ScienCell, MSCM 7501-scl ScienCell Endothelial Cell Medium, 1001-SCL ScienCell, 2331-SCL ScienCell, EGM-2 Endothelial Cell Medium and EndoGo XF Medium. In some embodiments, the media is ECM. In some embodiments, the media is adipocyte media. Adipocyte cell media is well known in the art and any such media may be used. Examples include, but are not limited to, commercially available media such as Adipocyte Nutrition Medium and Human Adipocyte and Preadipocyte Medium. In some embodiments, the media is immune cell media. In some embodiments, the media is lymphocyte media. In some embodiments, the media is suspension cell media. Immune cell media is well known in the art and any such media may be used. Examples include, but are not limited to, commercially available media such as RPMI, RPMI1640, ExCellerate immune cell Medium, ImmunoCult immune cell Medium, Immune cell PRIME-XV Medium, and LGM-3 lymphocyte growth medium. Media for a variety of cell types are commercially available from companies such as Biological Industries, Sigma-Aldrich, ACROBiosystems, Lonza, RNDsystems, Thermo Fisher Scientific and many others.

In some embodiments, the media is selected from DMEM/F12, ECM, FM and MSCM. In some embodiments, FM is FM-2. In some embodiments, the media is a combination of media. In some embodiments, the combination is ECM:FM:MSCM. In some embodiments, the combination is DMEM/F12:ECM:FM:MSCM. In some embodiments, the ECM:FM:MSCM is present in a ratio of 1:1:1. In some embodiments, the ratio is by volume. In some embodiments, the DMEM/F12:ECM:FM:MSCM is present in a ratio of 1:1:1:1. In some embodiments, the DMEM/F12:ECM:FM:MSCM is present in a ratio of 10:1:1:1. In some embodiments, the DMEM/F12:ECM:FM:MSCM is present in a ratio of 1-10:1:1:1.

In some embodiments, the media is avatar media as described herein. In some embodiments, the media is the media provided in Table 2. In some embodiments, the media is the media provided in Table 3. In some embodiments, the avatar comprises immune cells and the media further comprises at least one of: IL-2, beta-mercaptoethanol, IL-4, IL-13 and Macrophage colony-stimulating factor (M-CSF). In some embodiments, the avatar comprises immune cells and the media further comprises: IL-2, beta-mercaptoethanol, IL-4, IL-13 and Macrophage colony-stimulating factor (M-CSF).

In some embodiments, the tumor is derived from a gastrointestinal tumor. In some embodiments, the tumor cells are derived from a gastrointestinal cancer. In some embodiments, the media further comprises at least one component selected from the group consisting of: Wnt family member 3A (WNT3A), gastrin, hepatocyte growth factor (HGF) and prostaglandin E2. In some embodiments, the media further comprises at least one component selected from the group consisting of: WNT3A, gastrin and prostaglandin E2.

In some embodiments, the tumor is derived from a kidney tumor. In some embodiments, the tumor cells are derived from a kidney cancer. In some embodiments, the media further comprises at least one component selected from the group consisting of: HGF, epinephrine, hydrocortisone and FGF8. In some embodiments, the media further comprises at least one component selected from the group consisting of: hydrocortisone and epinephrine.

In some embodiments, the tumor is derived from a liver tumor. In some embodiments, the tumor cells are derived from a liver cancer. In some embodiments, the media further comprises HGF.

In some embodiments, the tumor is derived from a lung tumor. In some embodiments, the tumor cells are derived from a lung cancer. In some embodiments, the media further comprises FGF7.

In some embodiments, the tumor is derived from a breast tumor. In some embodiments, the tumor cells are derived from a breast cancer. In some embodiments, the tumor is derived from an ovary or uterine tumor. In some embodiments, the tumor cells are derived from an ovary or uterine cancer. In some embodiments, the media further comprises heregulin. In some embodiments, the media further comprises at least one component selected from the group consisting of: heregulin, and B-Estradiol. In some embodiments, the media further comprises at least one component selected from the group consisting of: heregulin, hydrocortisone and β-Estradiol.

In some embodiments, the tumor is derived from a bladder tumor. In some embodiments, the tumor cells are derived from a bladder cancer. In some embodiments, the media further comprises FGF7, heregulin or both.

In some embodiments, the tumor avatar comprises adipocytes. In some embodiments, the media further comprises indomethacin, 3-Isobutyl-1-methylxanthine (IBMX) or both. In some embodiments, the tumor avatar comprises immune cells. In some embodiments, the media further comprises at least one component selected from the group consisting of: anti-CD3 antibody, anti-CD28 antibody, glutamine, human AB serum, HEPES buffer, β-mercaptoethanol and IL-2. In some embodiments, the media further comprises at least one component selected from the group consisting of: anti-CD3 antibody, anti-CD28 antibody, β-mercaptoethanol and IL-2.

In some embodiments, the method further comprises biobanking produced cells. In some embodiments, the method further comprises freezing produced cells. In some embodiments, the method further comprises thawing frozen cells. In some embodiments, thawed cells are used in the culturing of step (b).

By another aspect, there is provided a method of producing a tumor avatar, the method comprising:
a. providing tumor cells and TME cells; and
b. culturing the tumor cells and TME cells with an artificial scaffold in media suitable for organization and maintenance of a tumor avatar;
   thereby producing a tumor avatar.

In some embodiments, the tumor avatar is a tumor avatar of the invention. In some embodiments, the method is a method of producing a tumor avatar of the invention.

In some embodiments, the method further comprises isolating the tumor cells. In some embodiments, the method further comprises isolating the TME cells. In some embodiments, the isolating is before step (a). In some embodiments, isolating is acquiring. In some embodiments, isolating is providing. In some embodiments, isolating is receiving. In some embodiments, the cells are isolated from a sample. In some embodiments, the sample comprises tumor tissue. In some embodiments, the sample comprises tumor surrounding tissue. In some embodiments, the sample comprises cancer cells and/or TME cells. In some embodiments, the sample is a biopsy. In some embodiments, stromal cells are isolated from a sample. In some embodiments, the TME cells comprise TECs and CAFs.

In some embodiments, providing TME cells comprises receiving isolated stromal cell from a tumor or surrounding tissue thereof. In some embodiments, stromal cells do not express at least one epithelial cell marker. In some embodiments, stromal cells are devoid of expression of at least one epithelial cell marker. In some embodiments, the epithelial cell marker is selected from EPCAM and E-cadherin. In some embodiments, providing TME cells comprises receiving isolated tumor associated stromal cells. In some embodiments, the received stromal cells are from a tumor or surrounding tissue thereof in a subject. In some embodiments, providing TME cells further comprises culturing a first portion of the received isolated stromal cells in ECM. In some embodiments, providing TME cells further comprises culturing a second portion of the received isolated stromal cells in fibroblast media (FM).

In some embodiments, the culturing in ECM is for a sufficient time to enrich for iCAFs. In some embodiments, the culturing in ECM is for a sufficient time to produce increased expression of an iCAF marker. In some embodiments, the culturing a first portion is for 1-3 weeks. In some embodiments, the culturing in FM is for a sufficient time to enrich for eCAFs. In some embodiments, the culturing in FM is for a sufficient time to produce increased expression of an eCAF marker. In some embodiments, the culturing a second portion is for 1-3 weeks. In some embodiments, step (b) comprises culturing the tumor cells, the first portion and the second portion together. In some embodiments, culturing the first portion enriches endothelial cells. In some embodiments, the enriched endothelial cells population is cultured with the tumor cells in step (b). In some embodiments, culturing the second portion enriches fibroblasts. In some embodiments, the enriched fibroblast population is cultured with the tumor cells in step (b). In some embodiments, the enriched fibroblast and the enriched endothelial cells are cultured with the tumor cells in step (b).

In some embodiments, an enriched endothelial cell population comprises increased expression of at least one endothelial cell marker as compared to the isolated stromal cells before culture. In some embodiments, an enriched endothelial cell population comprises increased expression of at least one endothelial cell marker as compared to cells cultured in FM. In some embodiments, the at least one endothelial cell marker is selected from CD146 (MCAM) and VE-cadherin.

In some embodiments, providing TME cells comprises culturing a third portion of the received isolated stromal cells in mesenchymal stem cell medium MSCM. In some embodiments, providing TME cells comprises culturing a fourth portion of the received isolated stromal cells in a combination media. In some embodiments, the combination media is MSCM:ECM:FM. In some embodiments, the MSCM:ECM:FM is present in a ratio of 1:1:1. In some embodiments, the ratio is by volume. In some embodiments, the culturing a third portion is for 1-3 weeks. In some embodiments, the culturing a fourth portion is for 1-3 weeks. In some embodiments, a sufficient time is 1-3 weeks.

In some embodiments, the enriched fibroblast and HUVEC cells are cultured with the tumor cells in step (b). In some embodiments, providing TASCs comprises receiving isolated stromal cells from a tumor or a surrounding tissue thereof and culturing the received isolated stromal cells in FM and the providing TECs comprises providing cells from a TEC cell line.

In some embodiments, at least 80, 85, 90, 92, 95, 97 or 99% of the stromal cells express vimentin. Each possibility represents a separate embodiment of the invention. In some embodiments, at least 95% of the stromal cells express vimentin. In some embodiments, at least 80, 85, 90, 92, 95, 97 or 99% of the TME cells express vimentin. Each possibility represents a separate embodiment of the invention. In some embodiments, at least 95% of the TME cells express vimentin. In some embodiments, at least 80, 85, 90, 92, 95, 97 or 99% of the stromal cells express at least one MSC marker selected from CD105, CD90 and CD73. Each possibility represents a separate embodiment of the invention. In some embodiments, at least 95% of the stromal cells express at least one MSC marker selected from CD105, CD90 and CD73. In some embodiments, at least 80, 85, 90, 92, 95, 97 or 99% of the TME cells express at least one MSC marker selected from CD105, CD90 and CD73. Each possibility represents a separate embodiment of the invention. In some embodiments, at least 95% of the TME cells express at least one MSC marker selected from CD105, CD90 and CD73. In some embodiments, at least one MSC marker is at least two MSC markers. In some embodiments, at least one MSC marker is all three of CD105, CD90 and CD73.

In some embodiments, the received isolated stomal cells are cultured in mesenchymal stem cell medium (MSCM). In some embodiments, the culturing in MSCM is before culturing the first portion and/or the second portion. In some embodiments, the received isolated stomal cells are cultured in MSCM and then in FM. In some embodiments, the received isolated stomal cells are cultured in MSCM and then in ECM. In some embodiments, the received isolated stomal cells are cultured in MSCM and then a first portion of the cells cultured in MSCM are cultured in ECM a second portion of the cells cultured in MSCM are cultured in FM. In some embodiments, the culturing in MSCM is for 2-3 weeks. In some embodiments, the culturing in MSCM is for at least 3 weeks. In some embodiments, the culturing in MSCM is for about 3 weeks. In some embodiments, the culturing in MSCM is for 2-4 weeks.

In some embodiments, providing tumor cells comprises receiving tumor cells. In some embodiments, the received tumor cells are cultured into organoids. In some embodiments, organoids are spheroids. In some embodiments, the organoid cells are cultured with the TME cells in step (b). In some embodiments, the organoids are cultured with the TME cells in step (b). In some embodiments, culturing into organoids comprises culturing in media. In some embodiments, culturing into organoids comprises culturing in media comprising DMEM/F12, Hepes, ABAM, 2 GlutaMax supplement, R-spondin 1, Noggin, Wnt3a, epidermal growth factor (EGF), fibroblast growth factor 2 (FGF2), FGF10, gastrin, prostaglandin E2, A83-01, nicotinamide, SB202190, Y-27632 and caspofungin to produce organoids. In some embodiments, the media is Medium 3. In some embodiments, the media comprises DMEM/F12, 10 mM Hepes, 1×ABAM, 2 mM GlutaMax supplement, 500 ng/ml R-spondin 1, 100 ng/ml Noggin, 100 ng/ml Wnt3a, 50 ng/ml EGF, 10 ng/ml FGF2, 10 ng/ml FGF10, 10 nM gastrin, 1 um prostaglandin E2, 0.5 μM A83-01, 4 mM nicotinamide, 5 μM SB202190, 10 μM Y-27632 and 0.5 ug/ml caspofungin. In some embodiments, Medium 3 comprises DMEM/F12, 10 mM Hepes, 1×ABAM, 2 mM GlutaMax supplement, 500 ng/ml R-spondin 1, 100 ng/ml Noggin, 100 ng/ml Wnt3a, 50 ng/ml EGF, 10 ng/ml FGF2, 10 ng/ml FGF10, 10 nM gastrin, 1 um prostaglandin E2, 0.5 μM A83-01, 4 mM nicotinamide, 5 μM SB202190, 10 μM Y-27632 and 0.5 ug/ml caspofungin.

In some embodiments, organization is organization such as is described hereinbelow. In some embodiments, organization is organization into a tumor avatar. In some embodiments, maintenance comprises retaining about the same ratio of cells as were initially seeded in the scaffold. In some embodiments, culturing the cells comprises seeding the cells in the scaffold. In some embodiments, maintenance comprises supporting cell survival. In some embodiments, survival is survival of at least 50, 55, 60, 65, 70, 75, 80, 85, 90, 92, 95, 97, 98, 99 or 100% of the cells. Each possibility represents a separate embodiment of the invention.

In some embodiments, the culturing is for at least 1 day. In some embodiments, the culturing is for at least 1, 3, 5, 7, 10, 14, 21, 28, 30, 35, 42, 49, 56, 60 or 63 days. Each possibility represents a separate embodiment of the invention. In some embodiments, the culturing is for at most two months. In some embodiments, the culturing is for at most 1 week, 2 weeks, 3 weeks, 4 weeks, 1 month, 5 weeks, 6 weeks, 7 weeks, 8 weeks or 2 months. Each possibility represents a separate embodiment of the invention. In some embodiments, the culturing is for between 1 day and 2 months.

In some embodiments, the culturing is with shaking. In some embodiments, the culturing is in a rotary shaker. In some embodiments, the culturing is in hypoxia. In some embodiments, the culturing is in normoxia. In some embodiments, the culturing is at about 2% oxygen (O₂). In some embodiments, the culturing is at about 4% oxygen. In some embodiments, the culturing is at about 20% oxygen. In some embodiments, the culturing is at about 2% to about 20% oxygen.

In some embodiments, the tumor cells are a preserved cell line. In some embodiments, the TME cells are a preserved cell line. In some embodiments, the method further comprises obtaining the persevered cell line. In some embodiments, the obtaining is before step (b). In some embodiments, the obtaining comprises culturing the tumor cells, TME cells or both in specialized media designed to enrich the tumor of the tumor cells, TME cells or both, thereby obtaining the preserved cell line. In some embodiments, the obtaining comprises culturing the tumor cells, TME cells or both in specialized scaffold designed to enrich the tumor of the tumor cells, TME cells or both, thereby obtaining the preserved cell line.

In some embodiments, the tumor cells comprise epithelial cells and the specialized media comprises epithelial cell growth media. In some embodiments, the TME cells comprise TECs and the specialized media comprises TEC growth media. In some embodiments, the TME cells comprise TECs and the specialized media comprises endothelial cell growth media. In some embodiments, TEC growth media is endothelial cell growth media. In some embodiments, endothelial cell growth media comprises MSC media, fibroblast media and endothelial media. In some embodiments, the TME cells comprise TASCs and the specialized media comprises TASC growth media. In some embodiments, the TME cells comprise TASCs and the specialized media comprises stromal cell growth media. In some embodiments, TASC growth media is stromal cell growth media. In some embodiments, stromal cell growth media comprises mesenchymal stem cell (MSC) growth media. In some embodiments, stromal cell growth media comprises fibroblast growth media. In some embodiments, stromal cell growth media comprises endothelial cell media. In some embodiments, stromal cell growth media comprises MSC media, fibroblast media and endothelial media. In some embodiments, media is growth media. In some embodiments, the ratio between MSC media, fibroblast media and endothelial media is about 1:1:1. In some embodiments, the ratio between MSC media, fibroblast media and endothelial media is about 1:1:4. In some embodiments, the ratio is by volume. In some embodiments, the ratio is by weight. In some embodiments, the TME cells comprise TAAs and the specialized media comprises adipocyte cell growth media. In some embodiments, the TME cells comprise TAAs and the specialized media comprises TAA growth media. In some embodiments, TAA growth media is adipocyte growth media. In some embodiments, the TME cells comprise immune cells and the specialized media comprises immune cell growth media. In some embodiments, the TME cells comprise TILs and the specialized media comprises TIL growth media. In some embodiments, TIL growth media is immune cell growth media. In some embodiments, TIL growth media is lymphocyte cell growth media.

In some embodiments, the method further comprises receiving a tumor sample. In some embodiments, the sample is from a subject. In some embodiments, the subject is a mammal. In some embodiments, the mammal is a human. In some embodiments, the subject is in need of a tumor avatar. In some embodiments, the subject suffers from cancer. In some embodiments, the subject is in need of the performance of a method of the invention.

In some embodiments, the sample comprises tumor cells and TME cells. In some embodiments, the TME cells are TECs and TASCs. In some embodiments, the method further comprises determining the number of tumor cells. In some embodiments, the method further comprises determining the number of TME cells. In some embodiments, the method further comprises determining the number of each type of TME cell. In some embodiments, the method further comprises determining the number of TECs. In some embodiments, the method further comprises determining the number of TASCs. In some embodiments, the method further comprises determining the ratio between tumor cells and TME cells. In some embodiments, the method further comprises determining the ratio between different types of TME cells. In some embodiments, the types of TME cells are selected from TACS, TECs, TAAs and immune cells. In some embodiments, the method further comprises determining the ratio between TECs and TASCs. In some embodiments, the method further comprises determining the ratio between tumor cells, TECs and TASCs. In some embodiments, the number is the number in the sample. In some embodiments, the ratio is the ratio in the sample. In some embodiments, the number and/or ratio in the sample is indicative of the number and/or ratio in the subject. In some embodiments, step (b) further comprises culturing immune cells. In some embodiments, the immune cells are tumor associated. In some embodiments, the immune cells are PBLs.

In some embodiments, the culturing comprises culturing according to the determined ratio. In some embodiments, the culturing comprises culturing the cells at about the determined ratio. In some embodiments, the culturing ratio is up to +1, 2, 3, 5, 7, 10, 12, 15, 17, 20, 22, 25, 27, or 30% of the determined ratio. Each possibility represents a separate embodiment of the invention. In some embodiments, the culturing ratio is up to +20% of the determined ratio.

By another aspect, there is provided a tumor avatar produced by a method of the invention.

By another aspect, there is provided a method for screening a drug, the method comprising:
a. culturing a tumor avatar in media;
b. contacting the tumor avatar with the drug; and
c. measuring an effect of the drug in the tumor avatar;
   thereby screening a drug.

In some embodiments, the tumor avatar is a tumor avatar of the invention. In some embodiments, the method further comprises producing the tumor avatar. In some embodiments, the producing is by a method of the invention. In some embodiments, the producing is before step (a). In some embodiments, the culturing is in culture media. In some embodiments, the contacting comprises contacting the media with the drug. In some embodiments, the contacting is adding the drug to the media. In some embodiments, the contacting comprises culturing the tumor avatar in the presence of the drug. In some embodiments, the culturing in the presence of the drug is for a time sufficient for an effect to be produced.

In some embodiments, the drug is an anticancer drug. In some embodiments, the drug is a drug being considered for administration to the subject. In some embodiments, the drug is a plurality of drugs. In some embodiments, each drug of the plurality of drugs is separately contacted to a tumor avatar. In some embodiments, the drug is a combination of drugs. In some embodiments, the effect is an anticancer effect. In some embodiments, the anticancer effect is reduced cell viability. In some embodiments, viability is viability of the cancer cells. In some embodiments, viability is viability of the tumor avatar. In some embodiments, the anticancer effect is reduced proliferation. In some embodiments, proliferation is proliferation rate. In some embodiments, the anticancer effect is shrinking of the tumor avatar. In some embodiments, the anticancer effect is cell death.

In some embodiments, the presence of the effect above a predetermined threshold indicates the drug is suitable for being a drug. In some embodiments, a method of screening is a method of determining suitability of the drug. In some embodiments, suitability is suitability as an anticancer drug. In some embodiments, suitability is suitability to treat cancer. In some embodiments, suitability is suitability to treat a cancer upon which the tumor avatar is based. In some embodiments, suitability is suitability to treat a cancer type upon which the tumor avatar is based. In some embodiments, the method is a method of personalized drug screening. In some embodiments, the method is a method of personalized drug selection. In some embodiments, suitability is suitability to treat a subject that provided the tumor cells for the tumor avatar. In some embodiments, suitability is suitability to treat a subject with the same type of cancer as the type of cancer in the tumor avatar. In some embodiments, type is the same histological type. In some embodiments, type is the same primary location in the body of the tumor cells. In some embodiments, the primary location is the origin of the tumor cells. In some embodiments, suitability is suitability to treat a subject with the same ratio of tumor cells to TME cells as the tumor avatar. In some embodiments, the same is about the same.

In some embodiments, the predetermined threshold is the presence of the effect in the tumor avatar culture without a drug. In some embodiments, the predetermined threshold is the presence of the effect in a tumor avatar culture contacted with a control drug. In some embodiments, the control drug is a placebo. In some embodiments, the control drug is not an anticancer drug. In some embodiments, the method further comprises administering to a subject a drug determined to be suitable.

In some embodiments, the method comprises before step (a) selecting the drug. In some embodiments, the drug is selected by a method comprising:
a. receiving transcriptomic data from the tumor cells and the TME cells;
b. identifying at least one gene differentially expressed in the tumor cells as compared to the TME cells; and
c. selecting a drug that targets the at least one gene or its protein product.

In some embodiments, the gene is upregulated in the TME cells and the drug inhibits, blocks or degrades the gene or its protein product. In some embodiments, the gene is down regulated in the TME cells and the drug increases, enhances or blocks degradation of the gene or its protein product.

Anticancer drugs are selected based on patient clinical state or/and the fold change in RNA expression between tumor and stroma cells. Targeted drugs for a specific patient are prioritized based on transcriptomic data from tumor-derived organoids and stromal cells. Drugs related to the genes differentially expressed in organoids as compared to stromal cells were identified using available databases (such as PanDrugs) or in the patient's cells themselves. Other secondary target genes of the selected drugs, that also have low constant inhibition or IC50 value in the presence of the specific drug, are considered. Next, drugs with the highest number of related genes up regulated in organoids (both with increased expression and low constant inhibition or IC50 value for the specific drug) are tested used on the organoid. Genes with high RNA level evidence score were considered. This score was ranked based on the following factors: published evidence regarding the relationship of these genes with the specific cancer type tested, existence of drugs targeting the specific gene (clinical use, clinical trial phase) and the role of the gene in cancer oncogenesis, cell proliferation, cell death, prognosis, and progression.

In some embodiments, the method further comprises producing a Strengths, Weaknesses, Opportunities, and Threats (SWOT) analysis for the drug. In some embodiments, a plurality of drugs is tested. In some embodiments, the tested drugs are categorized from best potential to worst potential. In some embodiments, the best drug is selected. In some embodiments, the best drug is administered to the subject. In some embodiments, the method further comprises administering a selected drug to the subject. In some embodiment, the selected drug is the best drug. In some embodiments, the categorizing is by SWOT analysis. In some embodiments, the SWOT analysis is for the drug as an anticancer therapeutic for the subject. In some embodiments, the SWOT analysis is for the drug as an anticancer therapeutic against the cancer of the avatar. A drug categorization report using a SWOT analysis is made to describe the drug's potential effectiveness for a specific avatar or patient. Strengths included FDA-approved drugs with high RNA expression, fold change, and level of evidence scores, as well as high cytotoxic activity in the co-culture model. Opportunities involve repurposing drugs and targeting drugs in clinical trials, with favorable scores in RNA expression, fold change, and level of evidence, and demonstrated cytotoxicity in both co-culture and organoid models. Weaknesses comprise drugs causing up to 30% cell death in the co-culture but more than 50% cell death in organoids. Threats consist of drugs with low RNA scores or no cytotoxicity in the ex-vivo assay.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

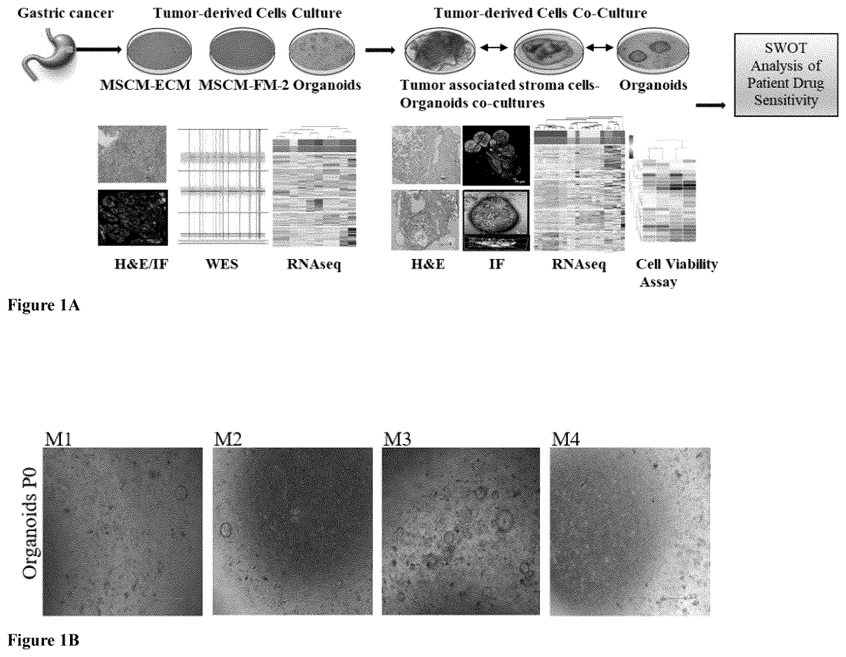

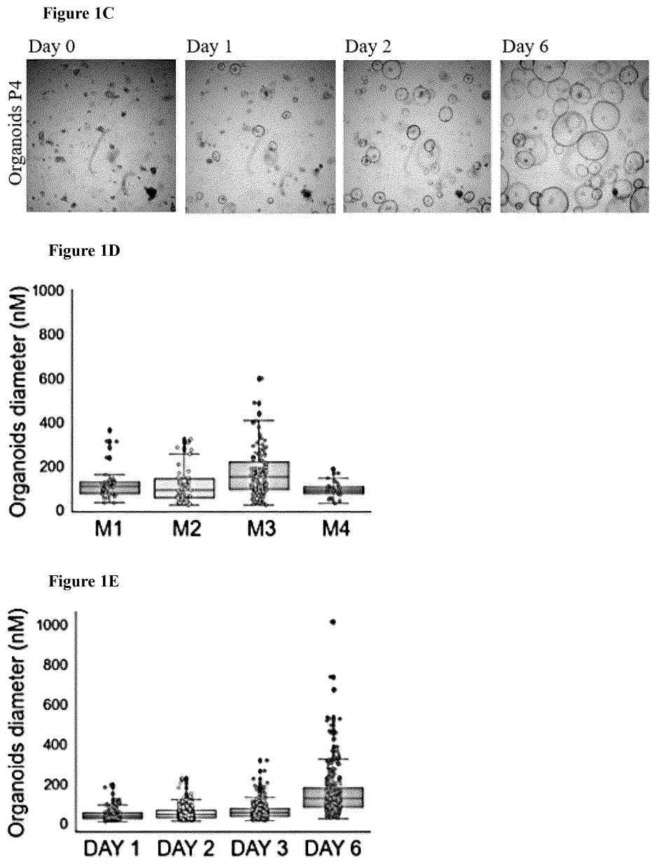

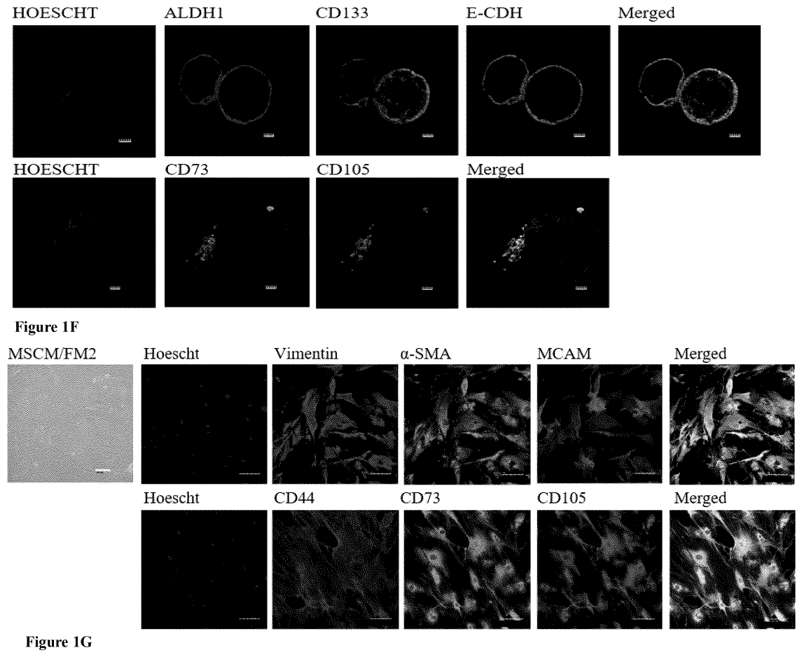

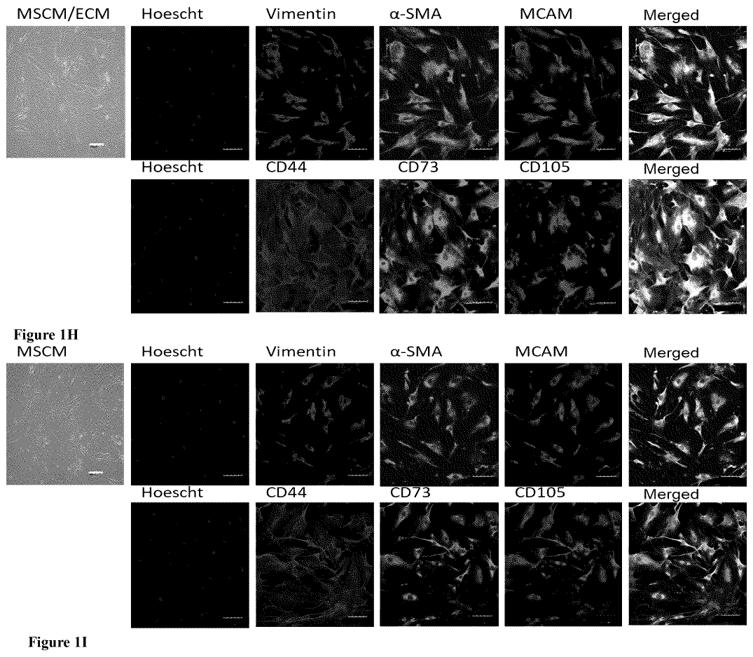

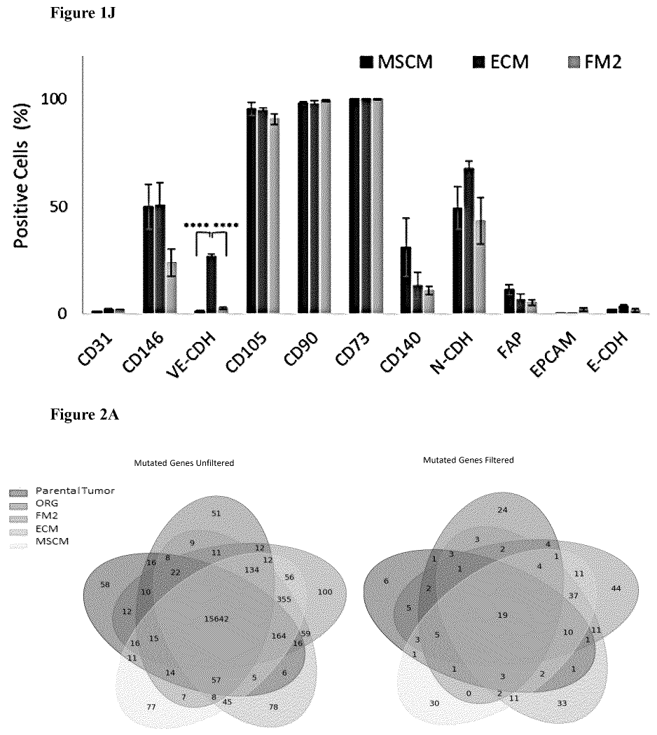

Study design: The inventors aimed to develop a three-dimensional (3D) in vitro model, termed “mini-tumor”, or “tumor avatar”, that is composed of tumor cells and tumor microenvironmental (TME) cells. The tumor cells are either primary patient-derived tumor cells or a tumor cell line, obtained after enrichment of the cells in a specialized growth medium. TME cells are either cells derived from the same tumor sample as the tumor cells, cells present in the tissue surrounding the tumor, or alternatively, cells derived from a pool of TME cells obtained from cancer patients with the same tumor characteristics, histologic types or/and molecular profile, as the tumor cells. Similar to the tumor cells, TME cells are either primary cells or TME cell lines. FIG. 1A offers a comprehensive overview of our experimental design and methodology.

Organoids and stroma cells isolation and culture: Human gastric cancer along with surrounding normal tissue were obtained from a consenting patient undergoing surgery at Ichilov Hospital, with approval from the Institutional Review Board. The samples were transported in cold hypothermosol medium and processed within 24 hours post-surgery. Upon acquisition, the tumor tissue was divided into three equal parts for various analyses. One third was snap-frozen for subsequent DNA isolation, another third was fixed in 4% paraformaldehyde for histological examination, and the remaining portion was prepared for cell culture. The tissue underwent digestion using Collagenase I (1 mg/ml), Liberase, and DNAse (10 ug/ml), followed by separation into organoids and stroma cell cultures. Organoids were cultured as previously described hereinbelow, with medium replacement twice weekly and passaging via mechanical and enzymatic dissociation using TrypLE enzyme. Stroma cells were cultured in mesenchymal stem cell medium (MSCM) on pre-coated Matrigel (20%) and fibronectin (2 μg/cm2) 4-well dish plates. Upon reaching 80% confluency the cells were subculturing using accutase to 6 well plate and then to 25 cm flask. From passage 3 onwards, cells were sub-cultured in 25 cm flask and then in 75 cm flasks using MSCM, endothelial cell medium (ECM), or fibroblast medium (FM-2) for subsequent passages. Bright field images were obtained using Operetta High Content Screening System or Leica microscope.

Tumor tissues processing: Tumor tissues or biopsy samples were minced into 50-500 μm slices. The slices were mechanically disrupted or enzymatically treated until the extracellular matrix was partially digested. Then, the enzymatic reaction was neutralized by the addition of a medium with 10% serum. Samples were centrifuged and the slices were embedded in a biological or synthetic scaffold and transferred to well dishes. The plates were incubated at 37° C. under normal or hypoxic conditions in a culture medium. Samples were incubated in 5% CO₂ and 20% or 2% O₂ at 37° C. in a CO₂ incubator. To generate a fluid flow, the culture dishes were placed on a rotatory shaker at 15-50 rpm throughout the culture period. Normal tissues from healthy donors were processed in a similar manner and served as control.

Alternatively, tumor tissues or biopsy samples were partially dissociated to clusters of cells by mechanical or enzymatic dissociation, and cultured in a specific cell type enrichment medium, in order to obtain a population or subpopulation cell line.

Tumor cell and TME cell cultures: For establishment of a tumor epithelial cell culture, tumor epithelial cells were grown in 2D (spheroids) or 3D (organoids) cultures according to established protocols.

To obtain a tumor niche cell culture, dissociated TME cells were plated in a plate covered with a scaffold or a combination of biological and/or synthetic scaffolds (e.g., Matrigel, collagen, gelatin, agar, fibronectin, vitronectin, hyaluronic acid, laminin, chitosan, alginate, methylcellulose, poly-L-lysine and/or poly-D-lysine alone or in combination such as Matrigel and fibronectin, gelatin and alginate). TME cells were grown in parallel in: i) a commercial mesenchymal stem cells growth medium and ii) in a medium composed of: mesenchymal stem cell growth medium, fibroblast growth medium, and endothelial cell growth medium at an equal (v/v) ratio (1:1:1). Cells were grown as 2D cultures or 3D cultures with the addition of microcarriers (such as corning or solohill microcarriers) when necessary to accelerate cell growth.

To obtain cancer-associated fibroblasts (CAFs), dissociated TME cells were plated in a plate covered with a specific scaffold or a combination of biological and/or synthetic scaffolds (e.g., Matrigel, collagen, gelatin, agar, fibronectin, vitronectin, hyaluronic acid, laminin, chitosan, alginate, methylcellulose, poly-L-lysine and/or poly-D-lysine alone or in combination such as Matrigel and fibronectin, gelatin and alginate). The TME cells were grown in a commercial fibroblast growth medium as 2D culture or 3D culture with addition of microcarriers (such as corning or solohill microcarriers) when necessary to accelerate cells growth.

To obtain a tumor endothelial cell (TECs) culture, dissociated TME cells were plated in a plate covered with a specific scaffold or a combination of biological and/or synthetic scaffolds (e.g., Matrigel, fibronectin, vitronectin, collagen, gelatin, laminin, hyaluronic acid, chitosan, PEG, and/or agar). The TME cells were grown in a commercial endothelial cell medium as 2D cultures or 3D cultures with addition of microcarriers (such as corning or solohill microcarriers) when necessary to accelerate cells growth.

Alternatively, TECs were isolated from niche tumor cells and CAF cultures, via magnetic separation by using magnetic anti-CD31 conjugated beads. CD31 positive cells were grown in endothelial cell medium, or in a medium composed of mesenchymal stem cell growth medium, fibroblast growth medium, and endothelial cell growth medium at a v/v ratio of 1:1:4, respectively. CD31 negative cells were grown in a medium composed of mesenchymal stem cell growth medium and fibroblast growth medium (1:3) and cells named TASCs.

To obtain immune cell cultures, tumor tissue was enzymatically dissociated to a single-cell suspension. After pulse centrifugation, the supernatant (immune cells and single cancer cells) and cell pellet (cancer cell aggregates) were separated and collected. Then, CD14 MicroBeads were used to enrich CD14 positive cells from the supernatant. Macrophages were obtained by allowing the CD14 positive cells to adhere to a fibronectin coated glass bottom plate for 1 h. Non-adherent cells were collected and cultured together with the CD14 negative cell fraction. CD14+adherent fraction was resuspended in RPMI supplemented with 10% heat-inactivated FBS and 1% P/S. Non-adherent cells and the CD14 negative cell fraction from the pellet contain lymphocytes. These cells were seeded in 24 well plates and cultured in a complete medium such as LGM-3 lymphocyte growth medium or RPMI1640 supplemented with glutamine (2 mM), 10% AB serum, 1% Penicillin-Streptomycin, HEPES buffer (25 mM), β-mercaptoethanol (55 μM) and rhIL-2 (6,000 IU/mL). Short-term cultured immune cells were cryopreserved or used for further expansion. For specific assays, B cells were stimulated with rhIL-4 (10 ng/ml), Pokeweed mitogen 10 (ug/mL)), 12-O-tetradecanoylphorbol 13-acetate (910 ng/ml) and calcium ionophore (1 μg/mL). For T cell activation, Dynabeads Human T activator CD3/CD28 were added at a 1:1 bead:cell ratio per manufacturer's instructions. In parallel, mononuclear cells from autologous or allogenic peripheral blood were obtained by Ficoll-Hypaque, fractionated in CD14 positive and CD14 negative populations and cultivated as described.

Cryopreservation of TME cell lines: The dissociated and cultured patient derived tumor cells and TME cells were cryopreserved in a freezing medium or by vitrification and stored in liquid nitrogen. TME cells were cryopreserved as a mix of TME cells, as a TASC cell line and a TEC cell line, or as a cell subpopulation cell line, such as tumor associated mesenchymal stem cells (TA-MSCs), CAFs, TAAs, peripheral blood polymorphonuclear cells, peripheral blood mononuclear cells or/and tumor infiltrated immune cells (monocytes, lymphocytes), for future medical or research use such as precision oncology diagnostic, prognostic and/or drug screening assays for a specific patient after drug resistance and/or disease progression.

Identification of specific cell types in the tumor: A patient-specific tumor avatar was obtained by seeding the different TME cell populations in the 3D mini-tumor, in a ratio compatible to the ratio found in the patient's tumor tissue. In order to find the ratio between the different cell types, particularly the ratio between tumor and stromal cells, a small sample of a tumor tissue or a biopsy was fixed in 4% paraformaldehyde and stained. Epithelial cells and fibroblasts were counted by hematoxylin and eosin (H&E) staining, and/or a molecular biomarker analysis performed by immunohistochemistry. Alternatively, dissociated tumor cells were examined for cell specific biomarkers by flow cytometry analysis.

Tumor tissue or biopsy samples were stained with DAPI, Hoescht, or DRAQ5 to label the nucleus of the cells. Identification of the tumor cells and specific subpopulations among TME cells was performed by staining with at least one antibody, or a plurality of antibodies, directed against a cell surface marker as follows:
a) Pancytokeratin (PanCK), cadherin E and EPCAM for tumor epithelial cells evaluation,
b) CD90, CD73, CD105, CD45, STRO for TA-MSCs evaluation,
c) α-SMA, CAF, FAP, FAP1, PDGF, P4HA1, N-cadherine, Vimentin for TASCs and CAFs evaluation,
d) CD31, MCAM, CD105, CD90, VE-cadherin, vWF, and suprabasin, for TECs evaluation,
e) Perilipin A/B, leptin, HOXC8, HOXC9 for adipocytes evaluation; and,
g) CD4, CD8, CD3+, CD19, CD20, CD56, CD14, CD163, CD68, CD74, CD11b, CD163, CD206 for immune cells evaluation.
Following immunohistochemistry analysis, or flow cytometry analysis, the percentage of each subpopulation in the tumor sample was determined.

Organoid-stroma cells co-culture: Initially, organoids were mechanically dissociated into smaller fragments through pipetting, followed by enzymatic digestion into single cells using dispase II at 37° C. for 20 minutes. Concurrently, stroma cells cultured in FM-2, ECM, and MSCM, as well as Huvec cells cultured in ECM, were harvested at 80% confluency in the flask and dissociated using accutase. In certain experiments, after cells dissociation, each cell population was stained with a vital dye before their co-culture. Cells cultured in ECM and Huvec cell lines were stained blue using the CellTrace Violet Dye Proliferation Kit. Conversely, cells cultured in FM2 medium and organoids were labeled green with the CellTrace CFSE Dye Proliferation Kit or exhibited purple fluorescence upon staining with minclaret Cellvalue® far red fluorescent kit, depending on the experiment's specifications. Imaging was performed at 10× magnification using an Olympus Confocal Microscope. Subsequently, dissociated cells were mixed in 35 ul of co-culture medium, at ratios of 1:1:1, 1:4:1, 4:1:1 and 1:1:4 of Huvec or cells cultured in ECM, cells cultured in FM2 and organoids respectively. Co-cultures composed of ECM:organoids (ORG), FM2:ORG, and Huvec:ORG were also created at 1:1, 1:4, and 4:1 ratios, respectively. Each mix, containing 4000 cells, was then co-cultured in 384 wells low repellent plates, with DMEM/F12 medium supplemented with 10 mM HEPES, abam, 2 mM glutamax, non-essential amino acids, sodium pyruvate, 500 ng ml-1 R-spondin, 100 ng ml-1 noggin, 100 ng ml-1 Wnt3A, 10 nM gastrin, Prostaglandin E2, 100 ng ml-1 IGF1, 10 ng·ml-1 FGF2, 10 ng·ml-1 FGF10, 50 ng·ml-1 EGF, B27, 0.5 μM A 83-01, 4 mM nicotinamide, 10 μM Y-27632, 1 μg ml-1 hydrocortisone, 50 μg ml-1 ascorbic acid, 5 ng ml-1 VEGF and 100 ng ml-1 IL-6, 4 μg ml-1 heparin, and 5% fetal calf serum in 10 ng·ml-1 and fibronectin and 5% Matrigel as scaffold and incubated in 5% CO₂ at 37° C. for different intervals of time.

Tumor Avatar culture: Isolated TEC and TASC cell lines, or isolated CAFs, TA-MSCs, TAAs, and tumor associated immune cells, and cluster of tumor cells from dissociated 3D or 2D tumor epithelial cell cultures were mixed at a ratio comparable to the numeric ratio found in the tumor tissue or biopsy, according to an H&E staining or to the cell surface marker analysis or as a mix at different population ratios from the same patient.

For establishment of the mini-tumor or tumor avatar model, the cell mix was cultured in DMEM/F12 medium supplemented with hepes, abam, glutamax, non-essential amino acids, sodium pyruvate, R-Spondin, Noggin, IGF1, FGF2, FGF10, EGF, VEGF, PDGF, IL-6, IL-8, heparin, intralipids, BMP7, BMP4, Hydrocortisone, ascorbic acid, B27, A 83-01, N-Acetyl-L-cysteine, Nicotinamide, Y-27632 and 5% serum (autologous or AB serum or animal serum).

For gastrointestinal derived tumor avatars, Wnt3A, gastrin and Prostaglandin E2 were added to the medium. For kidney derived tumor avatars, hydrocortisone and epinephrine were added to the medium. For liver derived tumor avatars, HGF was added to the medium. For breast derived tumor avatars, heregulin was added to the medium. For ovary and uterine derived tumor avatars B-Estradiol, hydrocortisone, and heregulin were added to the medium. For bladder cancer derived tumor avatars, FGF7 and heregulin were added to the medium. For lung cancer derived tumor avatars, FGF7 was added to the medium. Fibronectin, Matrigel, or other scaffold combinations were added to the medium. In cases where immune cells were included in the tumor avatars, IL-2, anti-CD3, β mercaptoethanol, and anti-CD28 antibodies were added to the medium to support immune cell proliferation. In cases where adipocytes were included, indomethacin and IBMX, were also added to the medium.

Cells were mixed at selected ratios, suspended in the described medium and seeded in low repellent plates in Matrigel (2-10%) and fibronectin (5-10 ng/ml) scaffold or Matrigel (2-10%) or without scaffold and incubated in 5% CO₂ and 20% or 2% O₂ at 37° C. in a CO₂ incubator. To generate a fluid flow, the culture dishes were placed on a rotatory shaker at 30-70 rpm throughout the culture period. The cells were reorganized as a mini-tumor structure after 1-3 days of culturing and continued growing for at least 1 month after culturing. Tumor avatar cells were dissociated with dispase I or II, sometimes also hyaluronidase and DNase I, or accutase and DNase, or triplex and DNAse at 37° C. during 20 minute-3 hours and cryopreserved. Alternatively, cells were cultured according to the previously described TME and organoid culture protocols.

For drug screening experiments, a single agent or combination of different agents were added to the tumor avatar culture on day 3 of culturing, or when the tumor avatar was first observed to be structurally organized. After 3-7 days of treatment with different drugs or drug combinations, a RealTimeGlo assay was performed. Inhibitory concentration 50 (IC50) and area under the curve (AUC) were calculated to estimate drug responsiveness. The mini-tumor morphology and size were recorded using Operetta Imager.

In order to identify tumor cells in the mini-tumor model, each cell population (e.g., TASCs, TECs, and Org) was labeled with a specific live cell dye (Minclaret, Cell Violet, Cell tracker, DiIC18, SP-DiOC18 or other dyes) with a non-overlapping spectrum and co-cultured to allow cell organization. After treatment cells were subjected to propidium iodide or live or Dye fixable viability staining. Cell death of the specific populations was evaluated after vehicle and drugs treatment by counting CAFs PI+ and PI− cells, TECs PI+ and PI− cells and Org PI+ and PI− cells.

Alternatively, tumor avatars were cultured and treated, and cells mechanically or enzymatically dissociated. Then, cells were labelled using at least one conjugated antibody, or plurality of conjugated antibodies, directed against a specific population cell surface marker such as EPCAM and/or CDHE for epithelial cells, VE-cadherin and/or MCAM and/or CD31 for endothelial cells, N-cadherin and/or PDGFR and/or FAP for TASCs and CAFs. All the antibodies for a specific cell population were conjugated to a fluorochrome with the same absorption/emission spectrum. The antibodies directed against distinct populations were conjugated with non-overlapping spectrum fluorochromes. For identification of positive anti-cancer drugs among the examined drugs, cell death was evaluated using a necrosis marker such as Live or Dye fixable viability staining or PI or DRQ7 together with an apoptotic marker such as annexin with an overlapping spectrum. In some cases, necrosis and apoptosis markers were labelled using non-overlapping spectrums. In some cases, viable cells were also labelled with calcein using a non-overlapping spectrum. In some cases, nuclear staining was performed with DAPI or Hoescht, then, images were acquired with a high throughput imaging system or flow cytometer. The data was analyzed to obtain the amount of live and dead ORG and/or TASCs and/or TECs and/or immune cells after drug treatment as compared to controls. Then inhibitory concentration 50 (IC50) and area under the curve (AUC) were calculated to estimate drug responsiveness.

Cell viability assay: To assess co-culture growth rates and the cytotoxic effects of various drugs, a luminescent cell viability assay was conducted using the CellTiter-Glo kit from Promega following the manufacturer's guidelines. For the drug cytotoxicity assay, co-cultures or organoids were seeded in 384-well plates and allowed to organize for 48 hours. Subsequently, they were treated with chemotherapies, including 5-Fluorouracil (7.5 μM), Leucovorin (0.8 μM), Oxaliplatin (3.5 μM), and Docetaxel (5 μM) (FLOT), or paclitaxel (5 μM), along with targeted therapies such as crizotinib (1 μM), osimertinib (0.8 μM), afatinib (0.150 μM), capmatinib (8 μM), dasatinib (0.180 μM), imatinib (4 μM), palbociclib (0.4 μM), and gefitinib (3 μM) for a duration of 72 hours. The chosen drug concentrations were based on their higher plasma concentration values. Following treatment, each well containing co-cultures or organoids was lysed using CellTiter-Glo reagents, thoroughly mixed, and incubated for 30 minutes, after which luminescence was measured.

Immunofluorescence of stroma cells: The cells were initially fixed in 4% paraformaldehyde for 15 minutes, followed by permeabilization in phosphate-buffered saline (PBS) containing 0.2% Tween-20 at room temperature for 10 minutes. Subsequently, the samples underwent a 30-minute blocking step in a buffer composed of 6% Donkey serum albumin in PBS at room temperature. After blocking, the cells were exposed to primary antibodies, including anti-α-SMA (1:1000), anti-FAP (1:500), anti-EpCAM (1:1000), anti-CD44 (1:75), anti-CD105 (1:600), and anti-CD73 (1:50), diluted in TrueBlack IF blocking buffer containing 1% BSA in PBS, overnight at 4° C. Following the overnight incubation, the samples were washed three times for 15 minutes each with PBS. Next, the samples were incubated with labelled secondary antibodies for 1 hour at room temperature and counterstained using Hoescht (10 ng/ml in PBS). After incubation with the secondary antibodies, the samples underwent another three 15-minute washes in PBS. Finally, the samples were mounted using EverBrite TrueBlack Hardset mounting medium and fluorescence was analyzed using a Zeiss LSM510meta confocal microscope.

Tissue and organoids histology and immunofluorescence: Organoids were initially incubated for 1 hour in cell recovery solution to remove the Matrigel, followed by washing in PBS and fixation in 4% paraformaldehyde for 1 hour. Next, they were embedded in 2% agarose gel. Tumor tissue underwent dissection into 4% neutral buffered formalin for 12 hours. Both organoids and tissue were incubated for 24 hours in ethanol and embedded in paraffin blocks using a standard histological protocol. The sections were then permeabilized and blocked using TrueBlack IF buffers for 15 minutes before incubation with primary antibodies overnight. These primary antibodies included anti-MCAM (1:100), anti-vimentin (1:200), anti-EpCAM (1:100), anti-ALDH1 (1:100), anti-CD133 (1:600), anti-E-cadherin (1:1000), anti-CD44 (1:75), anti-CD105 (1:600), and anti-CD73 (1:50). Subsequently, the slides were incubated with labelled secondary antibodies for 1 hour at room temperature and counterstained using Hoescht (10 ng/ml) in PBS). After washing the samples 3 times of 15 minutes each in PBS, they were mounted using EverBrite TrueBlack Hardset mounting medium. Subsequently, fluorescence analysis was conducted using a Zeiss LSM510 confocal microscope. Organoid and tissue H&E staining was conducted manually following a standard staining protocol. H&E images were obtained using a Leica microscope.

Flow cytometry analysis: For flow cytometric analysis, stromal cells cultured in ECM, FM2, or MSCM media were detached using trypsin, washed, and resuspended in Cell Staining buffer. They were then blocked for 10 minutes with TruStain FC blocker. Following this, the cells underwent surface staining by incubating the samples for 1 hour using a panel consisting of APC/Fire 750 anti-human E-Cadherin (1:20), Alexa Fluor 700 anti-human FAP (1:15), Brilliant Violet 421 anti-human CD146 (1:20), VioBlue anti-human CD144 (1:20), PE anti-N-Cadherin (1:50), Alexa Fluor 647 anti-human EPCAM (1:15), Brilliant Violet 785 anti-human CD90 (1:20), APC anti-human CD105 (1:20), PE/Cyanine7 anti-human CD140a (1:20), and PerCP/Cyanine 5.5 anti-human CD31 (1:15) antibodies. Additionally, Brilliant Violet 421 anti-human CD73 (1:20) antibodies were also included in the analysis. Cells were washed in PBS and fluorescent data were collected using a Cytoflex Flow cytometer. Subsequently analyzed using FlowJo software version 10.8.1.

Whole-exome and transcriptome sequencing: Whole-genome DNA and RNAseq samples were prepared using the Allprep DNA RNA protein mini kit in accordance with the manufacturer's instructions. DNA exome sequencing and RNA sequencing were conducted on gastric cancer tumor and normal tissue, as well as on organoids (passage (P) 7) and stroma cells cultured in FM2 (P8), ECM (P5, P6), and MSCM (P6, P7) mediums. Additionally, transcriptome sequencing was performed on co-cultures comprising organoids and cells cultured in FM2 and ECM mediums at various ratios, and on co-cultures composed of organoids, cells cultured in FM2, and Huvec cells at different ratios.

Whole exome DNA-sequencing: Targeted library preparation, DNA-sequencing and bioinformatic analysis were outsourced to BGI Genomics Co Ltd (Hong Kong, China). In brief, lug of genomic DNA underwent fragmentation using the Covaris LE220-plus focused ultrasonicator (Covaris LLC, Woburn, MA, USA). Subsequently, the fragmented DNA was employed for library preparation using the Agilent V5 exome kit. This involved various steps such as end-repair, A-tailing, adaptor ligation, and amplification. The sequencing libraries were then subjected to end sequencing on a DNBSEQ platform (BGI Genomics Co. Ltd, Hong Kong, China) with an average coverage depth >30×. Finally, the data was processed into raw data in FASTQ format. Raw sequencing reads underwent filtration to eliminate low-quality data, leaving behind clean reads. These clean reads were then aligned to the hg19 human reference genome using the Burrows-Wheeler aligner (BWA, v0.7.17). To ensure precise variant calling, the recommended Best Practices for variant analysis using the Genome Analysis Toolkit (GATK) were used. The resulting BAM file served as input for calling SNVs/indels using the HaplotypeCaller of GATK (v4.0.11), identifying CNVs and structural variants via BreakDancer, and detecting copy number variation using CNVnator. COSMIC database was used to identified somatic mutations in GC (cancer.sanger.ac.uk/cosmic). Driver mutations were determined using the Cancer Genome Interpreter (CGI). Oncogenes were identified using OncoKb platform (oncokb.org/). Data was analyzed and visualized using Phython.

RNA sequencing: RNA sequencing and data analysis were outsourced to The Nancy and Stephen Grand Israel National Center for Personalized Medicine (Israel). RNA sequencing Libraries were prepared using LIB_PREP_PROTOCOL. READ TYPE reads were sequenced on NUM_LANES lane(s) of an Illumina ILLUMINA_INSTRUMENT. The output was ˜26 million reads per sample. Poly-A/T stretches and Illumina adapters were trimmed from the reads using cutadapt; resulting reads shorter than 30 bp were discarded. Reads were mapped to the H. sapiens reference genome GRCh38 using STAR, supplied with gene annotations downloaded from Ensembl and outFilterMismatch NoverLmax was set to 0.04. Reads with the same UMI were removed using the PICARD MarkDuplicate tool using the BARCODE_TAG parameter. Expression levels for each gene were quantified using htseq-count, using the gtf above. Differentially expressed genes were identified using DESeq2 with the betaPrior, cooksCutoff and independentFiltering parameters set to False. Raw P values were adjusted for multiple testing using the procedure of Benjamini and Hochberg. Pipeline was run using snakemake. The log 2FC values of all genes were used for hierarchical clustering using Euclidean method. The clustering results were visualized as a heatmap using R.

Pathway and gene set enrichment analysis: Genomic gene set enrichment analysis was performed for germline and somatic mutations of analyzed samples. Transcriptomic gene set enrichment analysis was performed using the significantly up or downregulated genes in each comparison group (log 2FoldChange (FC)=1, p=0.05, n=30) to identify the molecular signatures. To identify significantly enriched pathways between comparison groups, differentially expressed genes were analyzed using WEB-based Gene SeT Analysis Toolkit (webgestalt.org) analyzing Gene Set Enrichment Analysis in Reactome, KEEG, Panther and Wikipathways cancer databases. Significant pathways are selected from the top 20 gene sets with a false discovery rate q-value of less than 0.05. Analyzed data was visualized using Python.

Drug selection based on gene expression: Drug and drug target relationship of genes meeting criteria (log 2FC=1, p=0.05, n=30) and found to be upregulated in organoids compared to cells cultured in FM2, ECM and MSCM mediums, were obtained from DrugBank portal (go.drugbank.com/). Subsequently, constant inhibition or IC50 values of genes associated with each identified drug were sourced from DrugTargetCommons (drugtargetcommons.fimm.fi/) or PubChem (pubchem.ncbi.nlm.nih.gov) platform databases. Moreover, evidence regarding the involvement of these genes in gastric cancer oncogenesis and survival was gathered from PubMed (pubmed.ncbi.nlm.nih.gov/).

Statistical analysis: Statistical analyses were conducted using Excel, with data presented as means±SE. Gene expression comparisons were performed using one-way analysis of variance (ANOVA) to assess differences between groups.

### Example 1: Isolation and Expansion of Paired Human Tumor Organoids and Associated Stroma Cells

To create a tumoroid model with stromal components, tumor epithelial and stroma cells were extracted from a 59-year-old female diagnosed with poorly differentiated, intestinal type, advanced metastatic stomach adenocarcinoma. Organoids were cultured in four different mediums using standard protocols (Table 1). After four days of cultivation, a higher number of organoids with larger diameters and a discernible cystic morphology were observed using medium 3 as opposed to the other mediums (FIG. 1B-1E). Immunofluorescence stained showed elevated organoid expression of the epithelial cell marker cadherin E (E-CDH), and the stemness markers CD133 and ALDH1, but low expression levels of the mesenchymal stem cells markers CD73 and CD105 (FIG. 1F).

To consider the heterogeneity of stroma cells, tumor derived stroma cells were cultured in different mediums. Initially, the cells were cultured in MSCM. Following a three-week cultivation period, when cells reached confluency, they were sub-cultured in either ECM or FM2 and sustained in these media throughout subsequent subcultures. These cells adhered as a monolayer and exhibited elongated morphology (FIG. 1G-1J). Their characterization via immunofluorescence staining and flow cytometry analysis revealed that over 95% of the cells expressed the stroma cell marker vimentin as well as the mesenchymal stem cells markers CD105, CD90 and CD73 across all mediums. Notably, cells cultured in ECM displayed significantly elevated levels of the endothelial cells related markers CD146 (MCAM) and VE-cadherin compared to those grown in MSCM and FM2. However, no CD31 expression was observed in any of the cells. Importantly, there was no detectable expression of the epithelial markers, EPCAM and E-cadherin, in cells cultured in MSCM, ECM or FM2 media.

TABLE 1
Media tested for culturing tumor avatar/organoid (Bartfeld refers to “In vitro
expansion of human gastric epithelial stem cells and their responses to bacterial infection.
Gastroenterology”; Fujii refers to “Human Intestinal Organoids Maintain Self-Renewal
Capacity and Cellular Diversity in Niche-Inspired Culture Condition”; Vlachogiannis refers
to “Patient-derived organoids model treatment response of metastatic gastrointestinal
cancers” all of which are hereby incorporated by reference in their entirety.)

| | Reference | | | | | | | |
|---|---|---|---|---|---|---|---|---|
| | Bartfeld et al | | Fujii et al, | | Vlachogiannis | | M1 + | |
| | 2015 | | 2018 | | et al; 2018 | | M2 + M3 | |
| Media Composition | Medium 1 | | Medium2 | | Medium 3 | | Medium 4 | |
| Advanced DMEM/F12 | Yes | | Yes | | Yes | | Yes | |
| Hepes | 10 | mM | 10 | mM | 10 | mM | 10 | mM |
| ABAM | 1× | | 1× | | 1× | | 1× | |
| GlutaMax Supplement | 2 | mM | 2 | mM | 2 | mM | 2 | mM |
| B-27 Supplement | 1× | | 1× | | 1× | | 1× | |
| R-spondin 1 | 250 | ng · ml⁻¹ | 500 | ng · ml⁻¹ | 500 | ng · ml⁻¹ | 500 | ng · ml⁻¹ |
| Noggin | 100 | ng · ml⁻¹ | 100 | ng · ml⁻¹ | 100 | ng · ml⁻¹ | 100 | ng · ml⁻¹ |
| Wnt3a | 50 | ng · ml⁻¹ | 50 | ng · ml⁻¹ | 100 | ng · ml⁻¹ | 50 | ng · ml⁻¹ |
| EGF | 50 | ng · ml⁻¹ | 50 | ng · ml⁻¹ | 50 | ng · ml⁻¹ | 50 | ng · ml⁻¹ |
| FGF-2 | No | | 50 | ng · ml⁻¹ | 10 | ng · ml⁻¹ | 50 | ng · ml⁻¹ |
| FGF10 | 200 | ng ml⁻¹ | No | | 10 | ng · ml⁻¹ | 10 | ng · ml⁻¹ |
| IGF1 | No | | 100 | ng ml⁻¹ | No | | 100 | ng · ml⁻¹ |
| Gastrin | 1 | nM | 10 | nM | 10 | nM | 10 | nM |
| Prostaglandin E2 | No | | No | | 1 | μM | 1 | μM |
| A 83-01 | 2 | μM | 0.5 | μM | 0.5 | μM | 0.5 | μM |
| Nicotinamide | 10 | mM | No | | 4 | mM | 10 | mM |
| N-Acetyl-L-cysteine | 1 | mM | 1 | mM | No | | 1 | mM |
| SB 202190 | No | | No | | 5 | μM | 5 | μM |
| Y-27632 | 10 | μM | 10 | μM | 10 | μM | 10 | μM |
| Caspofungin | 0.5 | μg/ml | 0.5 | μg/ml | 0.5 | μg/ml | 0.5 | μg/ml |

### Example 2:Organoid and Stroma Cells Recapitulate the Mutational Spectrum of Primary Tumor Tissue

To identify germline and somatic mutations, whole exome sequencing was conducted on the parental tumor, adjacent normal stomach tissue, as well as on organoids and tumor associated stroma cells cultured in MSCM, ECM or FM2 media.

The patient carried germline pathogenic mutations in IL4R gene, known to be linked with increased susceptibility to gastric adenocarcinoma. Additionally, the patient exhibited likely pathogenic germline mutations in NBN and MUTYH genes, known for their involvement in cancer susceptibility. Further likely pathogenic germline mutations were identified in METTL8, PDE4DIP, TMEM260, CX3CR1, BTD, and EML3 genes. Furthermore, benign germline mutations with moderate and high impact were observed in genes associated with increased susceptibility to GC, such as CDH1, P53, BRCA1, BRCA2, MLH1, MSH2, MSH6, PMS2, ATM, APC, PALB2, BRIP1, and EPCAM.

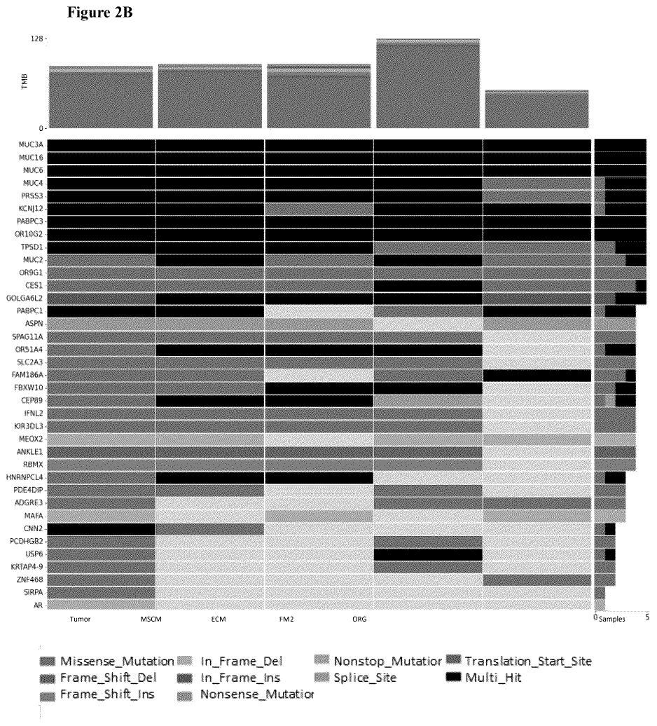

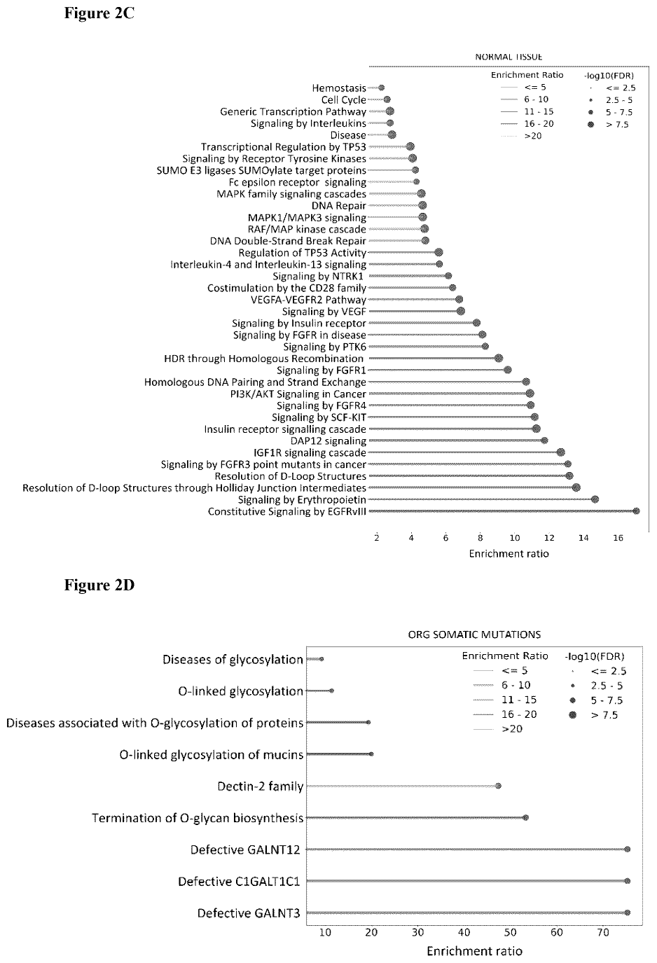

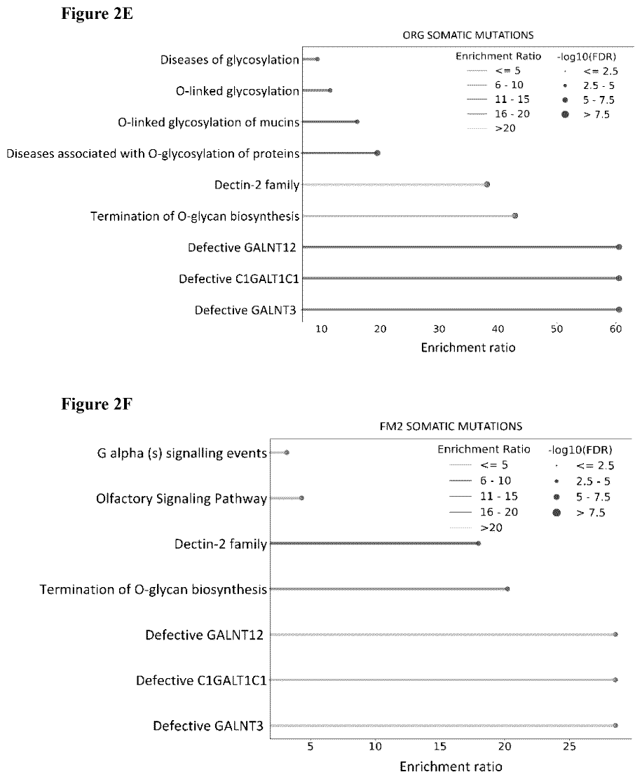

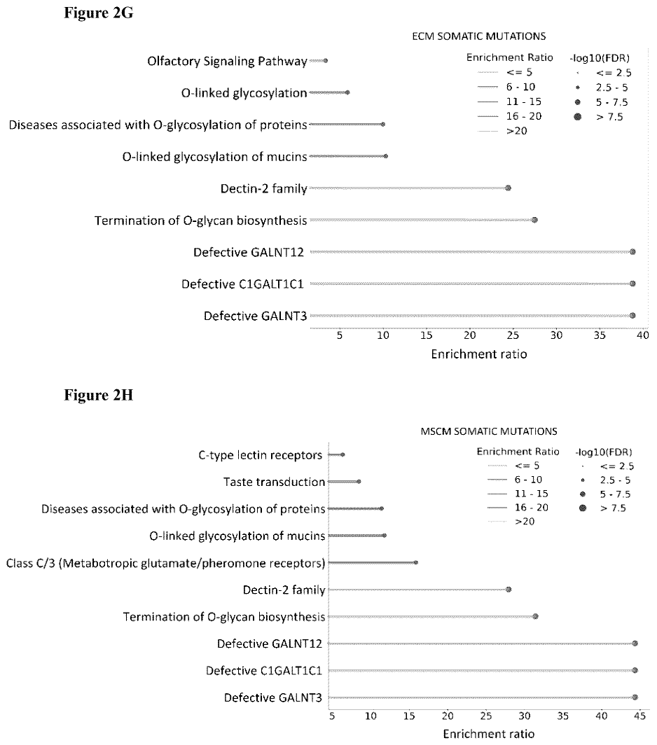

Mutational profile comparison between organoids, MSCM, ECM and FM2 cultured cells with their respective parental tumors, revealed a 98.5-99% level of concordance for each cell type, underscoring the close similarity in their genetic data. These results supported the overall genetic representativity of the organoids and stroma cells to their parental tumors (FIG. 2A). By filtering out mutations present in normal tissue and those with low impact, 19 commonly mutated genes were identified at the intersection of somatic mutations between the tumor and derived cultured cells (FIG. 2B). Moreover, each sample exhibited unique mutated genes, with specific mutations shared among certain samples. Interestingly, the number of novel mutations was notably higher in cells cultured in MSCM, FM2 and ECM mediums as compared to organoids. However, none of these mutations demonstrated pathogenic clinical significance.

Remarkably, the mutational profiles of the patient-derived cultured cells mirrored prevalent genomic alterations seen in gastric adenocarcinoma. Analysis of the somatic mutations using the COSMIC database revealed 37 non-pathogenic somatically mutated genes in the parental tumor, with an uneven distribution between organoids and tumor associated stroma cells cultures (FIG. 2C). Notably, neither germline nor somatic variants in the patient's mutated genes were identified as potential therapeutic targets, underscoring the absence of actionable treatment options.

Functional analysis of the organoids and stroma cells mutational landscape using gene ontology terms and the Reactome database revealed implications of germline mutated genes in metabolomics, cell cycle regulation, DNA repair mechanisms, and signaling pathways involving tyrosine kinase receptors (IGF1R, FGFRs, VEGFR, NTRK), as well as PI3K, AKT, and MAPK pathways (false discovery rate (FDR)<0.05). Additionally, somatic mutations affected, glycosylation processes and pathways enriched in mucins proteins (FDR <0.05) (FIG. 2D-2H). Notably, both tumor and cultured cells exhibited mutations in genes associated with olfactory pathways.

### Example 3:Organoids and the Different Stroma Cells Subtypes Display Distinct Gene Signatures

To gain insights into the gene expression signatures of the different types of tumor-derived cultured cells, RNA sequencing was performed. Differentially expressed genes (DEGs) between organoids and cancer associated stroma cells cultured in MSCM, ECM, and FM2 mediums were identified, requiring an expression log 2 fold change of more than 1, an adjusted p-value <0.05, and n counts >=30. This analysis revealed 2325 DEGs out of a total of 20384 genes, with 1192 up-regulated and 1133 down-regulated in the organoids compared to stroma cells (FIG. 3A). Pearson correlation analysis of the transcriptome data indicated significant positive correlations among different stroma cells (p=0.94), but a lower correlation was observed between organoids and stroma cells (p=0.7).

Gene set enrichment analysis (FDR<0.05) using the Webgestalt Toolkit revealed distinct biological pathways associated with upregulated genes in organoids and stroma cells. Top pathways in organoids included glycosylation of mucins, formation of the cornified envelope, Rap1 signaling pathway, glycosphingolipid biosynthesis, and cell junction molecules (FIG. 3B). In contrast, stroma cells exhibited higher expression of genes involved in extracellular matrix organization, collagen formation, integrin cell surface interactions, NCAM1 interactions, complement and coagulation cascades, interleukin signaling, and PDGF, MET, Receptor Tyrosine Kinases, and PI3K-Akt signaling pathways (FIG. 3C).

Gene expression comparison between organoids and stroma cells using the OncoKb database identified 29 oncogenes with higher expression in organoids (log 2FC>1, p=0.05), and 34 oncogenes upregulated in stromal cells (log 2FC>1, p=0.05) (FIG. 3D). Notably, both organoids and stroma cells oncogenes were enriched in receptor tyrosine kinase signaling related to PI3K/AKT and RAF/MAPK signaling. Organoids oncogenes related to GC included FGFR2, FGFR3, FGF19, ERBB2, ERBB3, MET, and PDGFB. Other organoids oncogenes encompassed B and T cell receptor signaling pathways (CARD11, KRAS, SYK, VAV1, VAV2), NF-kappa B signaling (CARD11, LTB, SYK), and cAMP signaling (CACNA1D, SOX9, VAV1, and VAV20). Stroma cells oncogenes involved angiogenesis-related genes (AKT3, ETS1, FGFR1, HIF1A, JAK1, PDGFRA, and PDGFRB), interleukin signaling (IL6, IL7, IL27, and IL35), and RUNX2 signaling pathways (ABL1, AR, and COL1A1).

The examination of genes associated with specific cell types revealed elevated expression levels of epithelial markers such as EPCAM, CDH1, KRT18, and AQP5 in organoids, and increased expression of stroma cell markers including COL1A1, COL1A2, COL3A1, COL5A1, EML1, FN1, ITGA4, PDPN, SERPINE 1, SPARC, TSPAN9, PDGFRA, and PDGFRB across all the stroma cell cultures (FIG. 3E).

Comparison of cells cultured in ECM, FM-2, and MSCM mediums revealed specific transcriptomic profiles for each group characterized by differentially upregulated and downregulated genes within each population. (FIG. 3F-3G). FM2 cultured cells exhibited enrichment in cholesterol, lipids, steroids, and sphingolipids metabolism compared to ECM, and in triglyceride metabolism and transcription signaling compared to MSCM cultures (FIG. 3H-31). Conversely, ECM and MSCM cultured cells were enriched in genes related to cell cycle signaling, interleukins (IL4R, IL7R, IL6, IL17, IL18 and IL27RA), MAPK, PI3K-Akt, p53, TNF, Hippo, TGF-beta, and NOTCH1 signaling pathways. Moreover, there was an upregulation of genes regulating pluripotency of stem cells, and genes involved in integrin and non-integrin cell surface interactions, elastic fiber formation, dissolution of fibrin clot, plasminogen activating cascade, and cell surface interactions at the vascular wall compared to FM2 cultured cells (FIG. 3J-3K). MSCM cultured cells showed enrichment in genes related to Th1, Th2, and Th17 cell differentiation and hematopoietic cell lineage compared to cells cultured in FM2 and ECM mediums.

Distinct subtypes of CAFs have been identified in gastric cancer (GC), including inflammatory CAFs (iCAFs) and extracellular matrix-remodeling CAFs (eCAFs). iCAFs interact with T cells and express inflammatory factors and chemokines, contributing to chemotherapy-resistant phenotypes in GC, while eCAFs hinder chemotherapy access to the tumor, correlating with poor prognosis. CAFs cultured in MSCM and ECM media displayed upregulation of pro-inflammatory pathways and hypoxia-related genes, indicating an iCAF phenotype. In contrast, cells cultured in FM2 medium exhibited activation of lipid and steroid pathways. As cancer progresses, CAFs undergo lipidomics reprogramming, affecting tumor metabolism, proliferation, and invasion, and shift cytokine secretion toward an immunosuppressive microenvironment. Addition of both types of CAFs to the tumor avatar produce a more accurate picture of the cancer itself and improve the ability to predict drug response/resistance.

### Example 4: Construction of 3D Co-culture Systems of Gastric Tumor Organoids and Associated Stroma Cells

An aim of the current endeavor was to create a 3D organotypic model where gastric tumor organoids and stroma cells coexist, allowing to simulate the complex interplay found within tumor environments. To replicate the tumor environment in an ex vivo culture model, organoids and cultured stroma cells derived from the same tumor tissue were co-cultured on low-repellent plates on a scaffold comprising matrigel and fibronectin. Furthermore, diverse cell seeding ratios were utilized, including 1:1:1, 1:4:1, 4:1:1, and 1:1:4 for ECM-cultured cells, FM2-cultured cells, and organoids, respectively. To promote endothelial cell survival, the medium was supplemented with VEGF and IL-6, while hydrocortisone, ascorbic acid, sodium pyruvate, and heparin were included to support fibroblast culture.

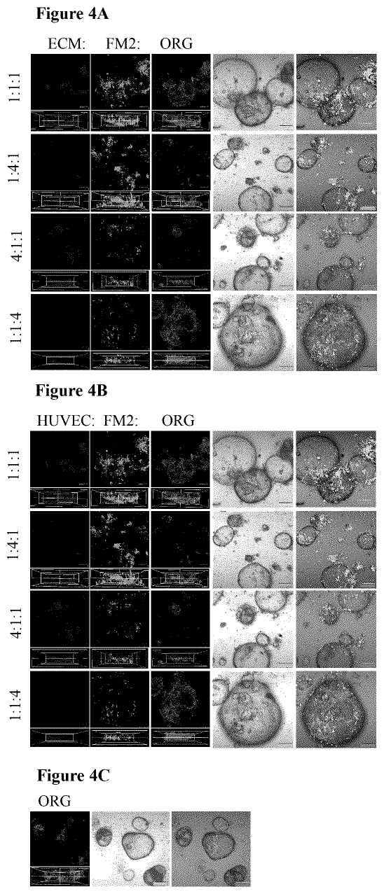

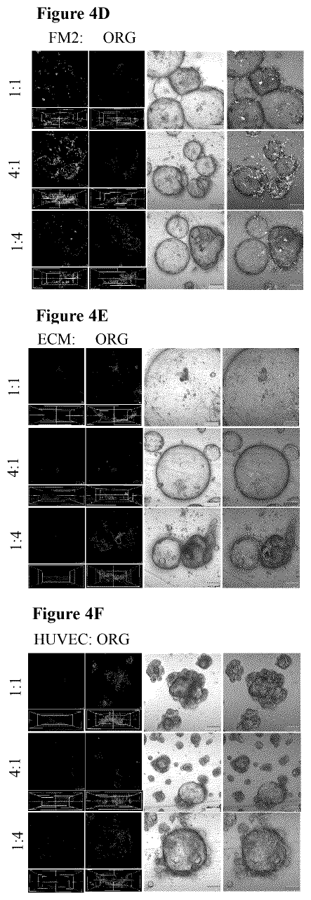

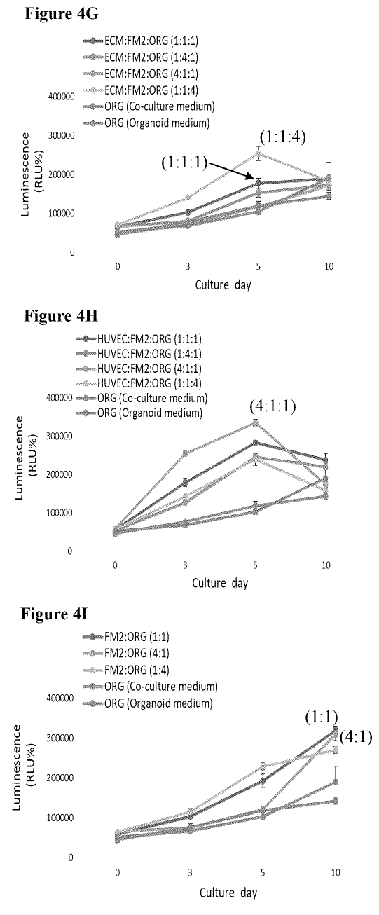

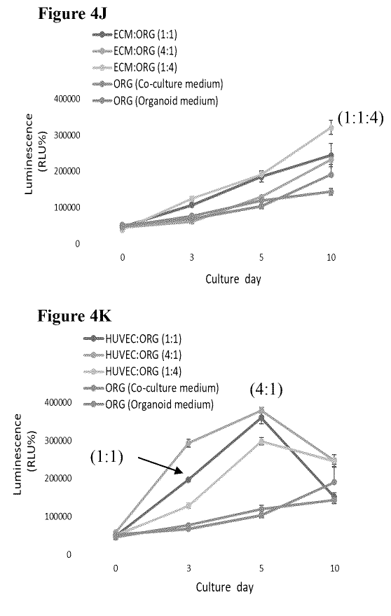

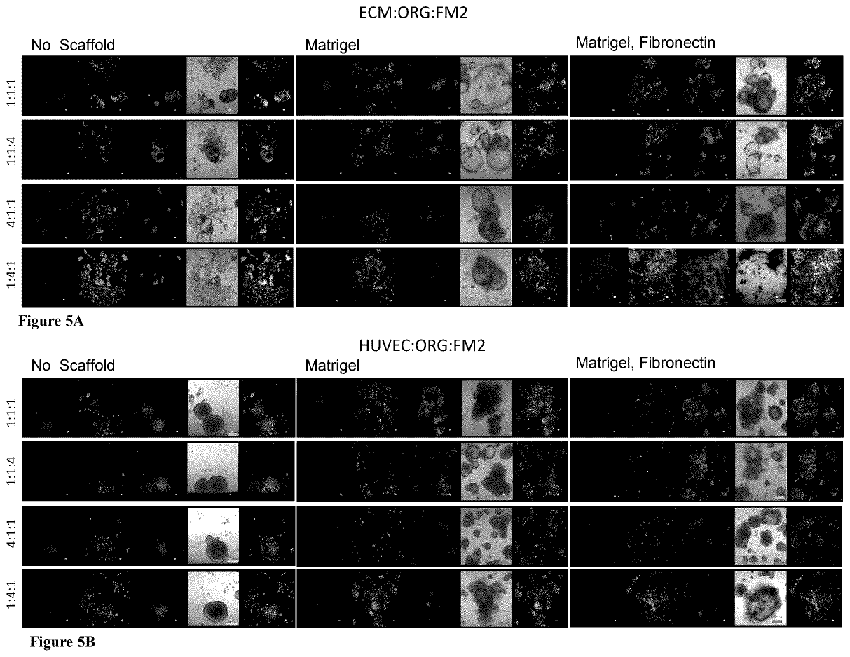

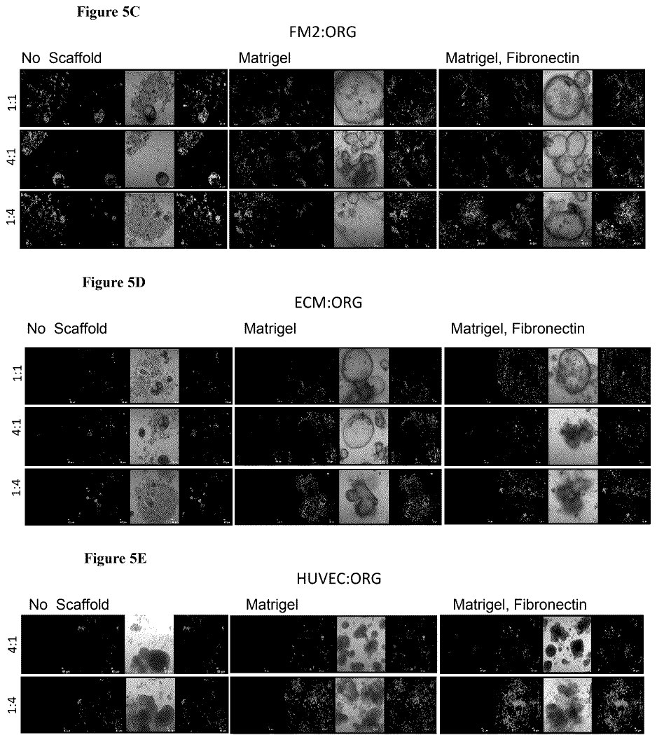

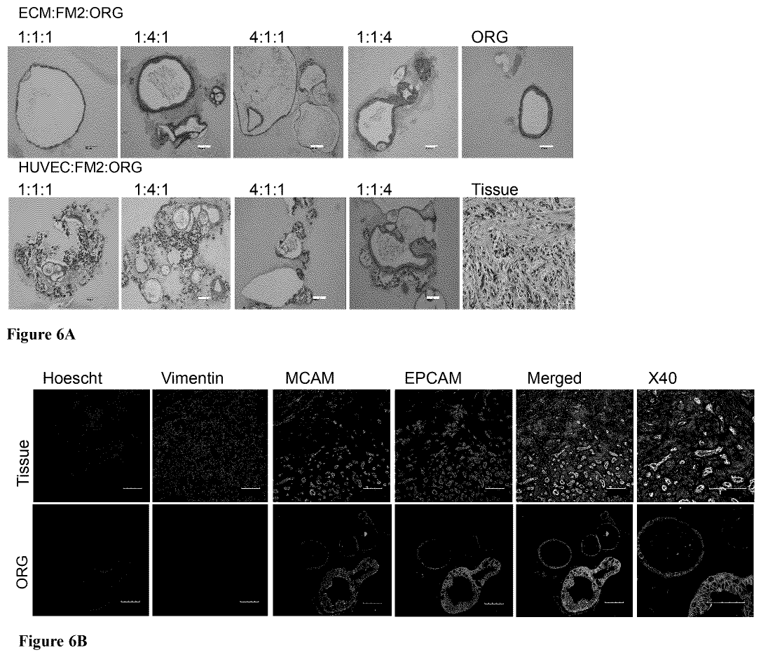

In this study, a multi-color staining method was employed to distinguish between various cell populations within the co-culture system. The use of distinct vital colors for each culture type facilitated the clear identification of individual cell types amidst the complex cellular milieu (FIG. 4A-4F). Even after 5 days of culture, it was observed that the relative proportions of the various cell populations remained consistent with the initial seeding ratios. However, alongside the successful discrimination of cell subpopulations, there was also noted the presence of unstained cells, possibly due to dye loss over successive replications.

Cells co-cultured in this manner arranged themselves into bigger organoid like bubble 3D structures. Particularly, configurations with ratios of 1:1:1 and 1:1:4 exhibited a more rapid organization into larger structures. Co-cultures consisting of Huvec, cells cultured in FM2, and organoids at comparable ratios organized into aggregates in a more condensed spatial structure. However, co-cultures with ratios of 1:1:1 and 1:1:4 still displayed quicker organization and growth. Both types of co-cultures exhibited larger sizes compared to organoids cultured with the same initial number of cells (FIG. 4A-4C).

Co-cultures comprising only one type of stromal cells formed structures akin to those with three types, with co-cultures at 1:1 or 1:4 ratios exhibiting faster growth and organization compared to those with higher proportions of stromal cells (FIG. 4D-4E). On the other hand, the presence of Huvec cells still resulted in a more condensed 3D structure (FIG. 4D-4F).

Cell growth was assessed at various time points throughout the culture period using the CellTiter-Glo assay. ECM:FM2:ORG mixes with a higher proportion of organoids, specifically at ratios of 1:1:1 and 1:1:4, exhibited faster growth compared to those with ratios of 1:4:1 and 4:1:1 and to organoids (FIG. 4G). Similarly, co-cultures of FM2:ORG and ECM:ORG at 1:1 or 1:4 ratios respectively grew faster than those with a higher proportion of stromal cells at a ratio of 4:1 (FIG. 4I-4J). Interestingly, after 10 days of culture, compositions with two types of cells showed higher viability values than their corresponding tri-cultures. Conversely, the growth rate of Huvec:FM2:ORG and Huvec:ORG co-cultures was initially faster but decreased after 10 days of culture. Moreover, cultures with a higher proportion of Huvec cells exhibited greater growth, as evidenced at ratios of 4:1:1 and 4:1 (FIG. 4H, 4K).

The impact of the scaffold on co-culture organization was then investigated by seeding the co-culture under different conditions: without a scaffold, with a matrigel scaffold, or with a combination of matrigel and fibronectin (FIG. 5A-5E). Notably, in the absence of a scaffold, there was no aggregation of ECM:FM2:ORG, FM2:ORG and ECM:Org co-cultured cells, whereas Huvec:FM2:ORG and Huvec:ORG co-cultured cells organized into a spheroid-like structure. After 7 days of seeding, cells co-cultured in the presence of a matrigel scaffold exhibited enlarged sizes compared to organoids and showed an organoid-like structure, while those also containing Huvec:FM2:ORG cells organized into aggregates, with no observable differences in the spatial organization of the co-cultures grown in matrigel alone or in combination with fibronectin. However, the inclusion of fibronectin led to a notable increase in the abundance of FM2-cultured cells within the co-culture.

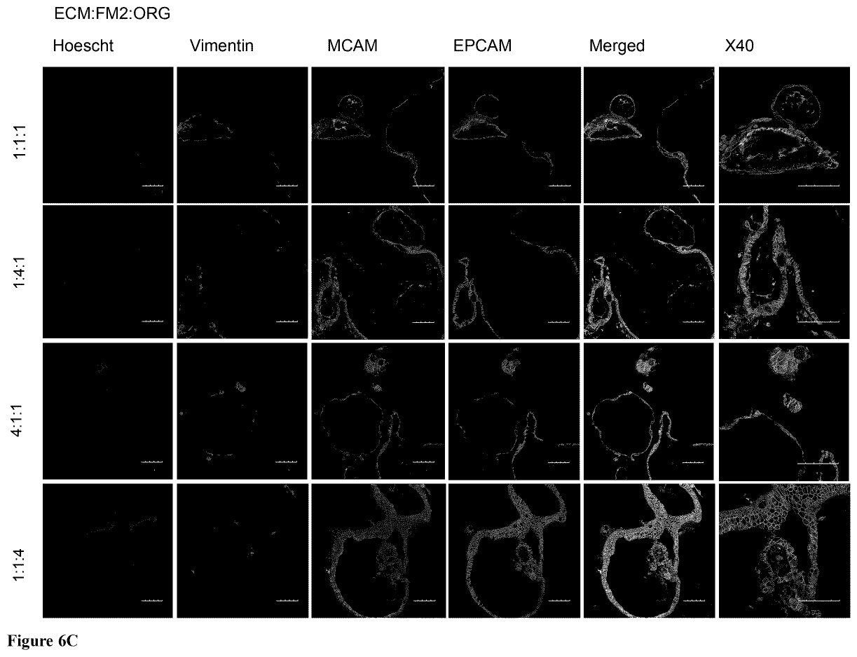

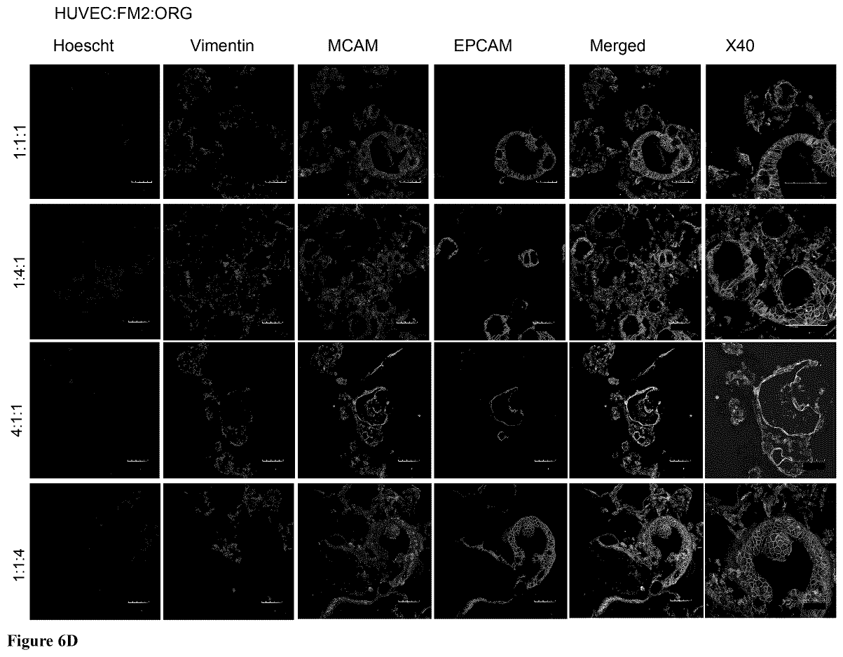

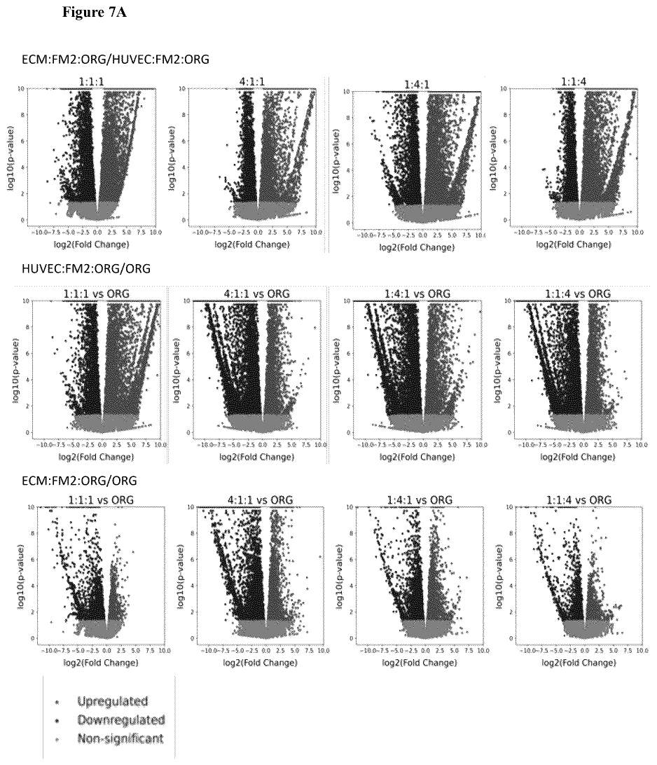

Immunofluorescence analysis of the primary tissue, and the co-cultures cells displayed the presence of tumor epithelial cells, characterized by positive staining for the epithelial EPCAM protein, and of stroma cells characterized by positive staining for vimentin and MCAM directed antibodies. In contrast, the organoids were negative for vimentin staining. Huvec:FM2:ORG co-cultures, showed higher protein expression of vimentin as compared with ECM:FM2:ORG co-cultures (FIG. 6A-6E).

### Example 5: Induction of Specific Gene Expression by Co-Culture of Tumor Organoids with Associated Stroma Cells

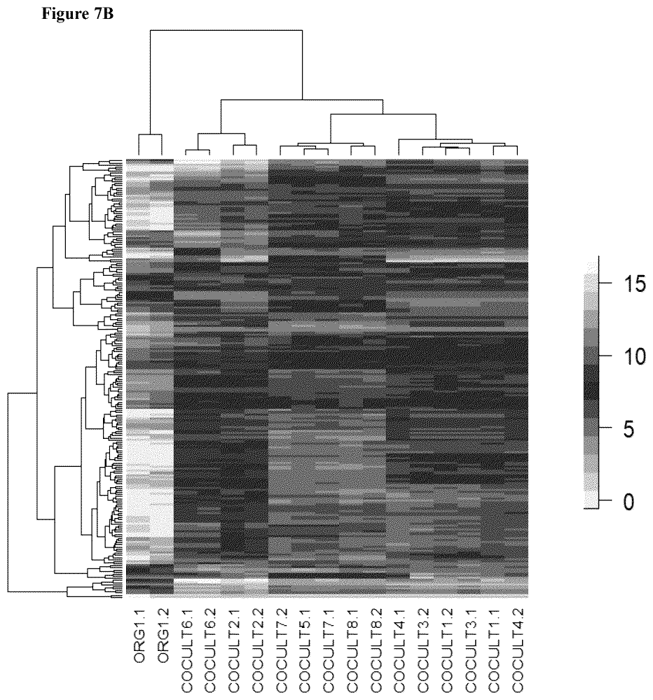

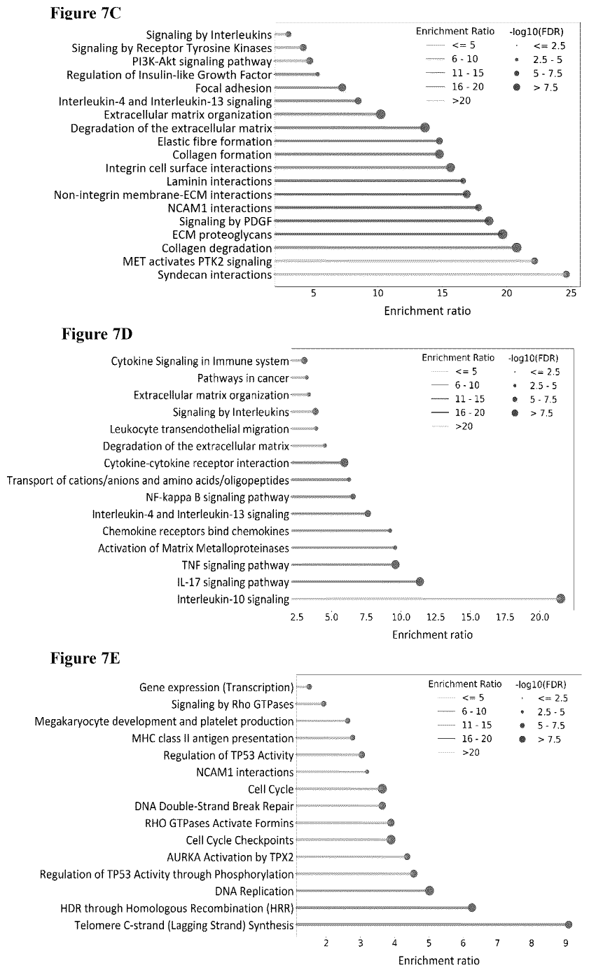

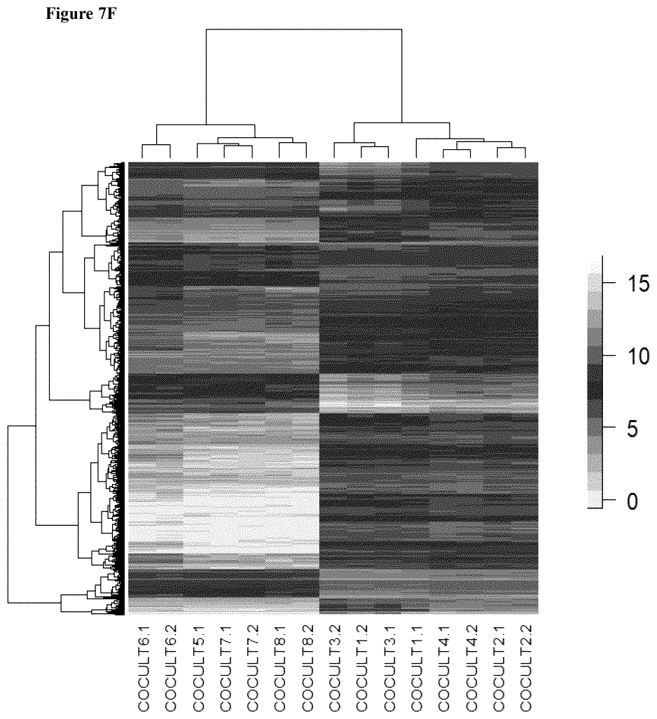

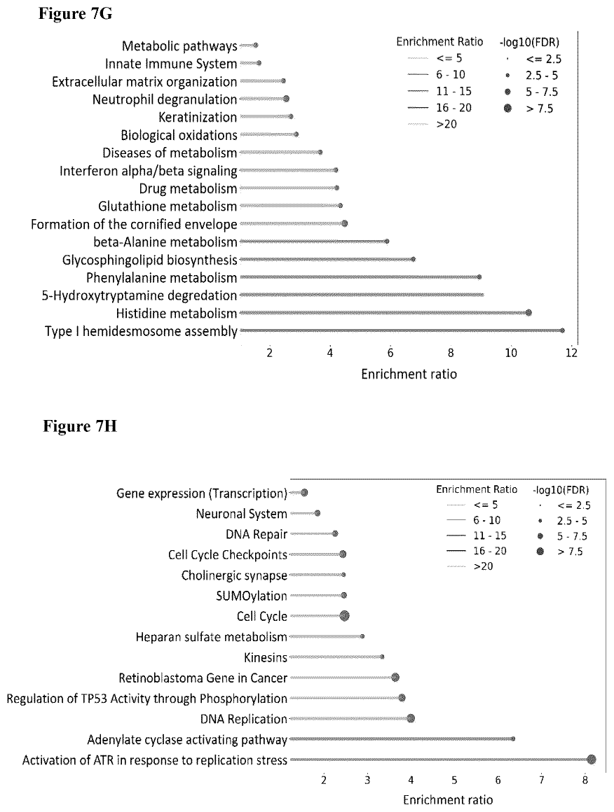

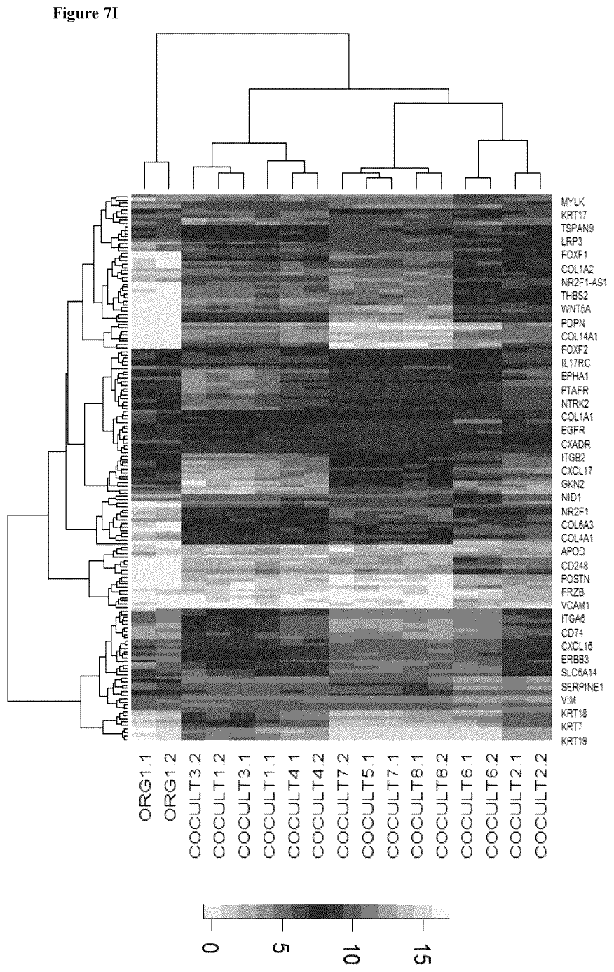

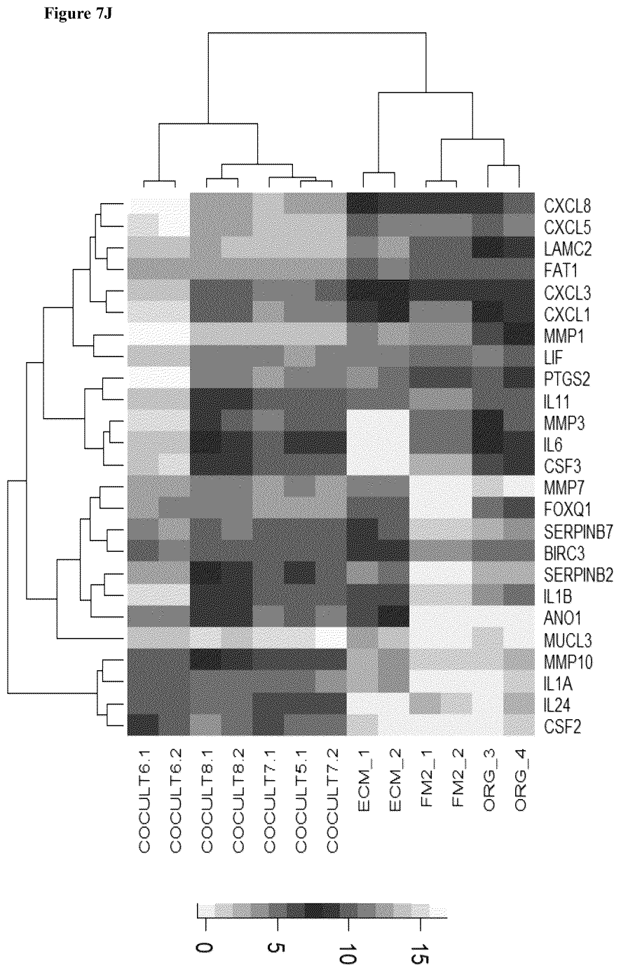

Analysis of gene expression patterns comparing organoids, stroma cells, and their co-cultures by volcano plots revealed significant differences between ECM:FM-2:Org and Huvec:FM-2:Org co-cultures, despite maintaining equivalent cell ratios. The number of genes exhibiting differential expression was notably lower in ECM:FM-2:Org than in Huvec:FM-2:Org co-culture, when compared to organoids cultured. Furthermore, within each type of co-culture (ECM:FM-2:Org or Huvec:FM-2:Org), the variations in cell ratios studied exhibited the fewest differentially expressed genes, underscoring the intricate interplay between cellular compositions and their transcriptional profiles within co-culture environments (FIG. 7A).

Hierarchical clustering heat maps and gene enrichment analysis highlighted DEGs between the organoids and co-cultures (FIG. 7B-7C). Specifically, the co-cultures models expressed higher expression levels of genes associated with extracellular matrix organization and degradation (e.g., collagen, laminin, elastic fiber, syndecam, proteoglycans), integrin and non-integrin cell surface interactions, as well as signaling pathways involving PDGF (PDGFRA, PDGFRB), MET (HGF), PI3K-Akt (FGF2, FGFR1, AKT3), and pro-inflammatory related genes including IL1α, IL4, IL6, IL10, IL11, IL18, IL24, IL33, and CXCL8. Notably, there was a significant gene expression similarity observed between ECM:FM-2:Org and Huvec:FM-2:Org co-cultures containing a higher ratio of FM2 co-cultured cells, when compared to organoids.

Gene expression profiles comparisons of DEGs between ECM:FM-2:ORG or Huvec:FM-2:ORG and organoids together with cells monocultured in MSCM, ECM, and FM2 mediums was conducted. Co-cultures with ECM culture cells displayed higher expression of genes primarily associated with a pro-inflammatory immune response and extracellular matrix remodeling signaling (FIG. 7D). In contrast co-cultures containing Huvec cells exhibited higher expression of genes mainly related to DNA replication, DNA repair and cell cycle (FIG. 7E).

Comparisons of DEGs between ECM:FM-2:ORG and Huvec:FM-2:ORG co-cultures revealed that ECM:FM-2:ORG had higher expression of genes associated with epithelial cells including those involved in keratinization and formation of the cornified envelope together. Additionally, genes related to immune response, metabolomic pathways, and extracellular matrix organization were also more prominently expressed in ECM:FM-2:ORG. Conversely, Huvec containing co-cultures exhibited activation of DNA and cell cycle related genes (FIG. 7F-7H).

Study of genes related to specific cells displayed expression of epithelial markers such as EPCAM, CDH1, KRT18, AQP5 as well as stroma cells markers including COL3A1, COL5A1, PDPN, SERPINE 1, PDGFRA and PDGFRB in each of the analyzed co-cultures, indicating the preservation of different cell types during co-culture. However, the expression of epithelial markers was significantly lower in Huvec:FM-2:ORG co-cultures (FIG. 7I).

The direct interaction between tumor and stroma cells led to a differential transcriptomic profile of the co-culture that encompassed both organoids and stroma cells gene signatures, along with the expression of genes that were low or not present in either of the monocultures. In particular, the pro-inflammatory cytokine IL1β, and the plasminogen activator inhibitor type 2 SERPINB2 genes were highly expressed across all the co-cultured models and had elevated fold change expression respect to the monocultures. Additionally, up-regulation of chemokines (CXCL3, CXCL5, CXCL8), pro-inflammatory related genes (PTGS2 (COX-2), CSF3), extracellular matrix related genes (LAMC2, MMP1, MMP3, MMP7), the inhibitory of apoptosis BIRC3 gene, the voltage-gated calcium-activated anion channel ANO1 and the oncogenic transcription factor FOXQ1 was observed upon co-culture.

Moreover, co-cultures with an elevated ratio of FM2 culture cells, induced higher expression of additional genes including IL1α, IL6, LIF, IL11, IL24, CSF2, CXCL1, SERPINB7, MMP10 compared to organoids, MSCM, FM2 and ECM monocultured cells (FIG. 7J). The inductions of these genes in the other ECM-FM-2:ORG co-cultures was lower but still significantly compared to monocultures. These molecules play diverse roles in regulating immune responses, inflammation, tissue remodeling, and homeostasis. In contrast, the upregulation of mucin like gene MUCL3 was higher in co-cultures with an elevated ratio of ECM cultured cells or organoids co-cultures compared to organoids, FM2 and ECM cells monocultures.

### Example 6: Influence of Stromal Cells on Tumor Response to Chemotherapy: Insights from Co-Culture Models

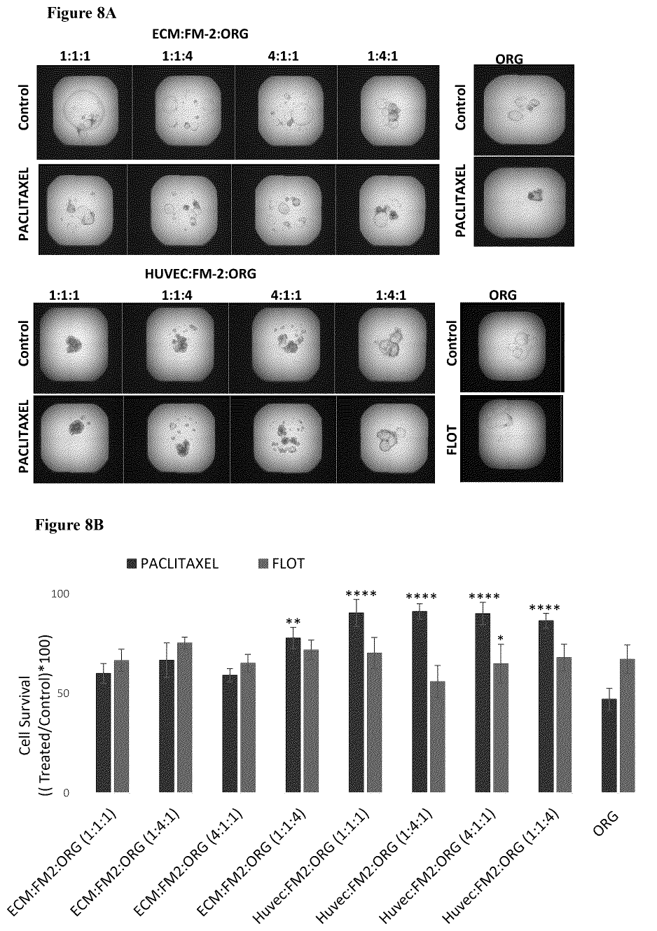

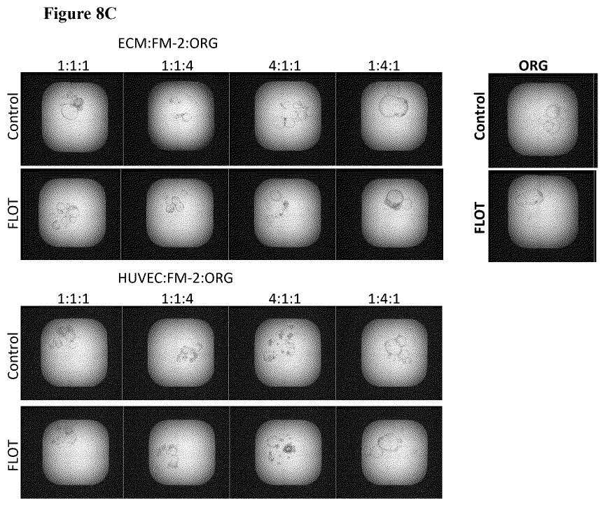

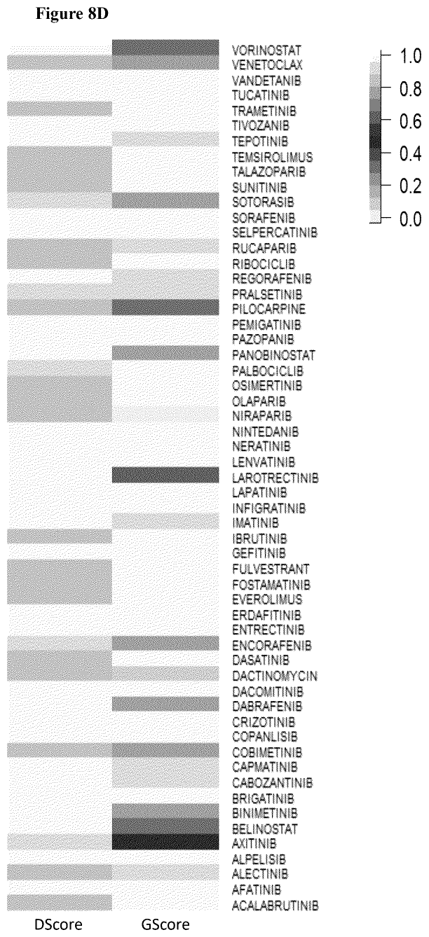

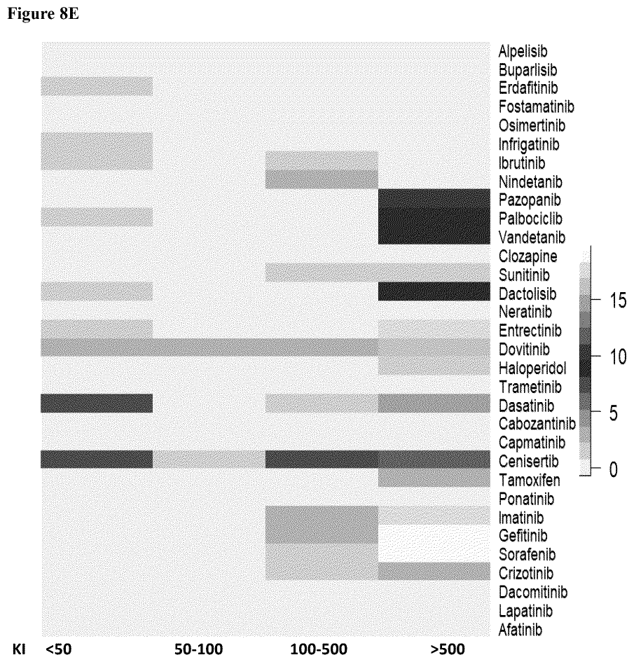

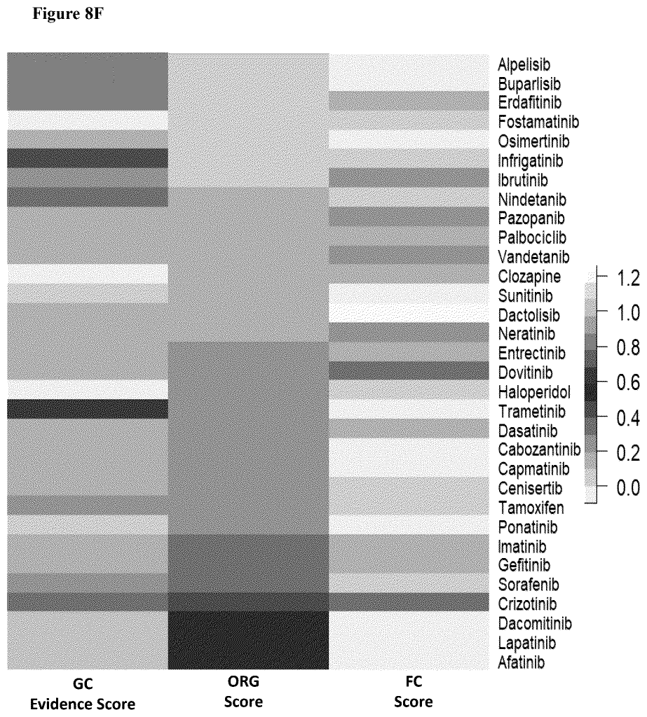

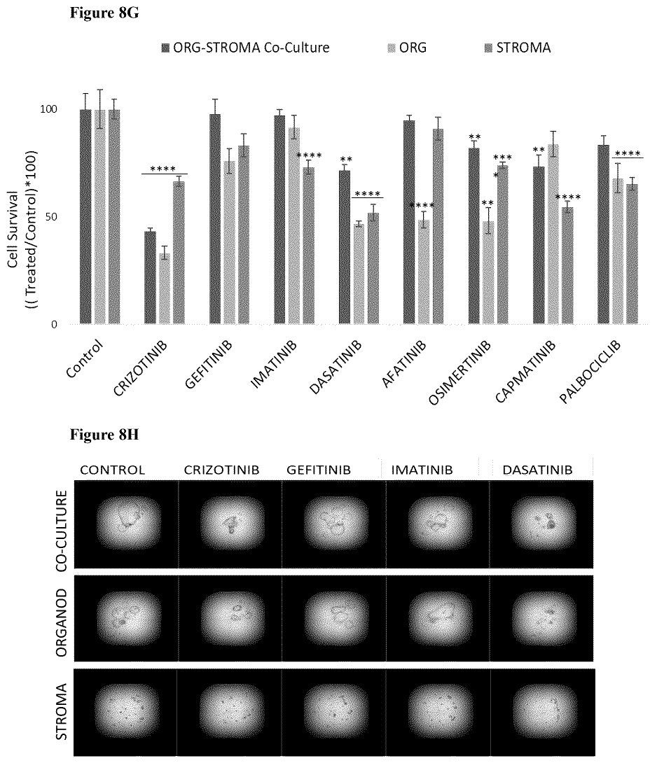

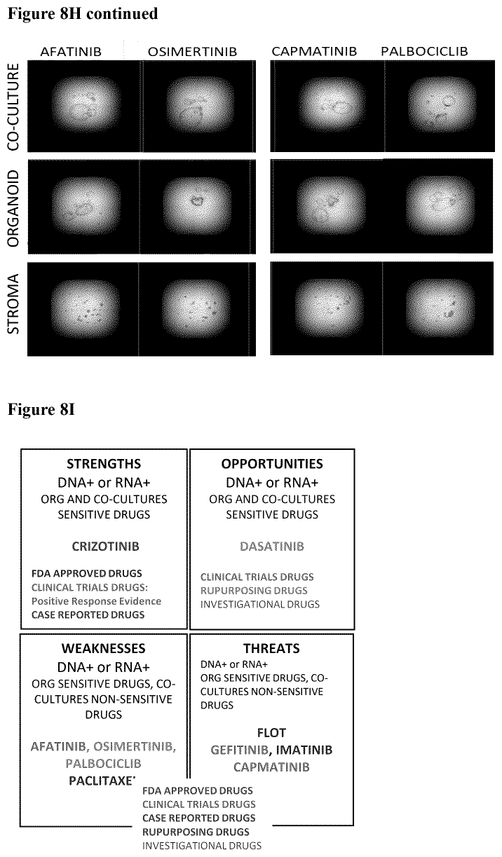

To assess the impact of stromal cells on tumor cell response to chemotherapy, drug cytotoxicity was compared in co-cultures and organoids derived from the same tumor sample across various ratios of organoids and stroma cells over a 3-day period. Treatment with FLOT resulted in a similar approximately 30% decrease in cell viability in both organoids and organoid-stroma cell co-cultures. However, paclitaxel treatment led to a notable 53% (p=0.0007) reduction in cell viability in organoids. While there were no significant differences in cell viability among organoids and TECs:TASCs:Org co-cultures at ratios of 1:1:1, 4:1:1, and 1:4:1, they displayed reduced sensitivity to paclitaxel-induced cytotoxicity, showing approximately 40% (P<0.05) cell death. Notably, TECs:TASCs:Org co-cultures at a ratio of 1:1:4 exhibited 22% cell death (p=0.08), significantly different from organoids alone (p=0.016). Furthermore, co-cultures containing Huvec cells displayed resistance to paclitaxel treatment, resulting in approximately 10% cell death (FIG. 8A-8B). Paclitaxel treatment also reduced the size and affected the integrity of affected organoids and co-cultures. The effect of FLOT is less evident in bright field images (FIG. 8C).

### Example 7: Influence of Stromal Cells on Tumor Response Targeted Therapy: Insights from Co-Culture Models and Transcriptomic Prioritization Sensitivity

Targeted drugs for a specific patient were prioritized based on transcriptomic data from the patient's tumor derived organoids and stroma cells. Out of the 1192 genes differentially upregulated in organoids compared to stroma cells (log 2FC>1, p<0.05, and n counts>=30), only 309 were found to be related to drugs using the PanDrugs platform database (FIG. 8D). The drugs were scored based on the number of genes that have a drug score higher than 0.75 and low constant inhibition or IC50 value (FIG. 8E). Then, drugs were further prioritized based on an RNA expression score, which considered the organoids' expression level of these drug-related genes, an RNA fold change score representing the fold change expression of these genes in the organoids relative to stroma cells and an RNA level of evidence score. This latter score ranked published evidence regarding the relation of these genes with GC, taking into consideration factors such as the existence of drugs targeting the specific gene (clinical use, clinical trial phase) and the role of the gene in GC oncogenesis, cell proliferation, cell death, and progression (FIG. 8F).

The cytotoxic activity of targeted drugs selected from this analysis, was then tested in organoids as compared to co-culture using concentrations found in peripheral blood. Upon comparison between organoids, stroma cells, and their co-culture, it was observed that after crizotinib treatment, both organoids and stroma cells-organoid co-cultures experienced more than 50% (p=0.0013 and p-0.0023 respectively) cell death. Afatinib and osimertinib treatments resulted in approximately 50% (p=0.0018 and p-0.016 respectively) organoid cell death. However, the presence of stroma cells in the co-culture rendered the organoids resistant to these treatments. While dasatinib decreased the viability of both organoids and stroma cells monocultures, its cytotoxic effect was diminished in the co-culture model. Notably, neither the organoids nor the co-culture showed significant response to gefitinib, imatinib, or capmatinib treatments. Interestingly, stroma cells monocultured showed decreased viability following capmatinib and imatinib treatments (FIG. 8G). The cytotoxicity of the drugs was also visualized in bright field images by observing a decrease in the size of co-cultures or organoids (FIG. 8H).

The analyzed drugs were categorized using a Strengths, Weaknesses, Opportunities, and Threats (SWOT) analysis to determine their potential effectiveness for a specific patient. Strengths included FDA-approved drugs with high RNA expression, fold change, and level of evidence scores, as well as high cytotoxic activity in the co-culture model, exemplified by crizotinib. Opportunities involved repurposing drugs and targeted drugs in clinical trials, with favorable scores in RNA expression, fold change, and level of evidence, and demonstrated cytotoxicity in both co-culture and organoid models, such as dasatinib. Weaknesses encompassed drugs causing up to 30% cell death in the co-culture but more than 50% cell death in organoids, including paclitaxel, FLOT, afatinib, and osimertinib. Threats consisted of drugs with low RNA scores or no cytotoxicity in the ex-vivo assay, such as gefitinib, imatinib, and capmatinib, identified in the analysis (FIG. 8I).

### Example 8: Human Kidney Cancer Tumor Avatar

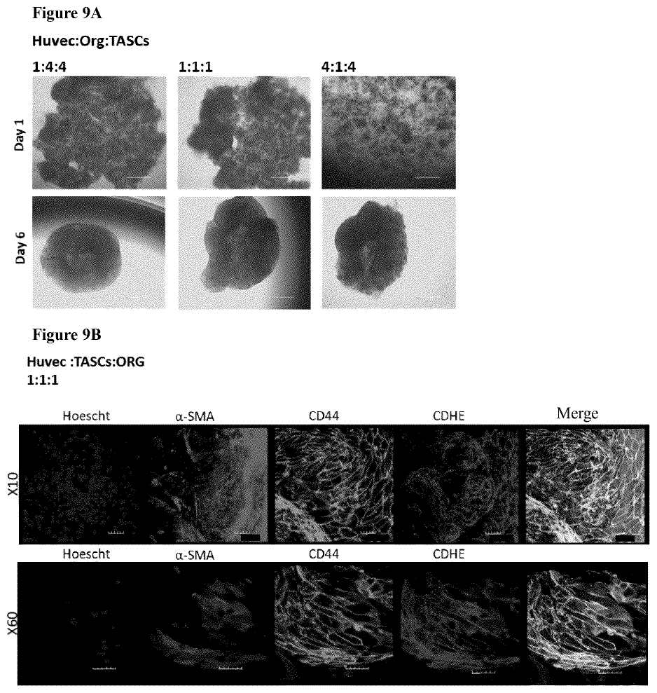

Next, the inventors aimed to examine a tumor avatar model, composed of kidney tumor epithelial cells, TASCs and TECs, cultured in the presence of a Matrigel and fibronectin scaffold. TECs, TASCs and kidney organoid-derived tumor epithelial cells clusters (5-15 cells/cluster) were seeded at different ratios (1:1:1, 1:4:1 or 4:1:1 respectively). As shown in FIGS. 9A-9B, different subpopulations of TME cells were observed, including cancer associated fibroblasts (CAFs, α-SMA cells), and tumor associated mesenchymal stem cells (TA-MSCs, CD44 cells), in addition to the tumor epithelial cells (CDHE cells).

### Example 9: Pancreas Cancer Tumor Model

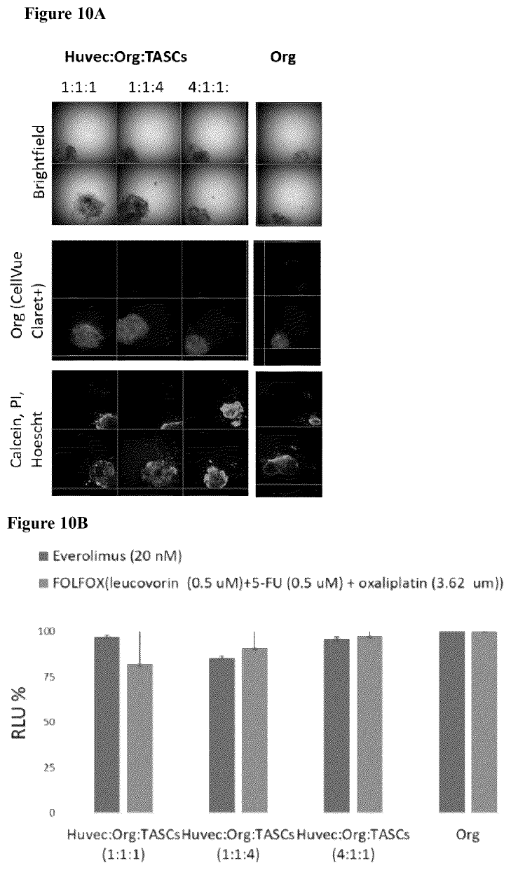

Next, the inventors aimed to examine a tumor avatar model, composed of pancreas tumor epithelial cells, TASCs and Huvec cells, cultured in the presence of a Matrigel and fibronectin scaffold. Huvec, TASCs and kidney organoid-derived tumor epithelial cells clusters (5-15 cells/cluster) were seeded at different ratios (1:1:1, 1:4:4 or 4:1:4 respectively) and cultured for 6 days. Tumor cells, labelled with CellVue Claret far red fluorochrome localized in the interior of the tumor avatar. In this patient, neither the organoids nor the tumor avatar models were affected by everolimus or FLOT treatment (FIG. 10A-10B).

### Example 10: Lung Cancer Tumor Model

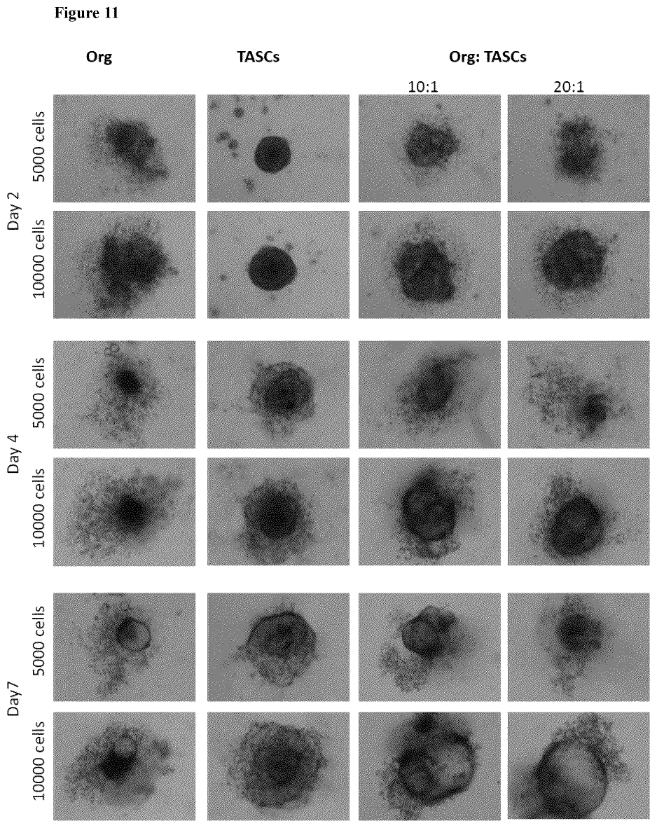

The inventors also examined a tumor avatar model, composed of lung tumor epithelial cells and TASCs, cultured in the presence of a Matrigel scaffold. The size of tumor avatars is positively correlated with the number of cells at the time of seeding. In this model, it could be observed that even the presence of a low percentage of TASCs in the tumor avatar had an effect on the spatial organization of the cells, as compared to organoids or TASCs cultured alone (FIG. 11).

In conclusion, it was surprisingly found that incorporation of patient-derived TME cells (e.g., TASCs and TECs) into a tumor avatar model, increases the sensitivity of cancer drug screening, and can be applied to a variety of cancer types, including gastric, kidney pancreas and lung cancers.

### Example 11: Tumor Avatar Production in a Matrigel Dome

The use of Matrigel domes has been shown to enhance organoid formation and survival. Further, it was hypothesized that this unique architecture could allow for immediate coculture of tumor cells and the various stromal cell types without the need for weeks of preculture in various media. The architecture of the dome already creates different growth regions that could allow for the specific expansion of specific types of cells, but all in one dish without the need for separate culture plates. It was hypothesized that the medium and scaffold composition can support tumor and stromal cells survival from day 0.

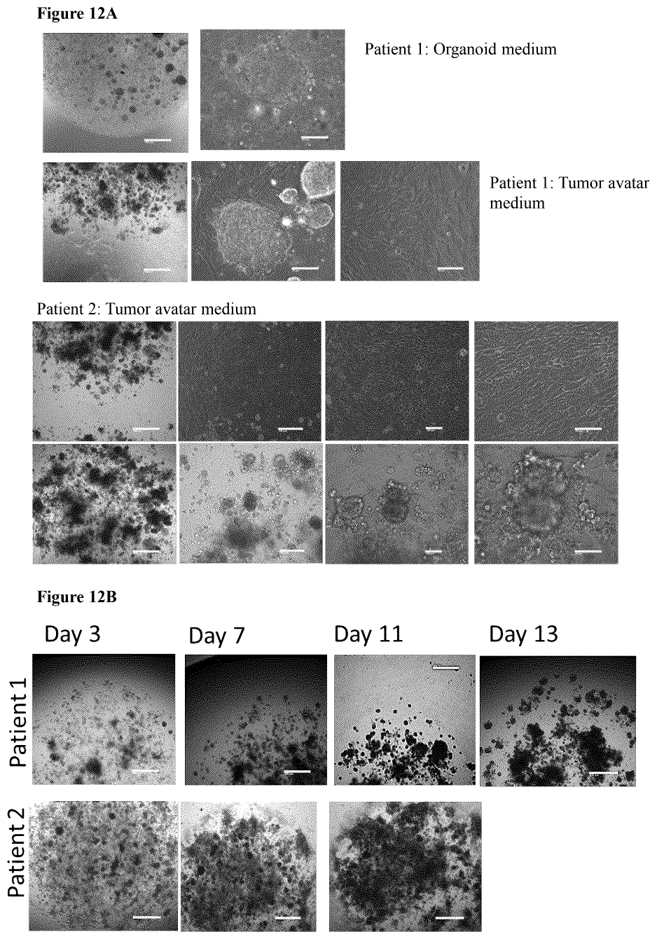

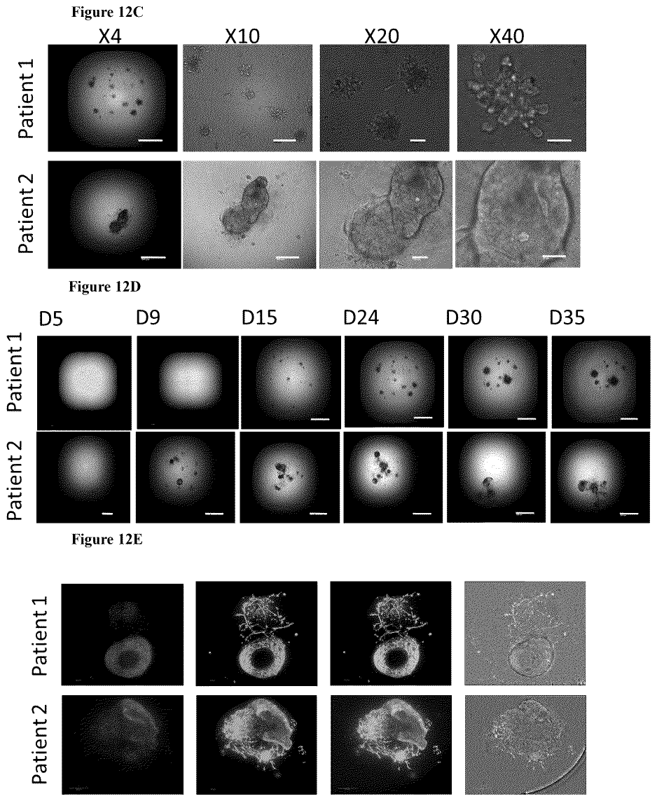

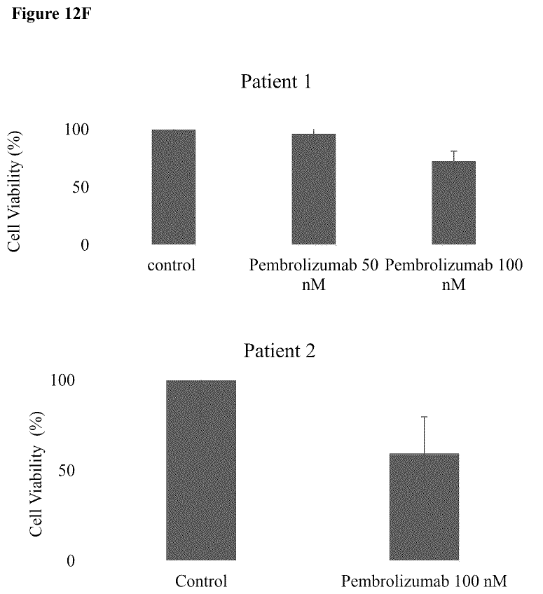

To test this gastric tumor avatars were generated from two different patient's cells, one with gastrointestinal sarcoma (patient 1) and one with gastric adenocarcinoma (patient 2). Cells were dissociated from gastric tissue and cultured in a Matrigel done in adherent plates with tumor avatar media (Table 2) or organoid media (FIG. 12A). After 7 days, organoids were formed in the dome while stromal cells and metastatic tumor cells invaded the rest of the tissue culture plate. Interestingly, cells from patient 1 could be cultured in organoid media; tumor cells from patient 2 did not survive in organoid media. However, when organoid media was used for patient 1, the number of stromal cells was greatly decreased and eventually these cells were lost in subsequent passages.

Importantly, it was noted that tumor avatar cells could be frozen and then defrosted and cultured again. These frozen cells showed similar morphology and cell type distributions to the tumor avatars that were not frozen (FIG. 12B).

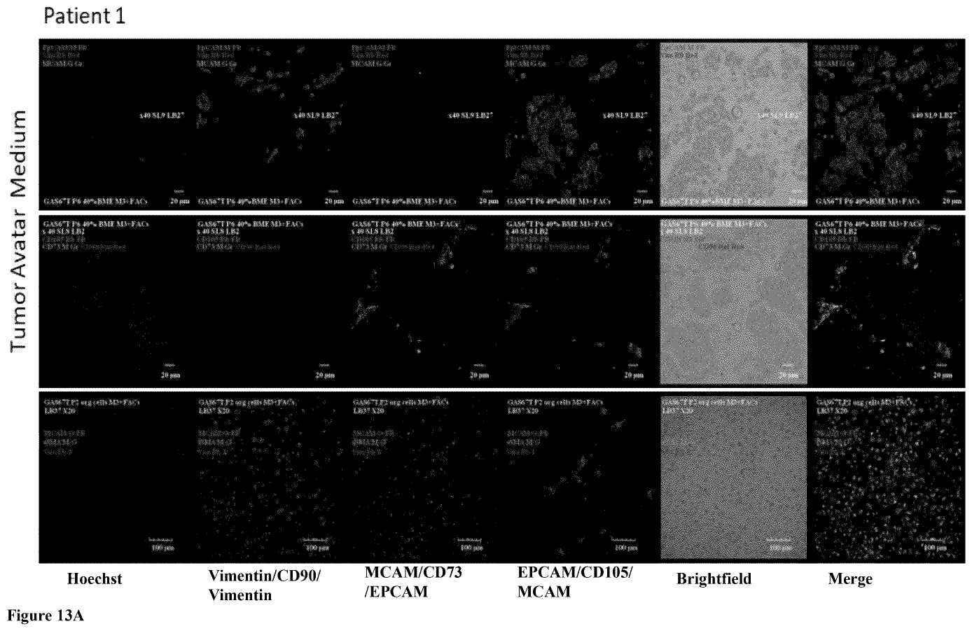

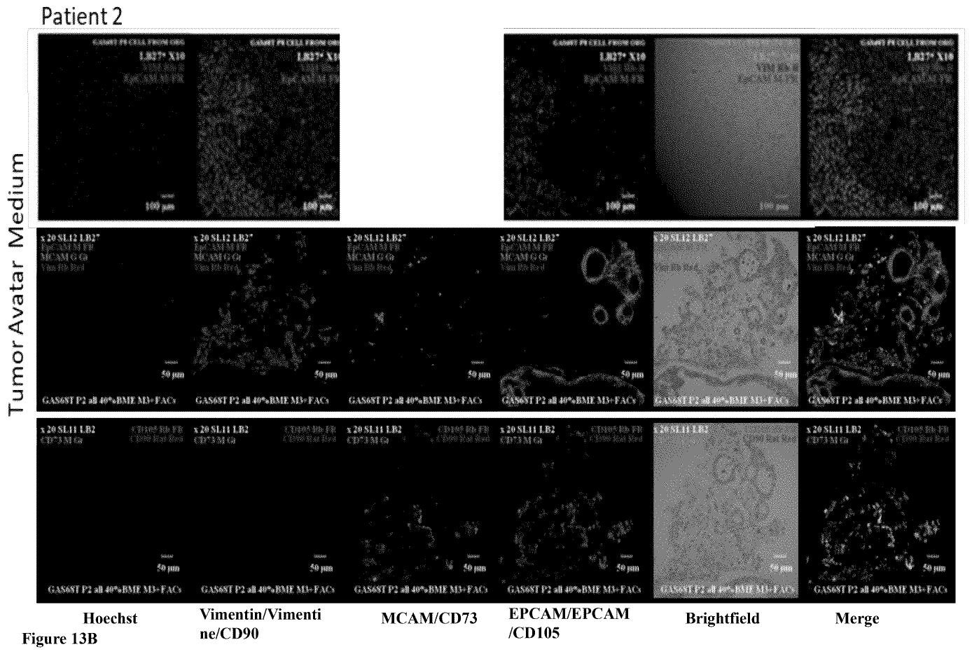

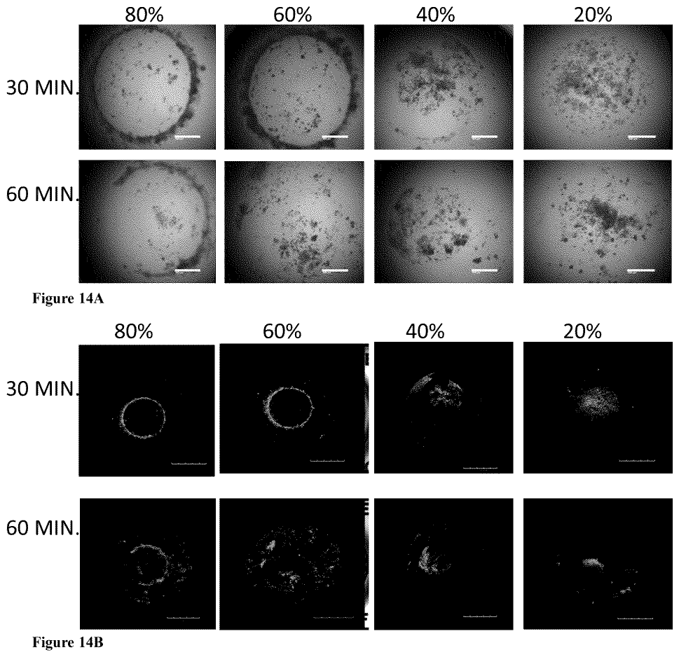

The cells from these patients were also cultured in the standard culture described above for producing a tumor avatar (preculture in various media, followed by mixed culture in low adherence plates). Tumor avatars were produced as before and then cultured in tumor avatar media (Table 3) with the expected morphology and cell type distribution produced (FIG. 12C-12E). It was observed that cells grown with the dome and avatar medium, organized in a similar manner and with the same cell types to that observed for the tumor avatar in grown by the standard method (FIG. 13A-13B). Drug screening experiments were also performed with avatars produced the standard way and with the dome and similar drug response results were observed (data not shown).

Lymphocytes were isolated from the peripheral blood of patient 1 and patient 2. CD45 staining was used to identify lymphocytes. The lymphocytes were added to the tumor avatar culture either from the beginning or on the day of drug testing. The presence of lymphocytes allowed for the testing of immunotherapy drugs. Pembrolizumab was added at various concentrations to the tumor avatars and cell viability was monitored for 72 hours after treatment using the Cell-Titer Glo assay. Pembrolizumab was found to be effective for treating both organoids and thus both patients. This result is confirmed by administering pembrolizumab to the patients who both respond.

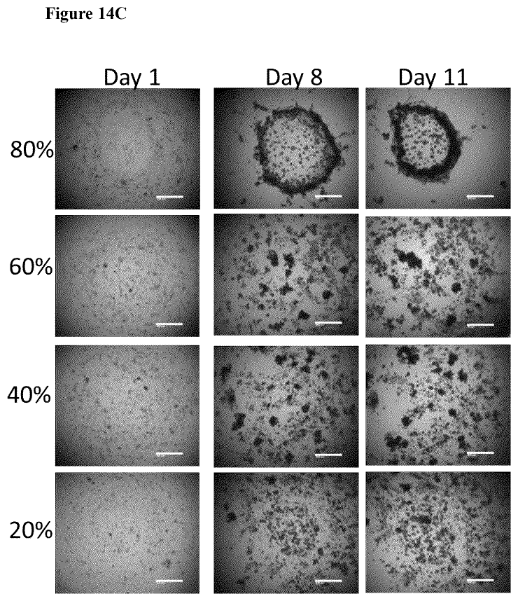

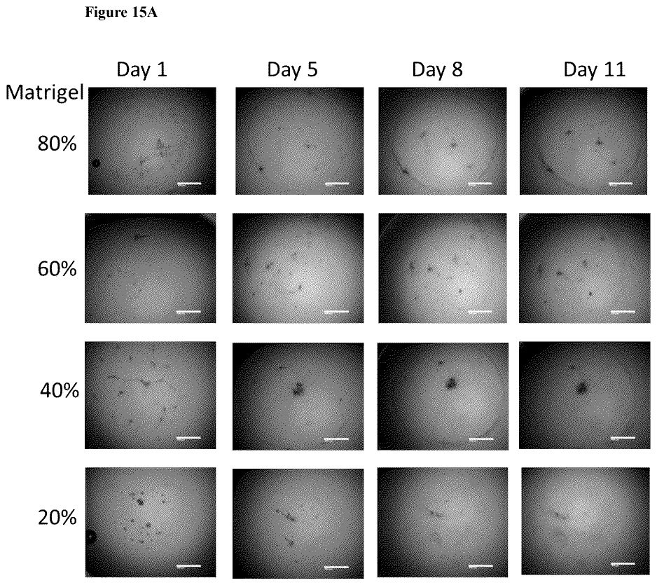

In order to test if the Matrigel dome would allow for distinct culturing of tumor cells and stromal cells in the same dish gastric cancer organoids were cultured on domes prepared with different concentrations of Matrigel (80%-20%). Domes were formed and cells were seeded onto the dome, incubated for 30 or 60 minutes in tumor avatar medium. Domes with Matrigel concentration of 40% or lower allowed organoids to grow on the domes, while at higher concentrations organoids remained outside the dome (FIG. 14A). Organoids were stained with CellTrace CFSE dye for easier visualization (FIG. 14B). Next, organoids were suspended in media with Matrigel at various concentrations and the domes were produced. This method of production allowed the organoids to form in and on the dome at all concentrations, though at 80% Matrigel many of the cells were forced out of the dome (FIG. 14C).

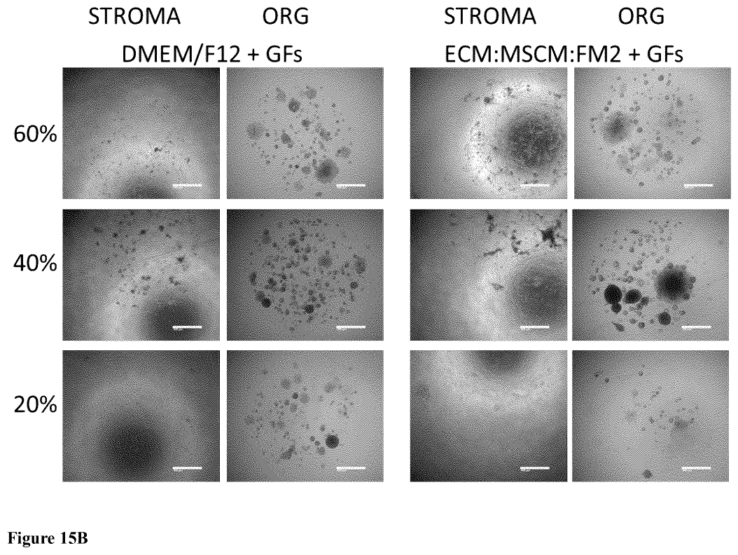

The same experiments were performed but with gastric tumor associated stromal cells. Stromal cells were seeded onto a previously formed dome and cultured for 11 days in mixed stromal cell media (ECM:MSCM:FM 1:1:1). Matrigel concentrations higher than 20% repelled stromal cells growth inside or on the domes (FIG. 15A). It was thus determined that a dome concentration of greater than 20% Matrigel but less than 60% would allow for separate culture of the tumor cells and stromal cells, as the organoids would form in the dome and the stromal cells would remain arounds the dome.

Next, gastric tumor stromal cells and organoids were resuspended in different concentrations of Matrigel and plated to form the dome. It was observed that whereas organoids remained in the dome, stromal cells moved out of the dome, toward the bottom of the well, at higher concentrations of Matrigel (FIG. 15B). At Matrigel concentrations of 20% stromal cells were observed in the dome and the rest of the well. Thus, in this set up as well Matrigel dome concentrations of around 40% (from 20-60) were ideal for compartmentalizing tumor growth to the dome and stromal cell growth in the surrounding dish.

TABLE 2
Tumor Avatar media for culturing starting at day 0

| Media component | | |
|---|---|---|
| DMEM/F12 or | | |
| ECM:FM2:MSCM(1:1:1) or | | |
| DMEM/F12:ECM:MSCM:ECM | Final concentration | Units |
| (1-10:1:1:1) | x1 | ml |
| R-spondin 1 | 500.00 | ng · ml⁻¹ |
| Noggin | 100.00 | ng · ml⁻¹ |
| Wn3ta | 100.00 | ng · ml⁻¹ |
| Gastrin | 100.00 | ng · ml⁻¹ |
| EGF | 50.00 | ng · ml⁻¹ |
| FGF10 | 10.00 | ng · ml⁻¹ |
| FGF-2 | 10.00 | ng · ml⁻¹ |
| Prostaglandin E2 | 1.00 | μM |
| Y-27632 | 5.00 | μM |
| A 83-01 | 500.00 | nM |
| Nicotinamide | 4.00 | mM |
| SB202190 | 0.50 | μM |
| N-Acetyl-L-cysteine | 1.00 | mM |
| Caspofungin | 0.00 | mg · ml⁻¹ |
| B27 | 1.00 | x |
| MEM -NEAA | 1.00 | ml |
| Sodium Piruvate | 1.00 | ml |
| VEGF | 5.00 | ng · ml⁻¹ |
| Cd Lipid Concentrate (optional) | x1 | ml |
| IL-6 | 20.00 | ng · ml⁻¹ |
| IL-8 | 10.00 | ng · ml⁻¹ |
| Ascorbic Acid | 50.00 | ug · ml⁻¹ |
| Heparin | 22.50 | ug · ml⁻¹ |
| PDGF (optional) | 25.00 | ng · ml⁻¹ |
| BMP4 | 50.00 | ng · ml⁻¹ |
| BMP7 | 50.00 | ng · ml⁻¹ |
| Trombospondin (optional) | 5-50 | ug · ml⁻¹ |
| Hydrocortisone (optional) | 0.20 | ug · ml⁻¹ |
| Hepes | 10.00 | mM |
| ABAM | x1 | ml |
| GlutaMax Supplement | x1 | ml |
| Matrigel | 5.00 | % |
| Fibronectin | 5-10 | ng · μl⁻¹ |
| FBS (optional) | 5.00 | % |
| IL-2 (optional) | 50.00 | Units/ml |
| β-mercaptoethanol (optional) | 50.00 | μM |
| M-CSF (optional) | 50.00 | ng · ml⁻¹ |

TABLE 3
Tumor Avatar media for after preculturing of stromal cells

| Media component | | |
|---|---|---|
| DMEM/F12 or | | |
| ECM:FM2:MSCM(1:1:1) or | | |
| DMEM/F12:ECM:MSCM:ECM | Final concentration | Units |
| (1-10:1:1:1) | x1 | ml |
| R-spondin 1 | 500.00 | ng · ml − 1 |
| Noggin | 100.00 | ng · ml − 1 |
| Wn3ta | 100.00 | ng · ml − 1 |
| Gastrin | 100.00 | ng · ml − 1 |
| EGF | 50.00 | ng · ml − 1 |
| FGF10 | 10.00 | ng · ml − 1 |
| FGF-2 | 10.00 | ng · ml − 1 |
| Prostaglandin E2 | 1.00 | μM |
| A 83-01 | 500.00 | nM |
| Nicotinamide | 4.00 | mM |
| N-Acetyl-L-cysteine | 1.00 | mM |
| Caspofungin | 0.00 | mg · ml − 1 |
| B27 | 1.00 | x |
| MEM -NEAA | 1.00 | ml |
| Sodium Piruvate | 1.00 | ml |
| VEGF | 5.00 | ng · ml − 1 |
| Cd Lipid Concentrate (optiona) | x1 | ml |
| IL-6 | 20.00 | ng · ml − 1 |
| IL-8 | 10.00 | ng · ml − 1 |
| Ascorbic Acid | 50.00 | ug · ml − 1 |
| Heparin | 22.50 | ug · ml − 1 |
| PDGF (optional) | 25.00 | ng · ml − 1 |
| BMP4 | 50.00 | ng · ml − 1 |
| BMP7 | 50.00 | ng · ml − 1 |
| Trombospondin (optional) | 5-50 | ug · ml − 1 |
| Hydrocortisone (optional) | 0.20 | ng · ml − 1 |
| Hepes | 10.00 | mM |
| ABAM | 1.00 | ml |
| GlutaMax Supplement | 1.00 | ml |
| Matrigel | 5.00 | % |
| Fibronectin | 5-10 | ng · μl − 1 |
| FBS (optional) | 5.00 | % |
| IL-2 (optional for immune cells) | 50.00 | Units · ml − 1 |
| β-mercaptoethanol | 50.00 | μM |
| (optional for immune cells) | | |
| M-CSF (optional for immune cells) | 50.00 | ng · ml − 1 |
| IL-4 (optional for immune cells) | 20.00 | ng · ml − 1 |
| IL-13 (optional for immune cells) | 20.00 | ng · ml − 1 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1-53. (canceled)

54. A three-dimensional (3D) tumor avatar comprising: an artificial scaffold, tumor cells, and tumor microenvironmental (TME) cells comprising tumor associated endothelial cells (TECs) and cancer associated fibroblasts (CAFs).

55. The 3D tumor avatar of claim 54, wherein any one of: (i) said TME cells are derived from a tumor tissue or a surrounding tissue thereof; (ii) said TME cells and said tumor cells belong to a single histologic type of said tumor, optionally wherein said histologic type is carcinoma or sarcoma; and (iii) said TME cells and said tumor cells belong to a single primary or metastatic site of said tumor.

56. The 3D tumor avatar of claim 55 wherein said primary site of said tumor is selected from the group consisting of: gastrointestinal cancer, kidney cancer, liver cancer, breast cancer, bladder cancer, prostate cancer, ovary cancer, uterine cancer, head and neck cancer, thyroid cancer, glioma, neuroblastoma, melanoma, and lung cancer.

57. The 3D tumor avatar of claim 54, wherein said 3D tumor avatar is a patient-personalized tumor avatar, and wherein said TECs and said CAFs are present in said patient-personalized tumor avatar at a ratio found in a sample obtained or derived from said patient.

58. The 3D tumor avatar of any one of claim 54, wherein the ratio between tumor cells and CAFs in said 3D tumor avatar is between 1:10 and 1:1, the ratio between tumor cells and TECs in said 3D tumor avatar is between 1:10 and 1:1, or both.

59. The 3D tumor avatar of any claim 54, wherein said artificial scaffold comprises Matrigel, vitronectin, fibronectin, or any combination thereof.

60. The 3D tumor avatar of claim 56, wherein said artificial scaffold is selected from: (i) an artificial scaffold comprising a Matrigel dome comprising a Matrigel concentration of at least 20%; and (ii) an artificial scaffold comprising a concentration gradient of Matrigel, fibronectin, vitronectin or any combination thereof, and wherein said gradient of Matrigel is from 1-100% concentration, said gradient of fibronectin is from 0-50 ng/ul and said gradient of vitronectin is from 0-50 ng/ul.

61. The 3D tumor avatar of any one of claim 54, wherein said CAFs comprise inflammatory CAFs (iCAFs) and extracellular matrix-remodeling CAFs (eCAFs).

62. The 3D tumor avatar of any one of claim 54, further comprising any one of: (i) tumor associated mesenchymal cells (TAMCs), optionally wherein said TAMCs are tumor associated mesenchymal stem cells (TA-MSCs); (ii) peripheral blood lymphocytes (PBLs); and (iii) tumor associated adipocytes (TAAs), tumor associated immune cells, or both.

63. A kit comprising a 3D tumor avatar of claim 54 and a liquid media suitable for in vitro organization and maintenance of said 3D tumor avatar.

64. The kit of claim 63, wherein said media comprises at least one component selected from the group consisting of: hepes, abam, glutamax, non-essential amino acids, sodium pyruvate, R-Spondin, noggin, insulin growth factor 1 (IGF1), fibroblast growth factor 2 (FGF2), FGF10, epidermal growth factor (EGF), vascular endothelial ascorbic acid, growth factor (VEGF), Platelet-derived growth factor (PDGF), interleukin 6 (IL-6), IL-8, BMP4, BMP7, heparin, hydrocortisone, trombospondin, indomethacine, 3-Isobutyl-1-methylxanthine (IBMX), intralipids, bone morphogenetic protein 7 (BMP7), B27, A 83-01, N-Acetyl-L-cysteine, nicotinamide, and Y-27632.

65. The kit of any one of claim 63, wherein said liquid media comprises DMEM/F12, DMEM/F12:ECM:MSCM:ECM (1-10:1:1:1) or ECM:MSCM:ECM (1:1:1) media.

66. The kit of claim 63, wherein at least one of:
a. said tumor cells are derived from a gastrointestinal cancer, and said media further comprises at least one component selected from the group consisting of: Wnt3a, gastrin, HGF and prostaglandin E2;
b. said tumor cells are derived from a kidney cancer, and said media further comprises at least one component selected from the group consisting of: HGF, epinephrine, hydrocortisone and FGF8;
c. said tumor cells are derived from a lung cancer, and said media further comprises FGF7;
d. said tumor cells are derived from a bladder cancer, and said media further comprises FGF7, heregulin or both; and
e. said tumor cells are derived from a breast, ovary or uterine cancer, and said media further comprises at least one component selected from the group consisting of: B-Estradiol, hydrocortisone, and heregulin.

67. A method for producing a 3D tumor avatar, the method comprising:
a) providing tumor cells and tumor microenvironmental (TME) cells comprising tumor associated endothelial cells (TECs) and cancer associated fibroblasts (CAFs);
a) culturing said tumor cells and said TME cells with an artificial scaffold in a liquid media suitable for in vitro organization and maintenance of said 3D tumor avatar;
thereby producing a 3D tumor avatar.

68. The method of claim 67, further comprising before step (a): isolating said tumor cells, said TME cells, or both, from a tumor tissue or a surrounding tissue thereof.

69. The method of claim 67, wherein said providing TME cells comprises receiving isolated stromal cells from a tumor or a surrounding tissue thereof and culturing a first portion of said received isolated stromal cells in endothelial cell media (ECM) and culturing a second portion of said received isolated stromal cells in fibroblast medium (FM).

70. The method of claim 67, wherein any one of: (i) said culturing in ECM is for a sufficient time to enrich for iCAFs and wherein said culturing in FM is for a sufficient time to enrich for eCAFs, optionally wherein said culturing said first portion and culturing said second portion is for 1-3 weeks; (ii) said providing TME cells comprises culturing a third portion of said received isolated stromal cells in mesenchymal stem cell medium (MSCM) and/or culturing a fourth portion of said received isolated stromal cells in MSCM:ECM:FM (1:1:1); and both (i) and (ii).

71. The method of 67, wherein said culturing of step (b) is in liquid media comprising
a. DMEM/F12, ECM:FM-2:MSCM (1:1:1) or DMEM/F12:ECM:MSCM:FM-2 (1-10:1:1:1); and
b. Hepes, ABAM, 2 GlutaMax supplement, R-spondin 1, Noggin, Wnt3a, epidermal growth factor (EGF), Gastrin, fibroblast growth factor 2 (FGF2), FGF10, gastrin, prostaglandin E2, A83-01, nicotinamide, and Vascular endothelial growth factor (VEGF), interleukin-6 (IL-6), IL-8, Bone Morphogenetic Protein 4 (BMP4) and BMP7 to produce said organoids, and optionally Y-27632, SB202190, trombospondin and hydrocortisone.

72. A 3D tumor avatar produced by the method of claim 67.

73. A method for screening a drug for being suitable as an anticancer drug, the method comprising:
a) culturing a 3D tumor avatar of claim 54 in a culture media,
b) contacting said 3D tumor avatar with said drug; and,
c) measuring an anticancer effect in said 3D tumor avatar, wherein presence of said effect above a predetermined threshold indicates said drug is suitable for being an anticancer drug;
thereby screening a drug or combination of drugs for being suitable as an anticancer drug.
